(19) United States
(12) Patent Application Publication
(10) Pub. No.: US 2002/0068342 A1
(43) Pub. Date: Jun. 6, 2002

(54) Novel nucleic acid and amino acid sequences and novel variants of alternative splicing

(76) Inventors: Rami Khosravi, Herzliya (IL); Jeanne Bernstein, Kfar Yona (IL)

Correspondence Address: BIRCH STEWART KOLASCH & BIRCH, PO BOX 747, FALLS CHURCH, VA, 22040-0747, US

(21) Appl. No.: 09/778,927

(22) Filed: Feb. 8, 2001

(30) Foreign Application Priority Data
Feb. 9, 2000 (IL) 2000/134453
Mar. 29, 2000 (IL) 2000/135341

(51) Int. Cl. C12N 9/00; C07H 21/04; C12P 21/02; C12N 5/06

(52) U.S. Cl. 435/183; 435/325; 435/320.1; 435/69.1; 536/23.2

(57) ABSTRACT

The present invention concerns nucleic acid sequences and amino acid sequences. The sequences include sequences coding for variants obtained by alternative splicing, for homologs of the known thrombopoietins and sequences that encode for novel homologs of transporter proteins

### FIELD OF THE INVENTION

The present invention concerns novel nucleic acid sequences, vectors and host cells containing them, amino acid sequences encoded by said sequences, and antibodies reactive with said amino acid sequences, as well as pharmaceutical compositions comprising any of the above. The present invention further concerns methods for screening for candidate activators or deactivators utilizing said amino acid sequences.

### BACKGROUND OF THE INVENTION

Alternative splicing (AS) is an important regulatory mechanism in higher eukaryotes (P. A. Sharp, Cell 77, 805-8152 (1994). It is thought to be one of the most important mechanisms for differential expression related to tissue or development stage specificity. It is known to play a major role in numerous biological systems, including human antibody responses, and sex determination in Drosophila, (S. Stamm, M. Q. Zhang, T. G. Marr and D. M. Helfinan, Nucleic Acids Research 22, 1515-1526 (1994); B. Chabot, Trends Genet. 12, 472-478 (1996); R. E. Breitbart, A. Andreadis, B. Nadal-Ginard, Annual Rev. Biochem., 56, 467-495 (1987); C. W. Smith, J. G. Patton, B. Nadal-Ginard, Annu. Rev. Genet., 27, 527-577 (1989)).

Until recently it was commonly believed that alternative splicing existed in only a small fraction of genes (about 5%). A recent observation based on literature survey of known genes revises this conservative estimate to as high as an estimate that at least 30% of human genes are alternatively spliced (M. S. Gelfand, I. Dubchak, I. Draluk and M. Zorn, Nucleic Acids Research 27, 301-302 (1999). The importance of the actual frequency of this phenomenon lies not only in the direct impact on the number of proteins created (100,000 human genes, for example, would be translated to a much higher number of proteins), but also in the diversity of functionality derived from the process.

Several mechanisms at different stages may be held responsible for the complexity of higher eukalyote which include: alternative splicing at the transcription level, RNA editing at the post-transcriptional level, and post-translational modifications are the ones characterized to date.

Thrombopoietin (TPO) is a central regulator of the megakaryocytopoiesis. This molecule is lineage-specific cytokine affecting the proliferation and maturation of megakaiyocytes from their committed progenitor cells and acts at a late stage of megakaryocyte development. It may be the major physiological regulator of circulating platelets. Two forms of TPO have been entered at clinical trials, the full length recombinant version (THTOP) and a truncated form. The administration of both forms cause a dose-dependent increase in platelet count with no affect on white cell count or hematacrit. The platelets induced by these two forms are morphologically and functionally normal. When administered following myello suppressive chemotherapy the two forms of TPO significantly enhance platelet recovery. TPO progenator cells of multiple hematopoietic lineage may enhance the effect of filgrastim on peripheral blood progenator cell levels after chemotherapy.

The regulated exocytotic release of neurotransmitters in response to neural activity requires storage within intracellular vesicles. In the nervous system, these vesicles are the synaptic vesicles that are derived from the endosomal compartment, whereas in endocrine cells larger secretory granules, such as the chromaffin granules of adrenal medulla, are derived from the trans golgi networks. For classical transmitters, that are synthesized in the cytoplasm or appear there after removal from the synapses by plasma membrane reuptake, storage depends upon the active transport into the vesicles. Several distinct transport activities have been identified for monoamines, acetylcholine, glutamate, GABA and glycine. Vesicular monoamine transporters (VMATs) catalyze transport and storage of monoamines, serotonin, dopamine, norepinephrine, epinephrine, and histamine. The driving force utilized by the VMAT is the H+ electrochemical gradient generated by the vacuolar ATP-dependent H+ pump (V-ATPase) located on vesicular plasma membrane. VMAT is inhibited by a wide variety of compounds including reserpine and tetrabenazine. In contrast to the plasma membrane transporters for dopamine, norepinephrine, and 5-HT which show relative substrate specificity, the monoamine transporter recognizes various monoamine with similar affinity.

All characterized monoaminegenic cells utilize the monoamine transporters for the vesicular accumulation of monoaminees prior to their release. This system operates in neuronal (catecholaminergic, serotoninergic or histaminergic) as well as in endocrine or neuroendocrine cells including endocrine cells of the gastric epithelium which secrete biologically active peptides and small messenger molecules such as histamine, serotonin, and gamma-aminobutyric acid.

The monoaminees, serotonin, dopamine, norepinephrine, epinephrine and histamine, play a crucial role in the function of the hypothalamic-pituitary-adrenal axis and in the integration of information in sensory, limbic, and motor systems. The monoaminee transporters are the most important determinants of extracellular levels of monoaminees, which act by packaging monoaminees into synaptic and secretory vesicles by exchange of protons.

An essential property of synaptic transmission is the rapid termination of action following neurotransmitter release. For many neurotransmitters, including catecholamine, serotonin, and certain amino acids (e.g., gamma-aminobutylic acid (GABA), glutamate and glycine), rapid termination of synaptic action is achieved by the uptake of the neurotransmitter into the presynaptic terminal and surrounding glial cells. This rapid re-accumulation of a neurotransmitter is the result of re-uptake by the presynaptic terminals. At presynaptic terminals, the various molecular structures for re-uptake are highly specific for such neurotransmitters as choline and the biogenic amines (low molecular weight neurotransmitter substances such as dopamine, norepinephrine, epinephrine, serotonin and histamine). These molecular structures are termed “transporters”. The transporters move neurotransmitter substances from the synaptic cleft back across the cell membrane of the presynaptic neuron and into the cytoplasm of the presynaptic terminus thus terminating the function of these substances. Inhibition or stimulation of neurotransmitter uptake provides a means for modulating the effects of the endogenous neurotransmitters.

Transporters of sugars are responsible for change in sugar concentrations according to membranes. For example the glucose transporter enables half of the glucose present inside the cell to leave within four seconds at normal body temperature. The glucose transporter operates through conformational change that the transporter undergoes while moving glucose across the membranes. Alternating between two conformations it moves its glucose-binding site from one side of the membrane to another. By “flipping” between its two conformational states the transporter facilitates the diffusion of glucose, i.e. it enables the glucose to avoid the barrier of the plasmid membrane while moving spontaneously down its concentration gradient so that when concentration reaches equilibrium net movement of glucose ceases.

The beta-galactosides are transported into cells by the lactose-protein symport which utilized the potential energy present in the protein gradiant transmitting simultaneously also a protein. Other transporters are known such as the multidrug Efflux Transporter 2.

### GLOSSARY

In the following description and claims use will be made, at times, with a variety of terms, and the meaning of such terms as they should be construed in accordance with the invention is as follows:

“Variant nucleic acid sequence”—the sequence shown in any one of SEQ ID NO: 1 to SEQ ID NO: 28, sequences having at least 90% identity (see below) to said sequence and fragments (see below) of the above sequences of least 20 b.p. long. These sequences are sequences coding for a novel, naturally occurring, alternative splice variants of native and known genes. It should be emphasized that the novel variants of the present invention are naturally occurring sequences resulting from alternative splicing of genes and not merely truncated, mutated or fragmented forms of known sequences which are artificially produced.

“Thrombopoietin-like sequence (TL)”—the sequence shown in any one of SEQ ID NO: 29 or 30, sequences having at least 70% identity to said sequences and fragments of the above sequence being 20 b.p. long. The two sequences are sequences coding for homologs of the known thrombopoietins and are both splice variants. It should be noted that the term “TL” does not necessarily signify that the TL protein coded by the above sequences has the same, or even similar physiological effect as the known Thrombopoietin, merely that it shows sequence homology with the known thrombopoietin.

“Transporter protein homolog (TH)”—the sequence shown in any one of SEQ ID NOs: 31 to 41, sequences having at least 70% identity to said sequence, and fragments of the above sequences being 20 b.p. long. These sequences have homology to various vesicular neurotransmitter transporters, such as to the monoamine transporters and features homology to sugar and other transporters of C. Elegance. SEQ ID NO: 38 and SEQ ID NO: 39 are in fact splice variants of one another, and SEQ ID NO: 40 and SEQ ID NO: 41 are updates of SEQ ID Nos 38 and 39.

The term TH does not necessarily signify that the protein coded by the above sequences has the same or even similar physiological activities to known transporters, especially of monoamines or amines or sugars and merely indicates that it shows sequence homology with these transporters.

“Variant product—also referred at times as the “variant protein” or “variant polypeptide”—is an amino acid sequence encoded by the variant nucleic acid sequence of SEQ ID NOS: 1 to 28 which is a naturally occurring mRNA sequence obtained as a result of alternative splicing. The amino acid sequence may be a peptide, a protein, as well as peptides or proteins having chemically modified amino acids (see below) such as a glycopeptide or glycoprotein. One example of products are shown in any one of SEQ ID NO: 42 to SEQ ID NO: 69. The term also includes homologues (see below) of said sequences in which one or more amino acids has been added, deleted, substituted (see below) or chemically modified (see below) as well as fragments (see below) of this sequence having at least 10 amino acids.

“TL product”—is an amino acid sequence coded by any one of SEQ ID NO: 29 or 30. An example of this sequence is a sequence of SEQ ID NO: 70 which is a common sequence coded by both SEQ ID NO: 29 and SEQ ID NO: 30. The amino acid sequence may be a peptide, a protein, as well as peptides or proteins having “chemically modified” (see below) amino acids such as glycopeptide or glycoprotein.

“TH product”—is am amino acid sequence coded by SEQ ID NO: 31 to SEQ ID NO: 41. The amino acid sequence may be a peptide, a protein, as well as peptides or proteins having chemically modified amino acids (see below) such as glycopeptides or glycoproteins. Examples of such MTPH-products are shown in SEQ ID NO: 71 to SEQ ID NO: 81. This term also includes analogs of said sequence in which one or more amino acids has been added, deleted, substituted (see below) or chemically modified (see below) as well as fragments of this sequence having at least ten amino acids.

“Nucleic acid sequence”—a sequence composed of DNA nucleotides, RNA nucleotides or a combination of both types and may includes natural nucleotides, chemically modified nucleotides and synthetic nucleotides.

“Amino acid sequence”—a sequence composed of any one of the 20 naturally appearing amino acids, amino acids which have been chemically modified (see below), or composed of synthetic amino acids.

“Fragment of variant nucleic acid sequence”—novel short stretch of nucleic acid sequences of at least 20 b.p., which does not appear as a continuous stretch in the original nucleic acid sequence of the variant (see below). The fragment may be a sequence which was previously undescribed in the context of the published RNA and which affects the amino acid sequence encoded by the known gene. For example, where the variant nucleic includes a sequence which was not included in the original sequence (for example, a sequence which was an intron in the original sequence) the fragment may contain said additional sequence. The fragment may also be a region which is not an intron, which was not present in the original sequence. For example where the variant lacks a non-terminal region, which was present in the original sequence. The two stretches of nucleotides spanning this region (upstream and downstream) are brought together by splicing in the variant, but are spaced from each by the spliced out region in the original sequence and are thus not continuous in the original sequence. A continuous stretch of nucleic acids comprising said two sparing stretches of nucleotides is not present in the original sequence and thus falls under the definition of fragment.

“Fragment of TL sequence/Fragment of TH sequence”—a continuous portion, preferably of about 20 nucleic acid sequences of the TL or TH sequences, respectively.

“Fragments of variant products”—novel amino acid sequences coded by the “fragment of valiant nucleic acid sequence” defined above.

“Fragments of TL/TH product”—a polypeptide which has an amino acid sequence which is the same as pair of but not all of the amino acid sequences of the TL/TH products, respectively.

“Homologues of variants”—amino acid sequences of variants in which one or more amino acids has been added, deleted or replaced. The addition, deletion or replacement should be in the regions or adjacent to regions where the variant differs from the original sequence (see below).

“Homologues of TL/TH”—an amino acid sequence of the TL/TH, respectively, in which one or more amino acids has been added, deleted or replaced.

“Conservative substitution”—refers to the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. [Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gln.

“Chemically modified”—when referring to the product of the invention, means a product (protein) where at least one of its amino acid resides is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

“Biologically active”—refers to the variant product having some sort of biological activity, for example, some physiologically measurable effect on target cells, molecules or tissues. In addition, this term refers to TL or TH product having sort of physiological activity, although the activity may be different than the original thrombopoietin or the original transport protein, to which these proteins show alignment.

“Immunologically active” defines the capability of a natural, recombinant or synthetic varient product, TL product or TH product or any fragment thereof, to induce a specific immune response in appropriate animals or cells and to bind with specific antibodies. Thus, for example, an immunologically active fragment of valiant product denotes a fragment which retains some or all of the immunological properties of the variant product, e.g can bind specific anti-variant product antibodies or which can elicit an immune response which will generate such antibodies or cause proliferation of specific immune cells which produce valiant.

“Optimal alignment”—is defined as an alignment giving the highest percent identity score. Such alignment can be performed using a variety of commercially available sequence analysis programs, such as the local alignment program LALIGN using a ktup of 1, default parameters and the default PAM. A preferred alignment is the one performed using the CLUSTAL-W program from MacVector (TM), operated with an open gap penalty of 10.0, an extended gap penalty of 0.1, and a BLOSUM similarity matrix. If a gap needs to be inserted into a first sequence to optimally align it with a second sequence, the percent identity is calculated using only the residues that are paired with a corresponding amino acid residue (i.e., the calculation does not consider residues in the second sequences that are in the “gap” of the first sequence). In case of alignments of known gene sequences with that of the new variant, the optimal alignment invariably included aligning the identical pails of both sequences together, then keeping apart and unaligned the sections of the sequences that differ one from the other.

“Having at least % identity”—with respect to two amino acid or nucleic acid sequence sequences, refers to the percentage of residues that are identical in the two sequences when the sequences are optimally aligned. Thus, 90% amino acid sequence identity means that 90% of the amino acids in two or more optimally aligned polypeptide sequences are identical, 70% identity 70% homology, etc. However this definition, where relating to variants of alternative splicing, explicitly excludes sequences which are 100% identical with the original sequence from which the variant of the invention was varied.

“Isolated nucleic acid molecule having a variant/TL/TH nucleic acid sequence”—is a nucleic acid molecule that includes the nucleic acid sequence for any of the above. Said isolated nucleic acid molecule may include any of the above nucleic acid sequence as an independent insert; may include any of the above nucleic acid sequence fused to an additional coding sequences, encoding together a fusion protein in which any of the above coding sequence is the dominant coding sequence (for example, the additional coding sequence may code for a signal peptide); or any of the above nucleic acid sequence may be in combination with non-coding sequences, e.g., introns or control elements, such as promoter and terminator elements or 5′ and/or 3′ untranslated regions, effective for expression of the coding sequence in a suitable host; or may be a vector in which any of the above coding sequences is a heterologous.

“Expression vector”—refers to vectors that have the ability to incorporate and express heterologous DNA fragments in a foreign cell. Many prokaryotic and eukaiyotic expression vectors are known and/or commercially available. Selection of appropriate expression vectors is within the knowledge of those having skill in the art.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

“Antibody”—refers to IgG, IgM, IgD, IgA, or IgG antibody. The definition includes polyclonal antibodies or monoclonal antibodies. This term refers to whole antibodies or fragments of the antibodies comprising the antigen-binding domain of the anti-variant product antibodies, e.g. antibodies without the Fc portion, single chain antibodies, fragments consisting of essentially only the variable, antigen-binding domain of the antibody, etc.

“Distinguishing antibody”—an antibody capable of binding to the variant product and not the original amino acid sequence from which it has been varied, or an antibody capable of binding to the original nucleic acid sequence and not to the variant production.

“Activator”—as used herein, refers to a molecule which mimics the effect of the natural variant product, the TL product or the TH products, or at times even increases or prolongs the duration of the biological activity of said products, as compared to that induced by the product without the activation. The mechanism may be by any mechanism known to prolonging activities of biological molecules such as binding to receptors; prolonging the lifetime of the molecules; increasing the activity of the molecules on its target; increasing the affinity of molecules to its receptor; inhibiting degradation or proteolysis of the molecules, or mimicking the biological activity of the variants on their targets, etc. Activators may be polypeptides, nucleic acids, carbohydrates, lipids, or derivatives thereof, or any other molecules which can bind to and activate the valiant product.

“Deactivator” or (“Inhibitor”)—refers to a molecule which modulates the activity of the valiant product, the TL product or the TH product in an opposite manner to that of the activator, by decreasing or shortening the duration of the biological activity of any of these products. This may be done by any mechanism known to deactivate or inhibit biological molecules such as block of the receptor, block of active site, competition on binding site in target, enhancement of degradation, etc. Deactivators may be polypeptides, nucleic acids, carbohydrates, lipids, or derivatives thereof, or any other molecules which bind to and modulate the activity of said product.

“Treating a disease”—refers to administering a therapeutic substance effective to ameliorate symptoms associated with a disease, to lessen the severity or cure the disease, or to prevent the disease from occurring.

“Detection”—refers to a method of detection of a disease, disorder, pathological or normal condition. This term may refer to detection of a predisposition to a disease as well as for establishing the prognosis of the patient by determining the severity of the disease.

“Probe”—the valiant nucleic acid sequence, a TL nucleic acid sequence or a TH nucleic acid sequence or a sequence complementary therewith, when used to detect presence of other similar sequences in a sample. The detection is carried out by identification of hybridization complexes between the probe and the assayed sequence. The probe may be attached to a solid support or to a detectable label.

“Original sequence”—in the context of the variant sequence, refers to the sequence from which the variants of the invention have been varied as a result of alternative slicing. This term referring to the amino acid sequence will also be denoted as “original peptide”.

### SUMMARY OF THE INVENTION

By one aspect the present invention is based on the finding of several novel, naturally occuitmg splice variants, which are naturally occurring sequences obtained by alternative splicing of known genes. The novel splice variants of the invention are not merely truncated forms, fragments or mutations of known genes, but rather novel sequences which naturally occur within the body of individuals.

The term “alternative splicing” in the context of the present invention and claims refers to: intron inclusion, exon exclusion, addition or deletion of terminal sequences in the variant as compared to the original sequences, as well as to the possibility of “intron retention”. Intron retention is an intermediate stage in the processing of RNA transcripts, where prior to production of fully processed mRNA the intron (naturally spliced in the original sequence) is retained in the variant. These intermediately processed RNAs may have physiological significance and are also within the scope of the invention.

The novel valiant products of the invention may have the same physiological activity as the original peptides from which they have been varied (although perhaps at a different level); may have an opposite physiological activity from the activity featured by the original peptides from which they are varied; may have a completely different, unrelated activity to the activity of the original from which they are varied; or alternatively may have no activity at all and this may lead to various diseases or pathological conditions. The variants may differ from the original sequences by property or properties not connected to physiological activities such as: clearance rate, turn-over time, resistance to degradation, affinity of interactions with co-factor, tissue distribution, expression patterns, etc.

The novel variants may also serve for detection purposes, i.e. their presence or level may be indicative of a disease, disorder, pathological or normal condition or alternatively the ratio between the level variants and the level original sequence from which they were varied (either at the mRNA level or the amino acid sequence level), or the ratio to other variants may be indicative to a disease, disorder, pathological or normal condition.

For example, for detectional purposes, it is possible to establish differential expression of various variants in various tissues. A certain variant may be expressed mainly in one tissue, while the original sequence from which it has been varied, or another variant may, be expressed mainly in another tissue. Understanding of the distribution of the variants in various tissues may be helpful in basic research, for understanding the physiological function of the genes as well as may help in targeting pharmaceuticals or developing pharmaceuticals.

The study of the variants may also be helpful to distinguish various stages in the life cycles of the same type of cells which may also be helpful for development of pharmaceuticals for various pathological conditions in which cell cycles is non-normal, notably cancer.

Thus the detection may by determination of the presence or the level of expression of the valiant within a specific cell population, comprising said presence or level between various cell types in a tissue, between different tissues and between individuals.

By a second aspect, the present invention is based on the surprising finding that there exists in humans two novel homologs (each a result of alternative splicing of a new gene) of the Thrombopoietin (herein after: TL) having a significant homology to this protein. The novel TL is homolog to the known thrombopoietin in the erythropoietin-like N-terminal domain.

By a third aspect, the present invention is based on the surprising finding of novel molecules which feature homology to several transporter molecules including the vesicular transporter molecules of biogenic amines, for example the momoamine transporters, vesicular acetylcholine transporters and other neurotransmitter transporters, as well as to several transporters of sugars.

Thus the present invention provides by its first aspect (variant sequences), a novel isolated nucleic acid molecule comprising or consisting of any one of the coding sequence SEQ ID NO: 1 to SEQ ID NO: 28, fragments of said coding sequence having at least 20 nucleic acids (provided that said fragments are continuous stretches of nucleotides not present in the original sequence from which the variant was varied), or a molecule comprising a sequence having at least 90%, identity to SEQ ID NO: 1 to SEQ ID NO: 28, provided that the molecule is not completely identical to the original sequence from which the variant was varied.

The present invention further provides a protein or polypeptide comprising or consisting of an amino acid sequence encoded by any of the above nucleic acid sequences, termed herein “variant product”, for example, an amino acid sequence having the sequence as depicted in any one of SEQ ID NO: 42 to SEQ ID NO: 69, fragments of the above amino acid sequence having a length of at least 10 amino acids coded by the above fragments of the nucleic acid sequences, as well as homologues of the above amino acid sequences in which one or more of the amino acid residues has been substituted (by conservative or non-conservative substitution) added, deleted, or chemically modified.

The deletions, insertions and modifications should be in regions, or adjacent to regions, wherein the variant differs from the original sequence.

For example, where the valiant is different from the original sequence by addition of a short stretch of 10 amino acids, in the terminal or non-terminal portion of the peptide, the invention also concerns homologues of that valiant where the additional short stretch is altered for example, it includes only 8 additional amino acids, includes 13 additional amino acids, or it includes 10 additional amino acids, however some of them being conservative or non-conservative substitutes of the original additional 10 amino acids of the novel variants. In all cases the changes in the homolog, as compared to the original sequence, are in the same regions where the valiant differs from the original sequence, or in regions adjacent to said region.

Another example is where the variant lacks a non-terminal region (for example of 20 amino acids) which is present in the original sequence (due for example to exon exclusion). The homologues may lack in the same region only 17 amino acids or 23 amino acids. Again the deletion is in the same region where the variant lacks a sequence as compared to the original sequence, or in a region adjacent thereto.

It should be appreciated that once a man versed in the art's attention is directed to the importance of a specific region, due to the fact that this region differs in the variant as compared to the original sequence, there is no problem in derivating said specific region by addition to it, deleting from it, or substituting some amino acids in it. Thus homologues of variants which are derivated from the variant by changes (deletion, addition, substitution) only in said region as well as in regions adjacent to it are also a part of the present invention. Generally, if the variant is distinguished from the original sequence by some sort of physiological activity, then the homolog is distinguished from the original sequence in essentially the same manner.

The present invention provides by the second of its aspects, a novel isolated nucleic acid molecule comprising of or consisting of the nucleic acid sequence of any one of SEQ ID NO: 29 or SEQ ID NO: 30, fragment of the sequence having at least 20 nucleic acids, or a molecule comprising a sequence having at least 70%, preferably 80%, and most preferably 90% identity to any one of SEQ ID NO: 29 or 30. The present invention further provides a protein or a polypeptide comprising or consisting of an amino acid sequence encoded by any one of the above nucleic acid sequences, termed herein as “TL product”. For example, the amino acid sequence depicted in SEQ ID NO: 70 (since both SEQ ID NO: 29 and 30 code for the same amino acid sequence) fragments of the above amino acid sequence having a length of at least 10 amino acids as well as homolog of the amino acid sequence of SEQ ID NO: 70 in which one or more amino acid residues have been substituted, by conservative or non conservative substitution, added, deleted or chemically modified.

The present invention further provides by its third aspect a novel isolated nucleic acid molecule comprising or consisting of any of the nucleic acid sequence of SEQ ID NO: 31 to 41, fragments of said sequence having at least 20 nucleic acids, or a molecule comprising a sequence having at least 70%, preferably 80%, and most preferably 90% identity to any of SEQ ID NO: 31 to 41. The present invention further provides a protein or polypeptide comprising or consisting of an amino acid sequence encoded by any of the above nucleic acid sequences, termed hereinafter: “TH product”. For example, an amino acid sequence having a sequence as depicted in any one of SEQ ID NO: 71 to 81, fragments of the above amino acid sequence having a length of at least 10 amino acids, as well as homologs of the amino acid sequences SEQ ID NO: 71 to SEQ ID NO: 81 in which one or more of the amino acid residues have been substituted (by conservative or non conservative substitution) added, deleted, or chemically modified.

The present invention further provides nucleic acid molecule comprising or consisting of a sequence which encodes the above valiant, TL and TH amino acid sequences, (including the fragments and homologues of the amino acid sequences). Due to the degenerative nature of the genetic code, a plurality of alternative nucleic acid sequences, beyond those depicted in any one of SEQ ID NO: 42 to SEQ ID NO: 81, can code for any of the amino acid sequence of the invention (variants, TL and TH). Those alternative nucleic acid sequences which code for the same amino acid sequences codes by the sequence SEQ ID NO: 1 to SEQ ID NO: 41 are also an aspect of the of the present invention.

The present invention further provides expression vectors and cloning vectors comprising any of the above nucleic acid sequences, as well as host cells transected by said vectors.

The present invention still further provides pharmaceutical compositions comprising, as an active ingredient, said nucleic acid molecules, said expression vectors, or said protein or polypeptide.

These pharmaceutical compositions are suitable for the treatment of diseases and pathological conditions, which can be ameliorated or cured by raising the level of any one of the valiant products of the invention, by raising the level of the TL product or by raising the level of TH products of the invention. The diseases in connection with the variants of the invention are as explained below in Example I.

The diseases in connection with the TL aspect are, for example, thrombocytopenia, a reduction in clot-inducing platelets, which occur in cancer patients treated with chemotherapy or a condition of delayed platelet recovery after hematopoietic stem cell transplantation.

The diseases in connection with the TH are those which can be ameliorated, cured or prevented by raising the level of the TH product. Typically these are diseases which are manifested by non-normal levels of transport of various ligands and/or by non-normal levels of secretion of various ligands. Alternatively, the diseases may be due to conditions in which the level of the transport is normal, but a therapeutically beneficial effect may be achieved by raising the level of the transport protein or by regulating its activity. For example, diseases where it is desired to modulate the release or uptake of secreted substances such as neurotransmitters and sugar.

For example, where the ligand is neurotransmitters (neurotransmitters as choline) and the biogenic amines (low molecular weight neurotransmitter substances such as dopamine, norepinephrine, epinephrine, serotonin and histamine) the diseases may be such in which a beneficial effect may be achieved by regulation of the secretion of neurotransmitters (which can be non-normal levels of secretion or normal levels of secretion), and this may include pathological conditions involved with substance abuse (such as cocaine or other drug abuse), diseases which involve spasmic movement due to unregulated neuronal firing; schizophrenia, dementia and other neuro degenerative diseases; depression and epilepsy as well as diseases involved in non-normal transport of sugars through membranes, such as various types of diabetes.

The TH product of the present invention may also be used in conjunction with imaging substances for detection and imaging purposes and may be used either as a target to which imaging substances bind and this binds to the membranes (for example neurotransmitter vesicles' membranes, or sugar transporting membranes) or alternatively the products themselves may be used to transport imaging substances, which mimick the natural ligands (neurotransmitters as choline and the biogenic amines low molecular weight neurotransmitter substances such as dopamine, norepinephrine, epinepluine, serotonin and histamine or sugars) in their binding to the product and thus are transferred by the product of the invention across membranes for imaging purposes.

By another embodiment, the present invention provides a nucleic acid molecule comprising or consisting of a non-coding sequence which is complementary to that of any one of SEQ ID NO: 1 to SEQ ID NO: 28, or complementary to a sequence having at least 90% identity to said sequence (with the proviso added above) or a fragment of said two sequences (according to the above definition of fragment).

By this second embodiment, the present invention provides a nucleic acid molecule comprising or consisting of a non coding sequence which is complementary to that of any one of SEQ ID NO: 29 or 30, or complementary to a sequence having at least 70% identity to said sequence or fragment of said two sequences.

In addition, according to the third aspect, the present invention provides a nucleic acid molecule comprising or consisting of a non coding sequence which is complementary to any of the sequences of SEQ ID NO: 31 to SEQ ID NO: 41, or complementary to a sequence having at least 70% identity with said sequence, or fragment of said two sequences.

The complementary sequence may be a DNA sequence which hybridizes with any one of SEQ of ID NO: 1 to SEQ ID NO: 41 or hybridizes to a portion of that sequence having a length sufficient to inhibit the transcription of the complementary sequence. The complementary sequence may be a DNA sequence which can be transcribed into an mRNA being an antisense to the mRNA transcribed from any one of SEQ ID NO: 1 to SEQ ID NO: 41 or into an mRNA which is an antisense to a fragment of the mRNA transcribed from any one of SEQ ID NO: 1 to SEQ ID NO: 41 which has a length sufficient to hybridize with the mRNA transcribed from SEQ ID NO: 1 to SEQ ID NO: 41, so as to inhibit its translation. The complementary sequence may also be the mRNA or the fragment of the mRNA itself.

The complementary nucleic acids according to the first, second and third aspects of the invention may be used for therapeutic or diagnostic applications for example as probes used for the detection of the variants, TL or TH sequences of the invention. The presence of the variant transcript, TL or TH or the level of the variant transcript, TL or TH transcripts may be indicative of a multitude of diseases, disorders and various pathological as well as normal conditions. In addition or alternatively, the ratio of the level of the transcripts of the variants of the invention may also be compared to that of the transcripts of the original sequences from which have been varied, or to the ratio of the level of transcript of other variants, and said ratio may be indicative to a multitude of diseases, disorders and various pathological and normal conditions.

As regards the TL transcript, the level of each of the alternative splice variants depicted in SEQ ID NO: 29 or 30, to each other may also be indicative to a plurality of diseases.

As regards the TH transcripts, the level or ratio of each of the alternative splice variants of SEQ ID NO: 31 to 41 or the ratio to each other; may also be indicative to a multitude of diseases, disorders and various pathological and normal conditions.

The present invention also provides expression vectors comprising any one of the above defined complementary nucleic acid sequences and host cells transfected with said nucleic acid sequences or vectors, being complementary to those specified in the first aspect of the invention.

The invention also provides anti-valiant product antibodies, anti-TL product antibodies and anti-TH product antibodies, namely antibodies directed against the variant product which specifically bind to said variant product. Said antibodies are useful both for diagnostic and therapeutic purposes. For example said antibodies may be as an active ingredient in a pharmaceutical composition as will be explained below.

The present invention also provides pharmaceutical compositions comprising, as an active ingredient, the nucleic acid molecules which comprise or consist of said complementary sequences, or of a vector comprising said complementary sequences. The pharmaceutical composition thus provides pharmaceutical compositions comprising, as an active ingredient, said anti-variant product antibodies, said anti-TL product antibodies or said anti-MTPH product antibodies.

The pharmaceutical compositions comprising said anti-variant product antibodies, anti-TL product antibodies or anti-TH antibodies or the nucleic acid molecule comprising said complementary sequence, are suitable for the treatment of diseases and pathological conditions where a therapeutically beneficial effect may be achieved by neutralizing the variant, TL or TH (either at the transcript or product level) or decreasing the amount of the variant product, TL or TH product or blocking its binding to its target, for example, by the neutralizing effect of the antibodies, or by the effect of the antisense mRNA in decreasing the expression level of the variant TL or TH sequences).

According to another embodiment of the invention the present invention provides methods for detecting the level of the transcript (mRNA) of said variant product, TL product or TH in a body fluid sample, or in a specific tissue sample, for example by use of probes comprising or consisting of said coding sequences (and determing level of hybridization of the proteins) or by using any amplification method utility suitable primers; as well as methods for detecting levels of expression of said product in tissue, e.g. by the use of antibodies capable of specifically reacting with the variant products of the invention. Detection of the level of the expression of the valiant of the invention in particular as compared to that of the original sequence from which it was varied or compared to other valiant sequences all varied from the same original sequence may be indicative of a plurality of physiological or pathological conditions.

The method, according to this latter aspect, for detection of a nucleic acid sequence which encodes the valiant product, the TL product or the TH in a biological sample, comprises the steps of:

(a) providing a probe comprising at least one of the nucleic acid sequences defined above (SEQ ID NO: 1 to SEQ ID NO: 41);

(b) contacting the biological sample with said probe under conditions allowing hybridization of nucleic acid sequences thereby enabling formation of hybridization complexes;

(c) detecting hybridization complexes, wherein the presence of the complexes indicates the presence of nucleic acid sequence encoding the valiant product, the TL product or the TH in the biological sample.

The method as described above is qualitative, i.e. indicates whether the transcript is present in or absent from the sample. The method can also be quantitative, by determining the level of hybridization complexes and then calibrating said levels to determining levels of transcripts of the desired variant, TL or TH in the sample.

Both qualitative and quantitative determination methods can be used for diagnostic, prognostic and therapy planning purposes.

By a preferred embodiment the probe is part of a nucleic acid chip used for detection purposes, i.e. the probe is a part of an array of probes each present in a known location on a solid support.

The method, according to the same latter aspect, for detection of a nucleic acid sequence which encodes the valiant product, the TL product or the TH in a biological sample, comprises the steps of:

(I) contacting the sample with primers for amplification of any one of SEQ ID NO: 1 to SEQ ID NO: 41;

(II) proving reagents for amplification;

(III) detecting the presence of amplified products,

Said products indicating the presence of the nucleic acid in the sample.

The nucleic acid sequence used in the above method may be a DNA sequence an RNA sequence, etc; it may be a coding or a sequence or a sequence complementary thereto (for respective detection of RNA transcripts or coding-DNA sequences). By quantization of the level of hybridization complexes and calibrating the quantified results it is possible also to detect the level of the transcript in the sample.

Methods for detecting mutations in the region coding for the valiant, TL or TH product are also provided, which may be methods carried out in a binary fashion, namely merely detecting whether there is any mismatches between the normal variant, TL or TH nucleic acid sequence of the invention and the one present in the sample, or carried out by specifically detecting the nature and location of the mutation.

The present invention also concerns a method for detecting variant product, a TL product or TH product in a biological sample, comprising the steps of:

(a) contacting with said biological sample any of the antibodies of the invention, thereby forming an antibody-antigen complex; and

(b) detecting said antibody-antigen complexes

wherein the presence of said antibody-antigen complex correlates with the presence of variant product, TL product or TH product, respectively in said biological sample.

Many diseases are diagnosed by detecting the presence of antibodies against a protein characterizing the disease in the blood, serum or any other body fluid of the patient. The present invention also concerns a method for detecting anti-variant antibody in a biological sample, comprising:

(a) contacting said sample with the valiant product, TL product or a MTPH product of the invention, thereby forming an antibody-antigen complex; and

(b) detecting said antibody-antigen complex

wherein the presence of said antibody-antigen complex correlates with the presence of anti-valiant, anti-TL or anti-TH antibodies in the sample.

As indicated above, both methods (for detection of any one of the products and for detection of any one of the antibodies against the product) can be quantitized to determine the level or the amount of the variant or antibody in the sample, alone or in comparison to the level of the original amino acid sequence from which it was varied or compared to the level of antibodies against the original amino acid sequence, and qualitative and quantitative results may be used for diagnostic, prognostic and therapy planning purposes.

The invention also concerns distinguishing antibodies, i.e. antibodies capable of binding either to the variant product or to the original sequence from which the valiant has been varied, while not binding to the original sequence or the variant product respectively. These distinguishing antibodies may be used for detection purposes.

By yet another aspect the invention also provides a method for identifying candidate compounds capable of binding to the variant product, the TL product or the TH product and modulating their activity (being either activators or deactivators). The method includes:

(i) providing a protein or polypeptide comprising an amino acid sequence substantially as depicted in any one of SEQ ID NO: 42 to 81, or a fragment of such a sequence;

(ii) contacting a candidate compound with said amino acid sequence;

(iii) measuring the physiological effect of said candidate compound on the activity of the amino acid sequences and selecting those compounds which show a significant effect on said physiological activity.

The present invention also concerns compounds identified by the methods described above, which compound may either be an activator of the variant product, the TL product or the TH, or a deactivator thereof.

The detection may be for the same diseases which the pharmaceutical composition of the invention are referred as capable of treating.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1 to 28 show the alignment of any one of SEQ ID NO: 1 to SEQ ID NO: 28, respectively, to the original sequence from which they were varied.

FIG. 29 is the alignment of SEQ ID NO: 38 and SEQ ID NO: 39, i.e. of the two splice variants.

FIG. 30 is an alignment between SEQ ID NO: 78 and SEQ ID NO: 79, i.e. of the two splice variants.

FIG. 31 is an alignment between SEQ ID NO: 40 and SEQ ID NO: 41, i.e. of the two splice variants.

FIG. 32 is an alignment between SEQ ID NO: 80 and SEQ ID NO: 81, i.e. of the two splice variants.

FIG. 33 is an alignment between SEQ ID NO: 40 and TH sequence.

FIG. 34 is an alignment between SEQ ID NO: 41 and TH sequence.

FIG. 35 is an alignment between SEQ ID NO: 40 and a known protein (acession no. gi4887697).

FIG. 36 is an alignment between between SEQ ID NO: 40 and a known protein (acession no. gi4506987).

FIG. 37 is an alignment between SEQ ID NO: 41 and a known protein (acession no. gi4887697)

FIG. 38 is an alignment between SEQ ID NO: 41 and a known protein (acession no. gi4506987)FIG. 39 (A-H) shows a prediction of transmembrane domains of SEQ ID NO:80

FIG. 40 (A-H) shows a prediction of transmembrane domains of SEQ ID NO:81

FIG. 41 is a Northern Blot analysis of mRNA obtained from (A.) various brain regions or (B.) various human tissues, and tested with TH specific nucleotide probe.

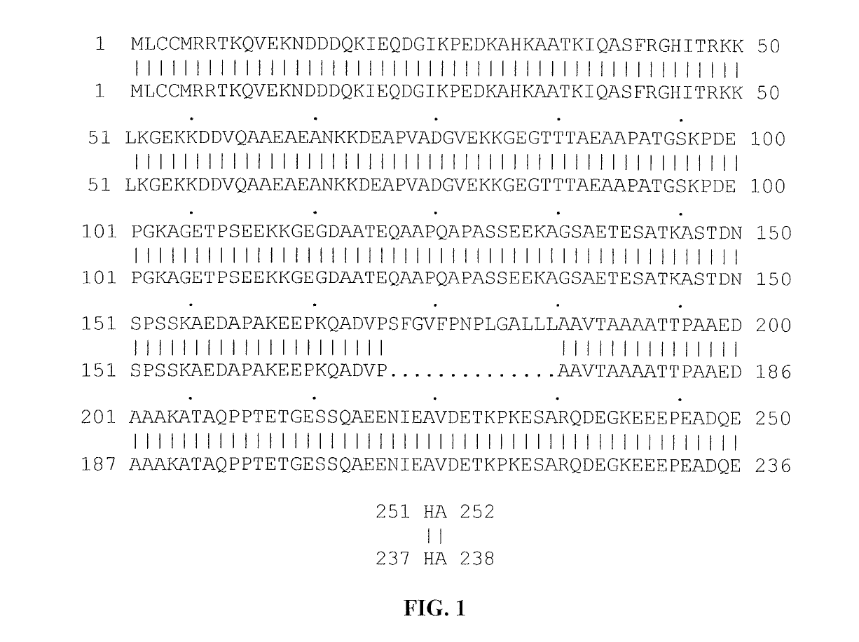

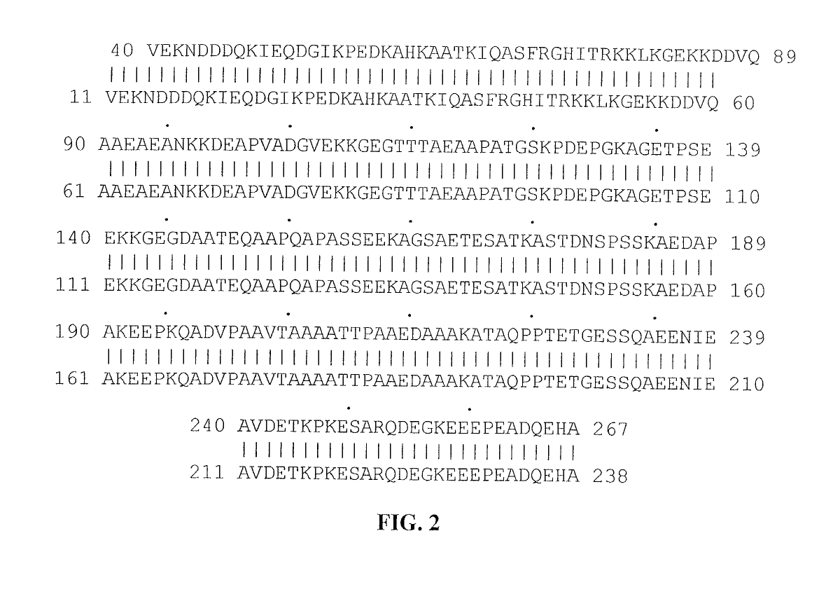

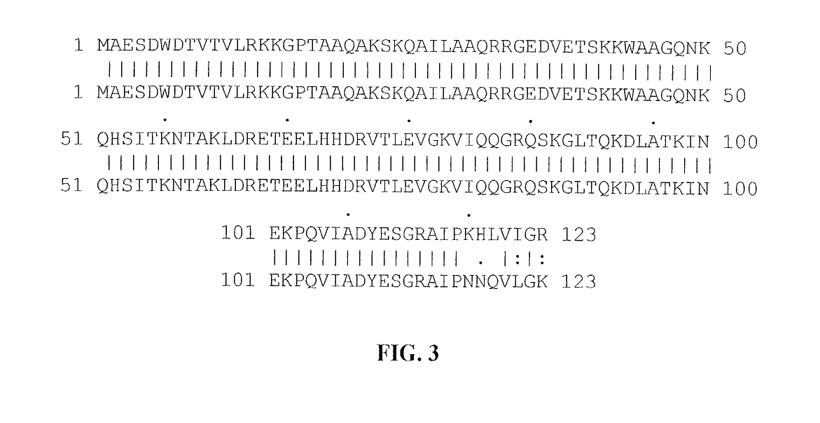

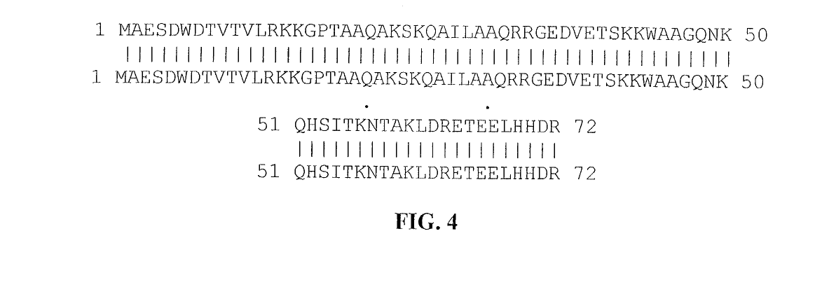

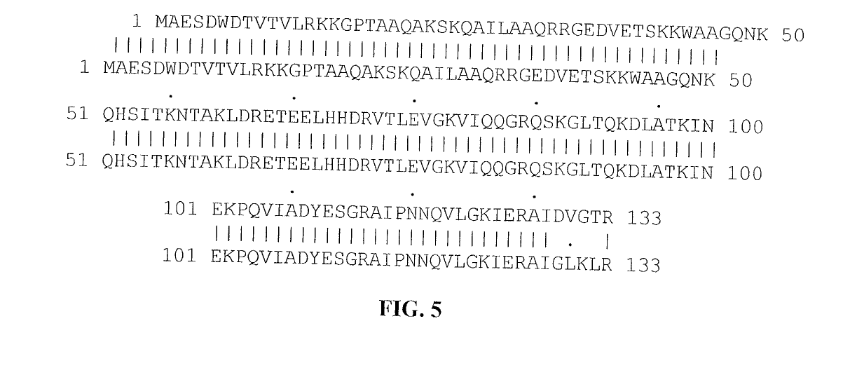

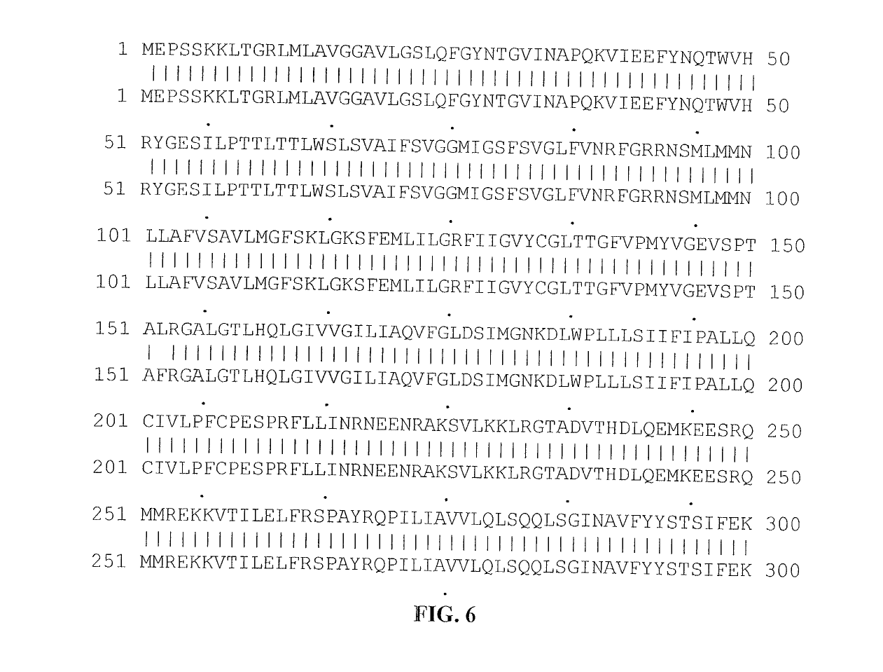

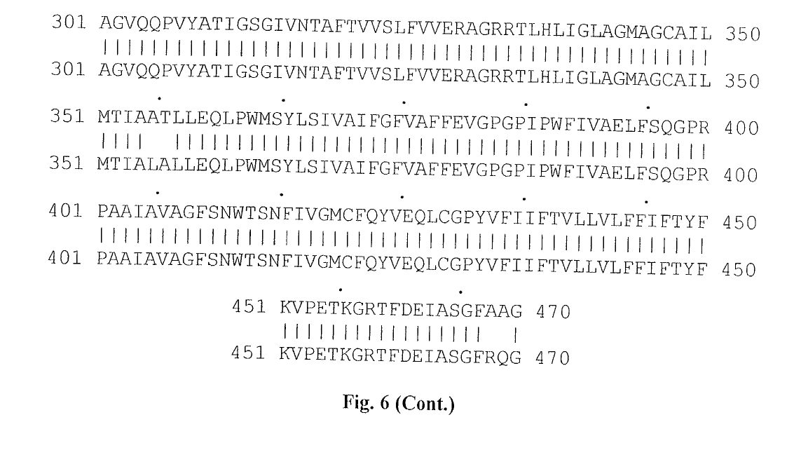

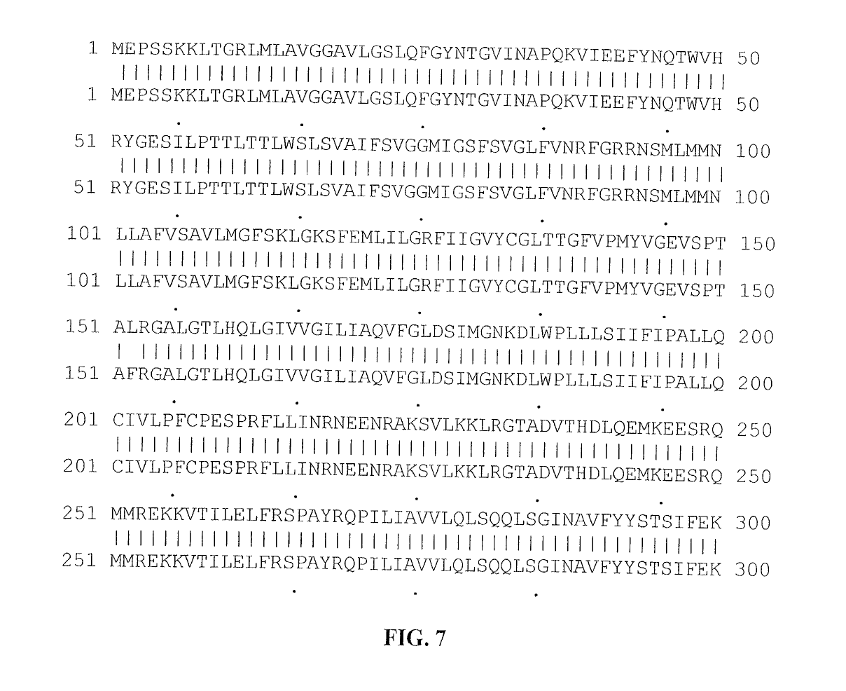

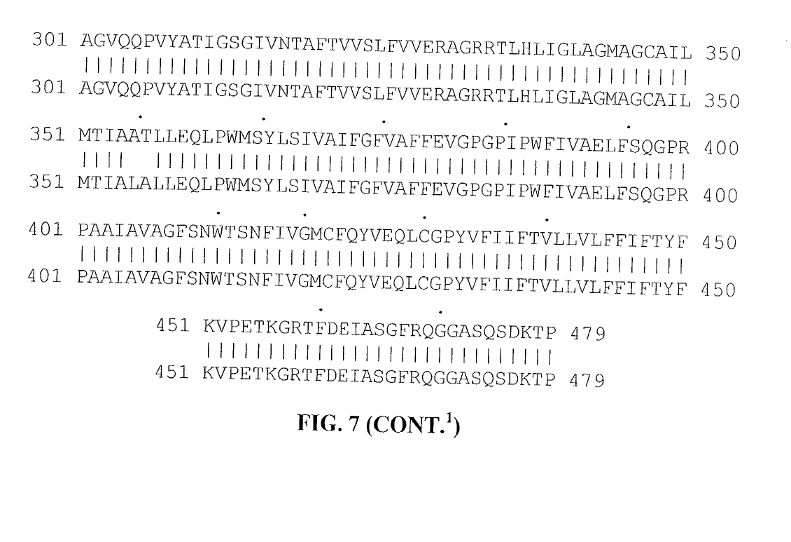

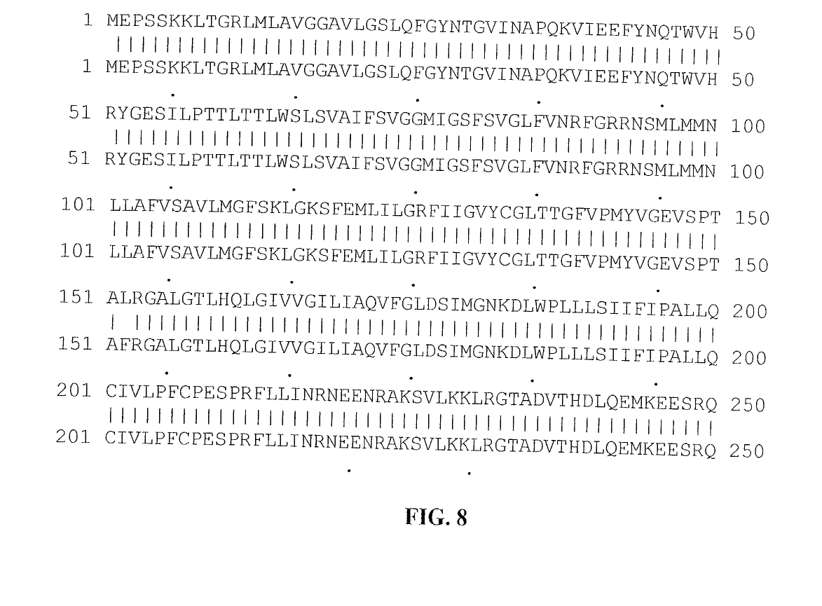

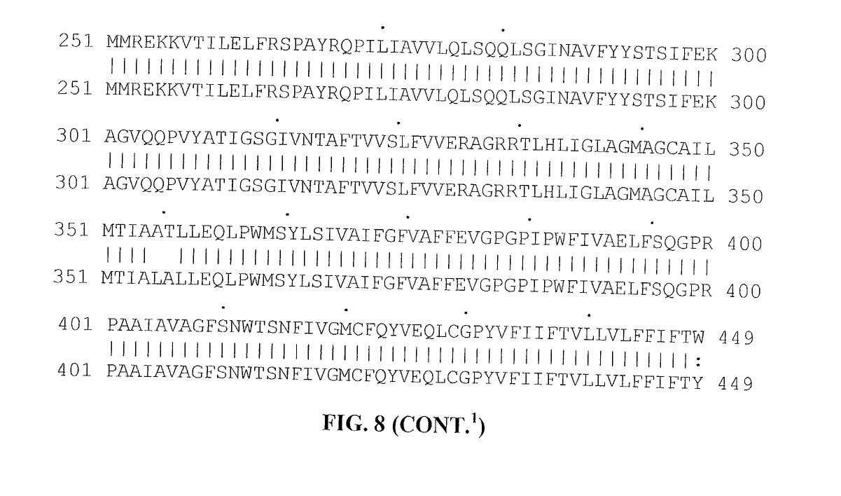

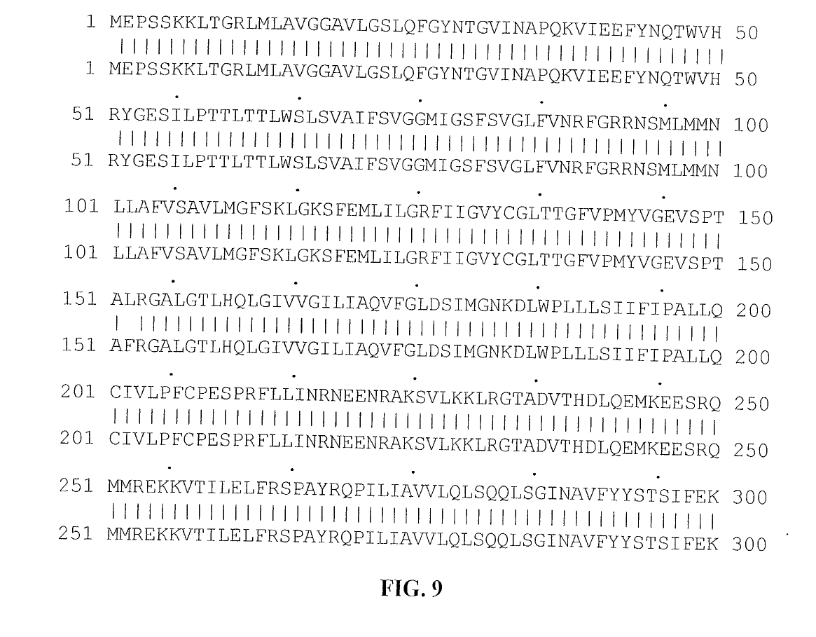

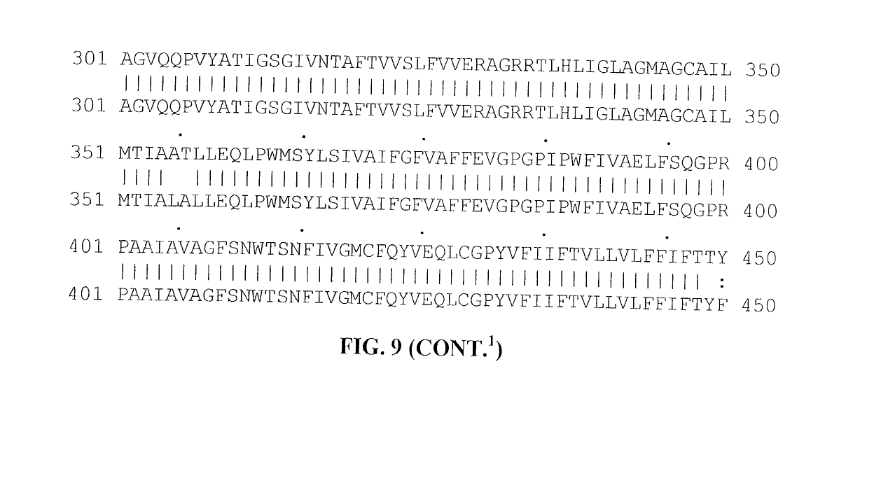

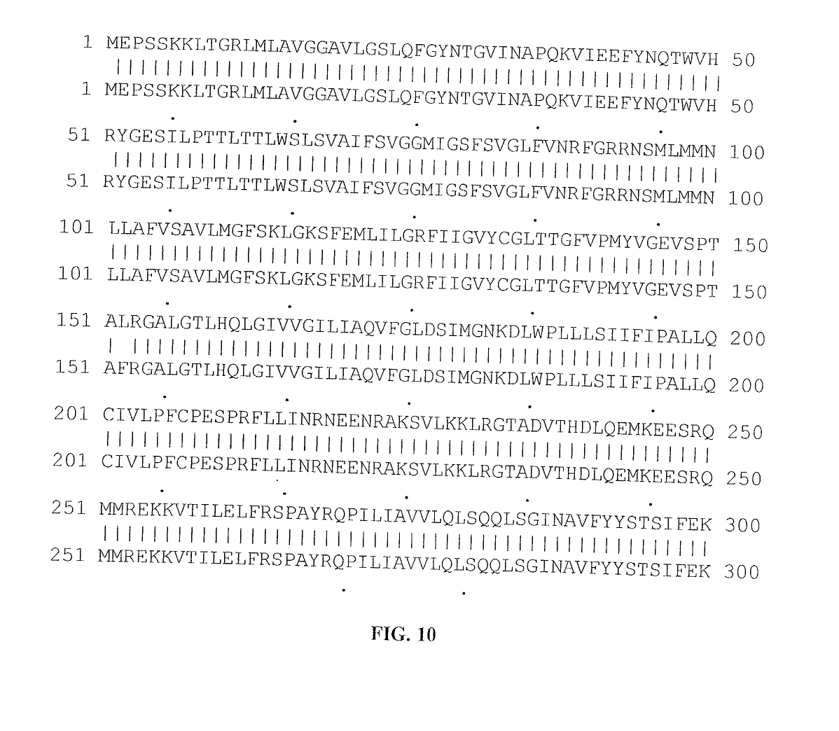

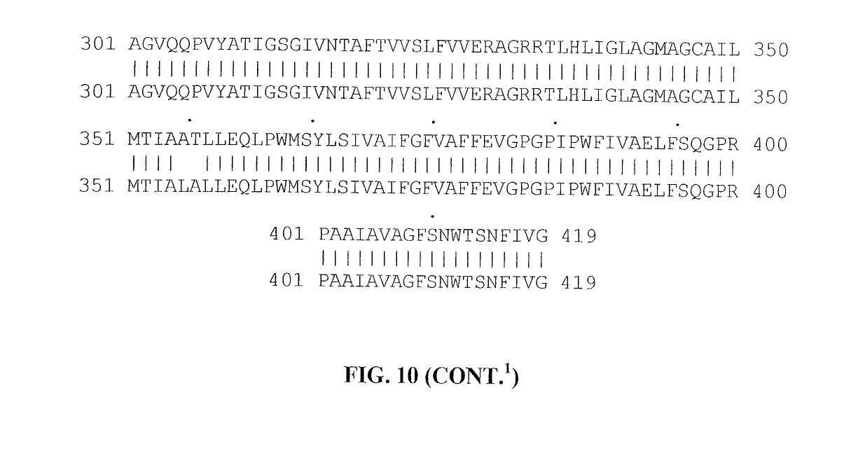

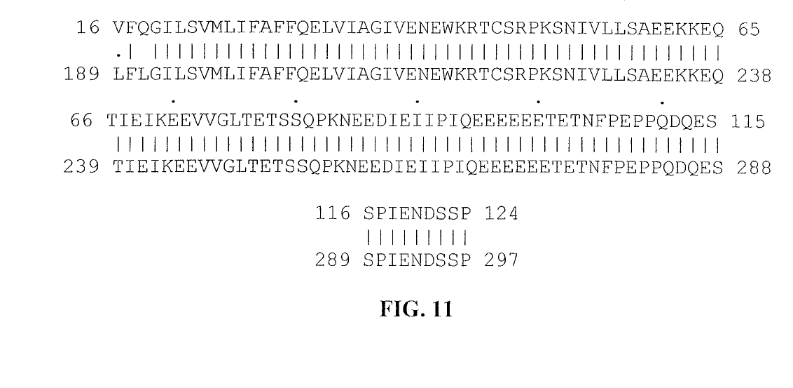

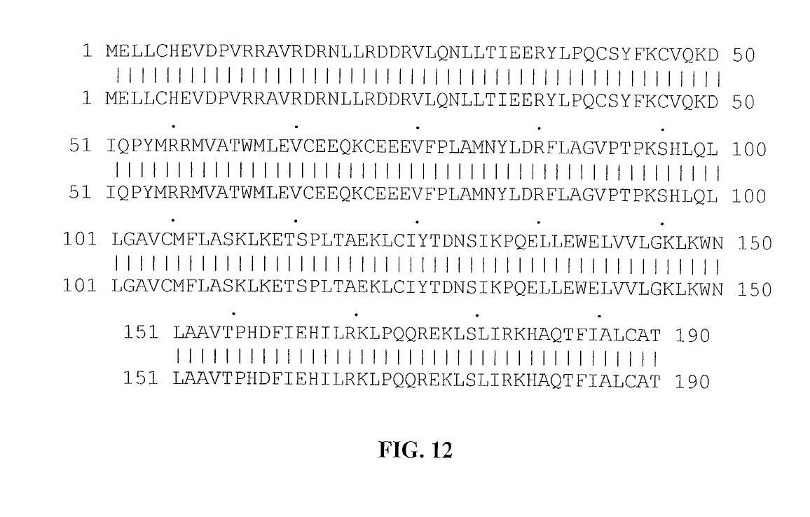

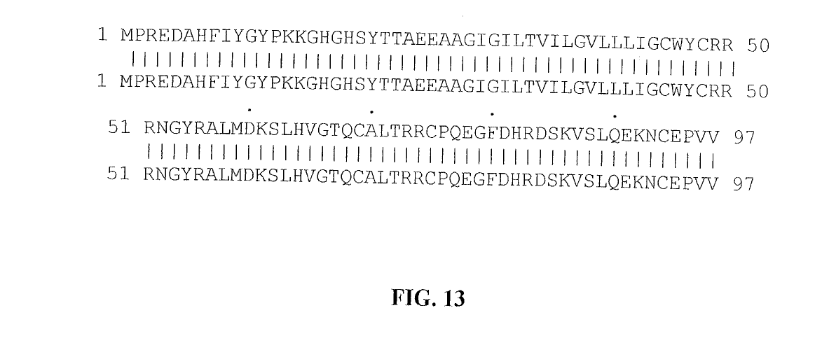

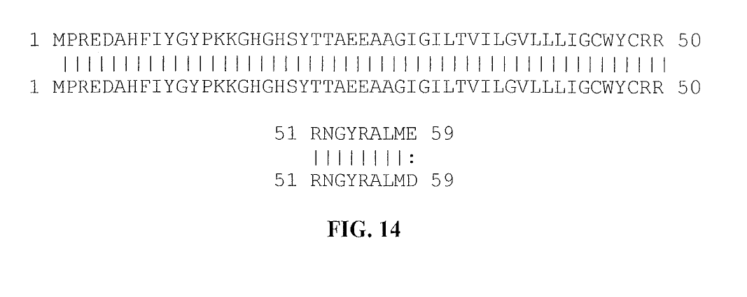

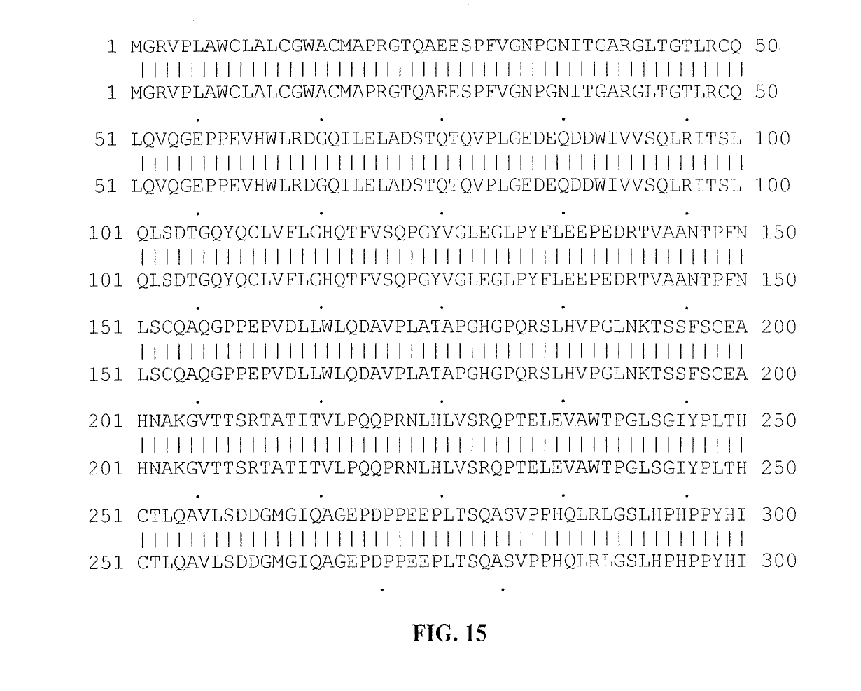

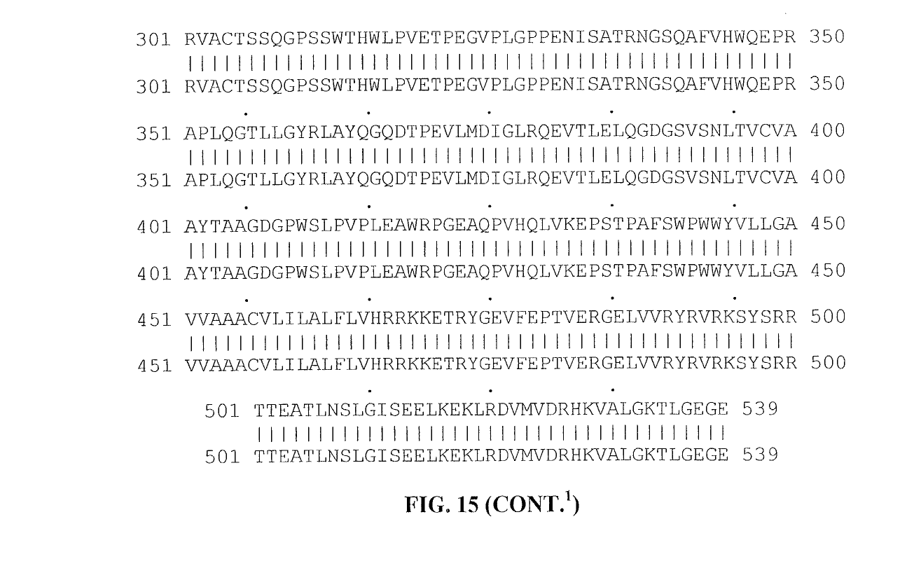

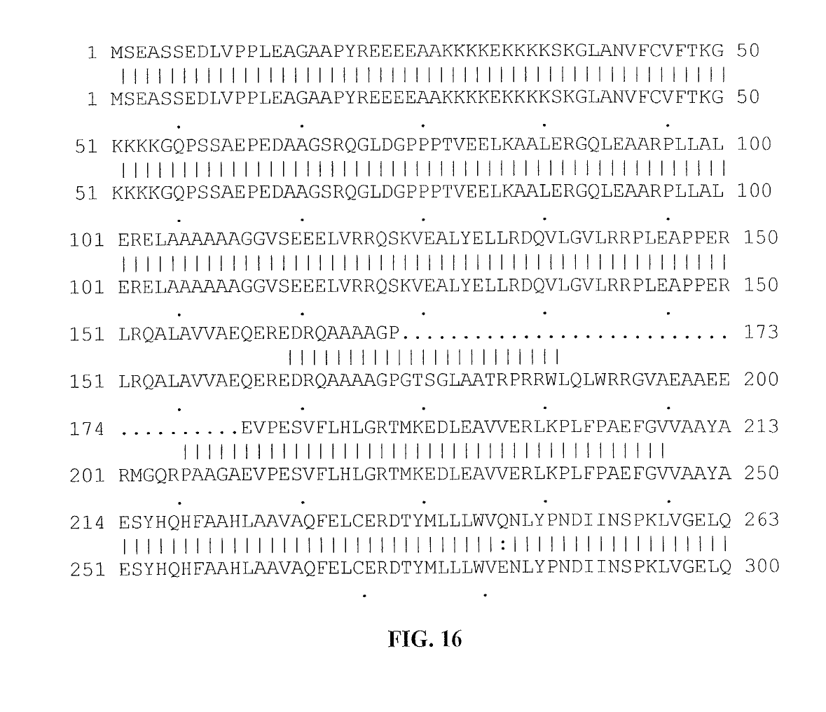

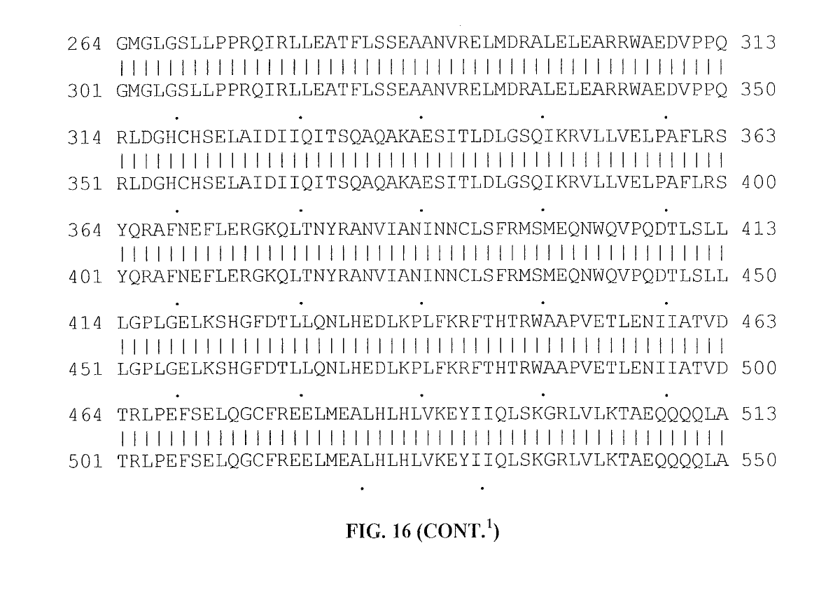

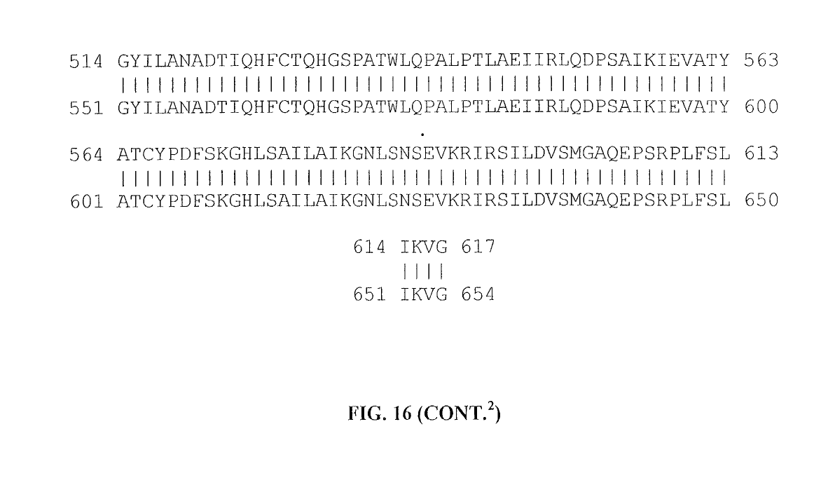

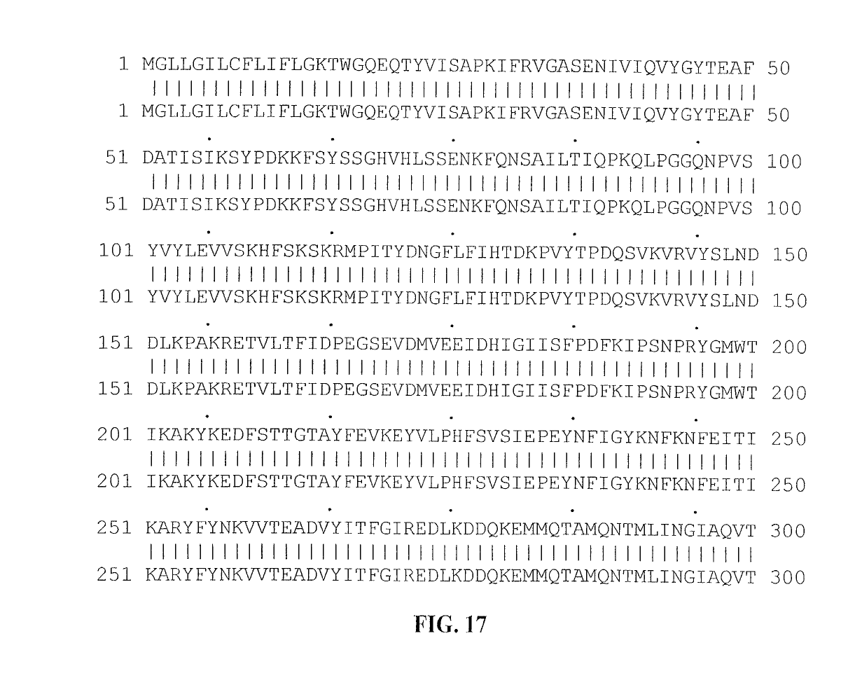

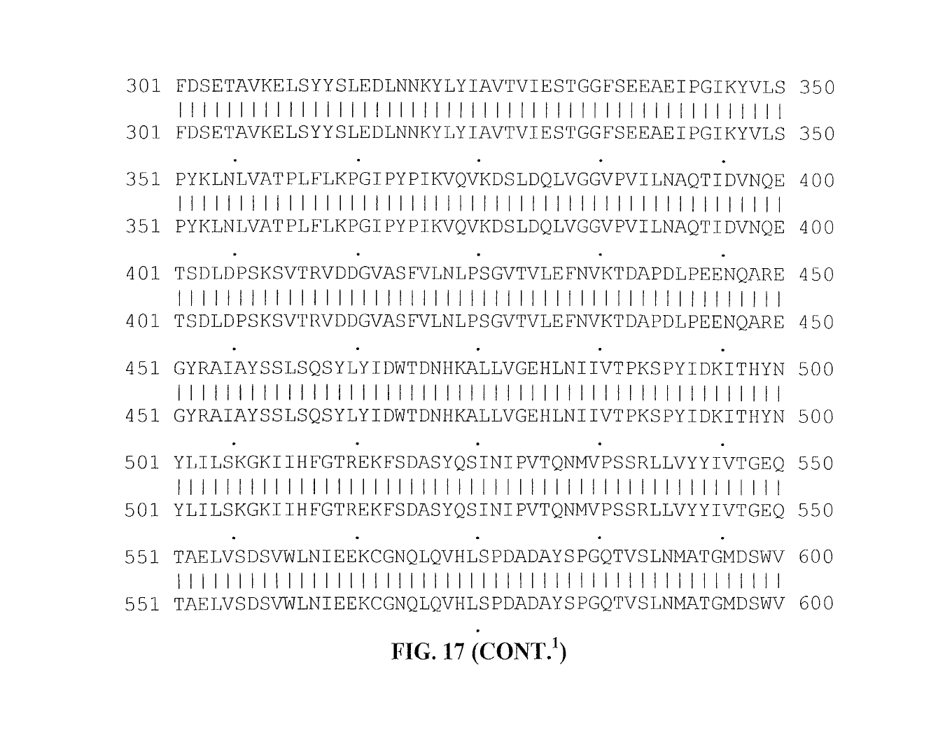

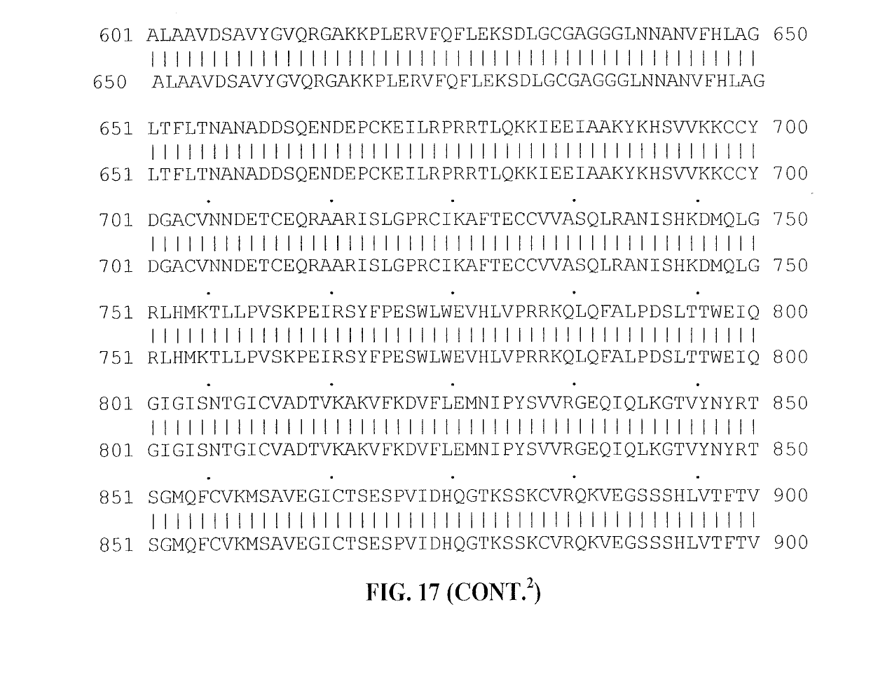

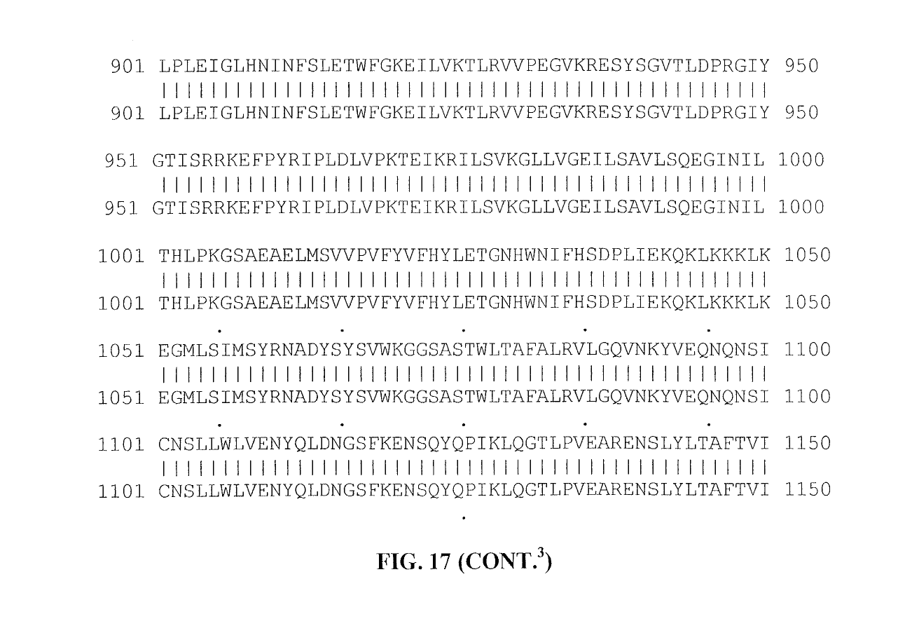

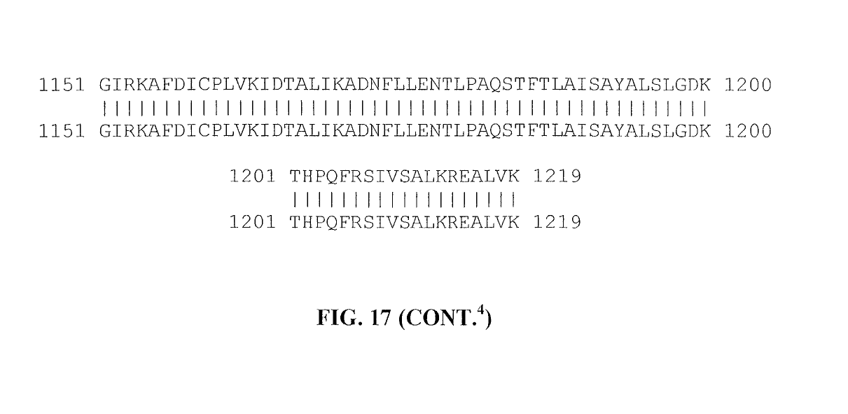

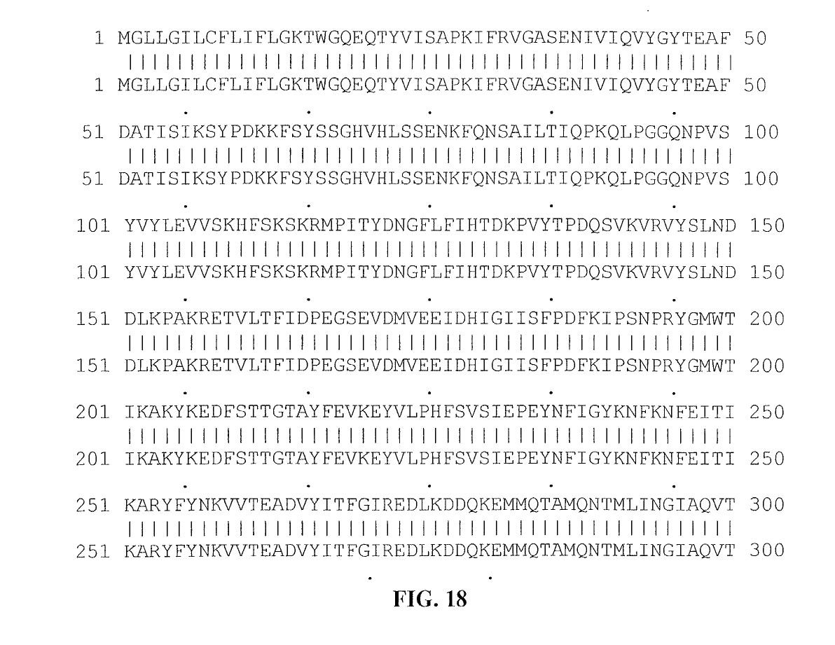

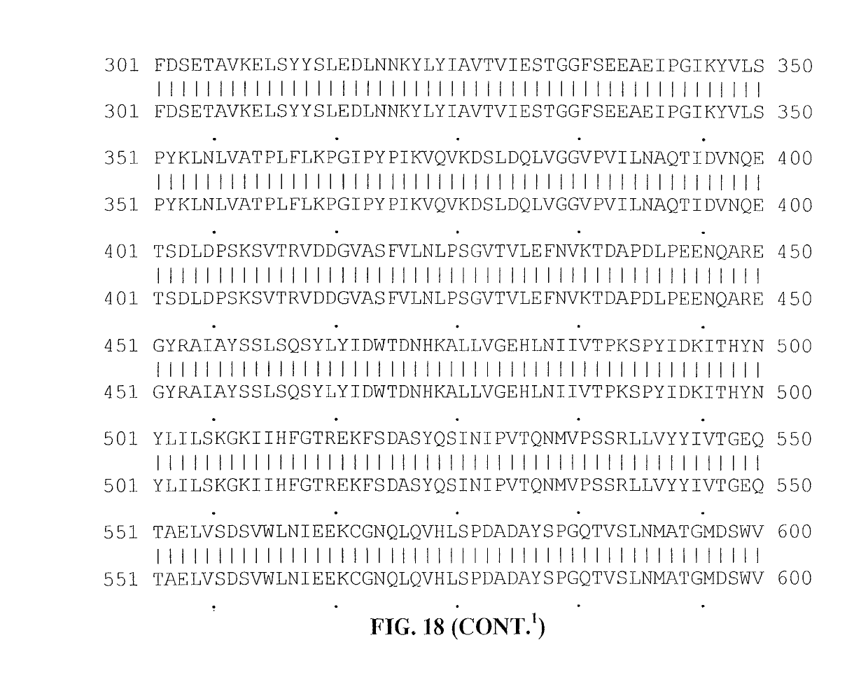

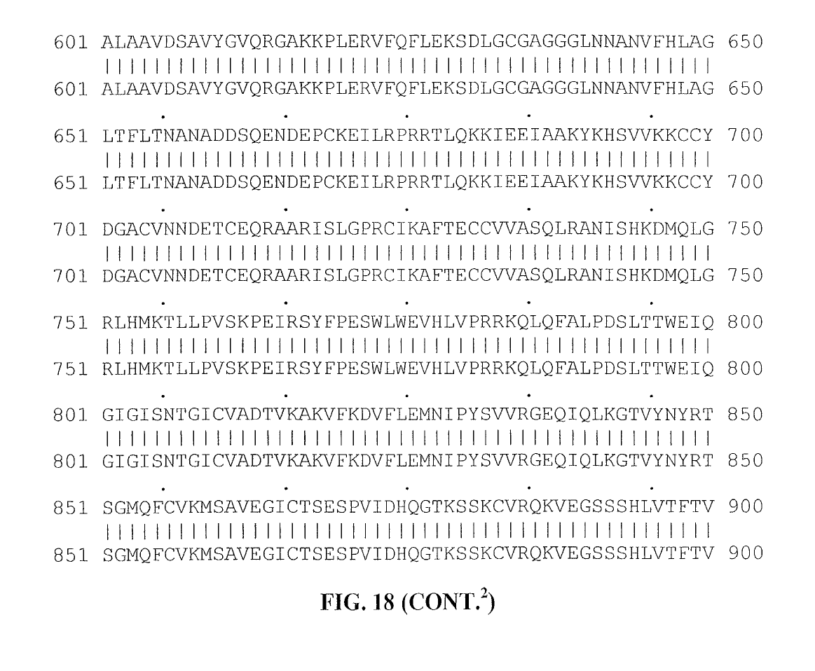

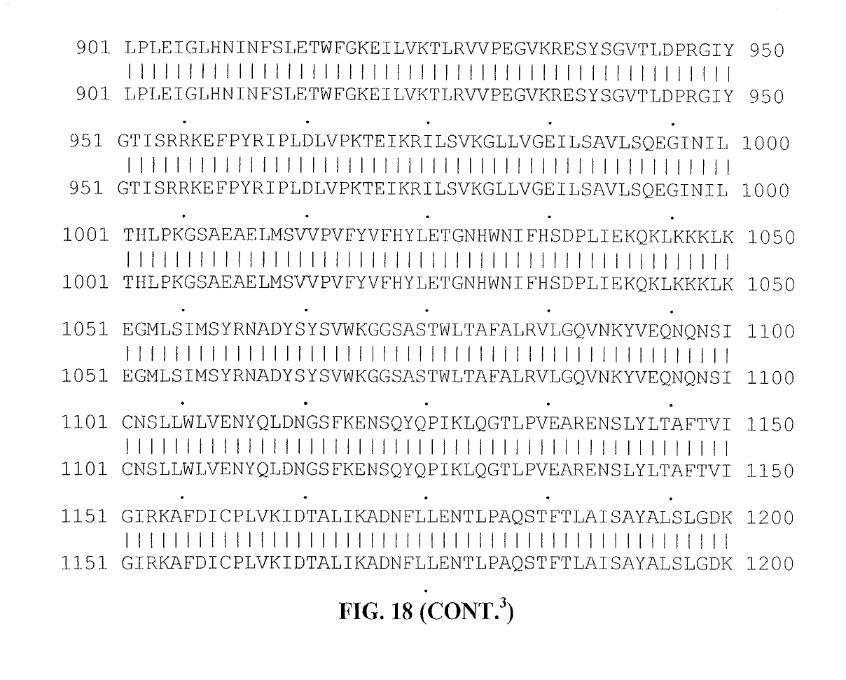

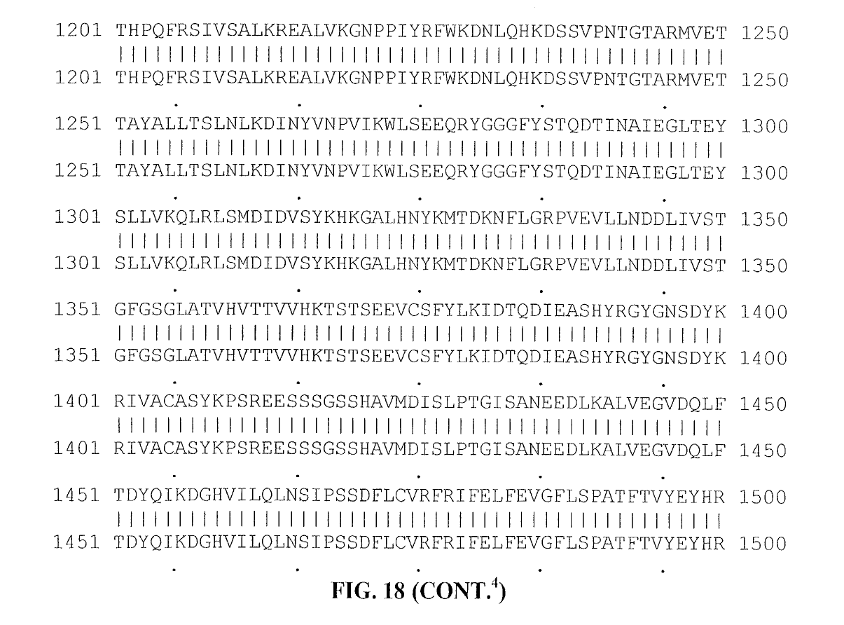

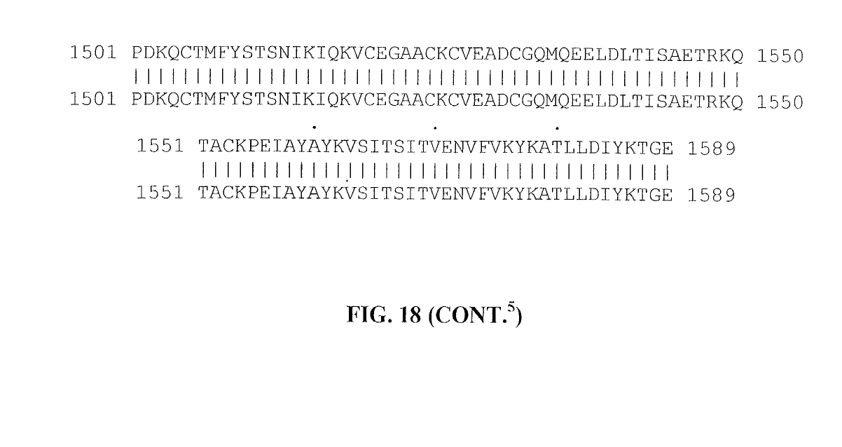

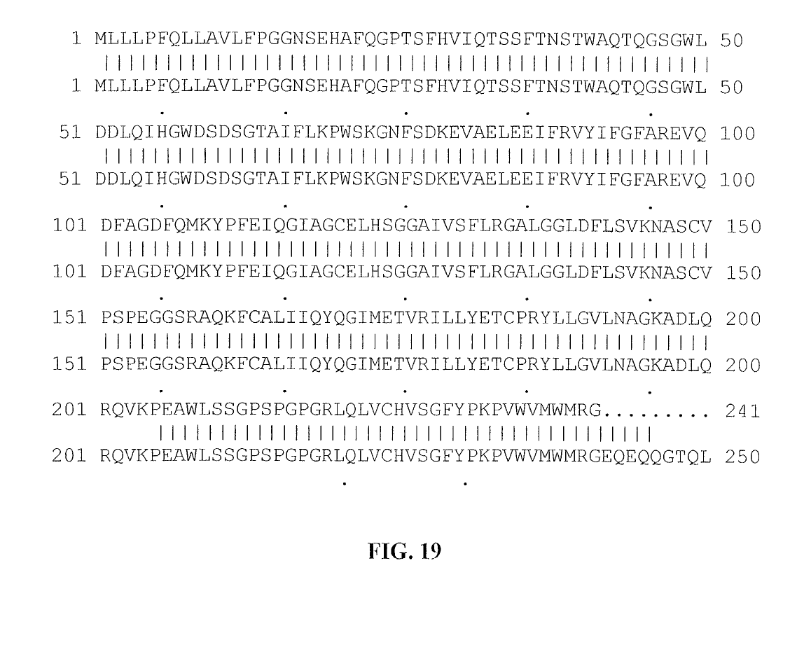

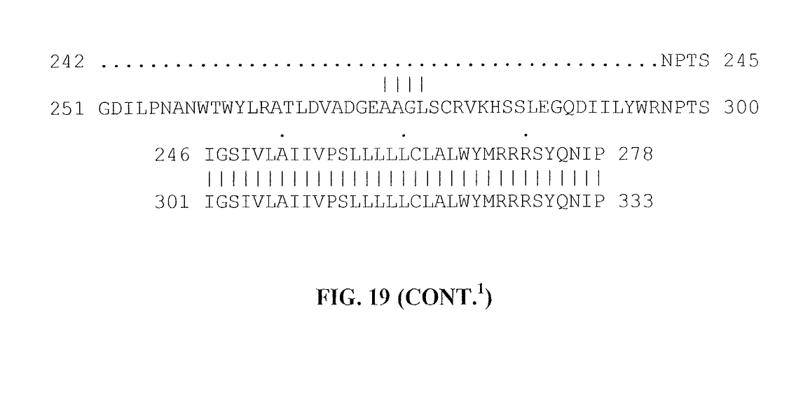

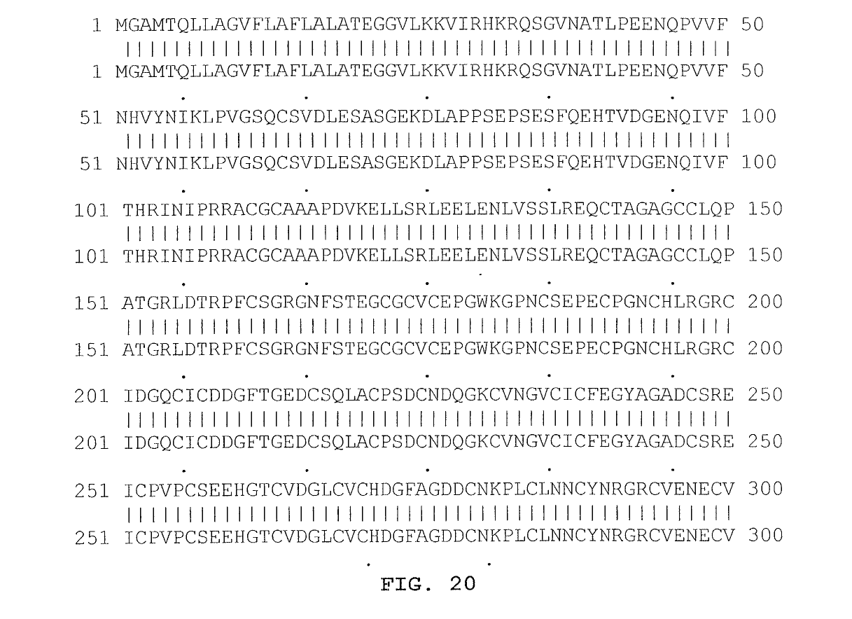

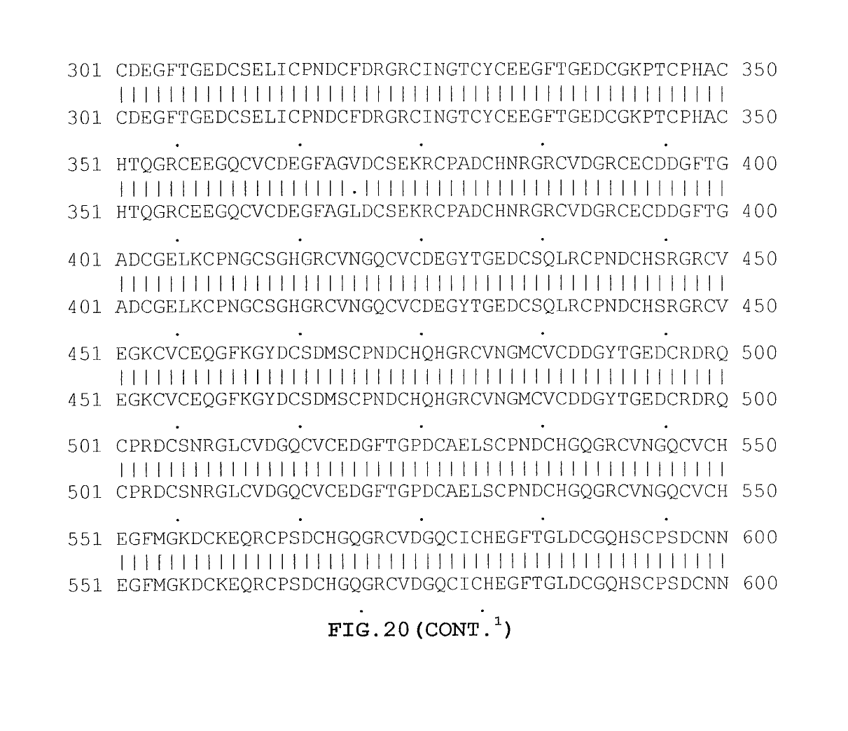

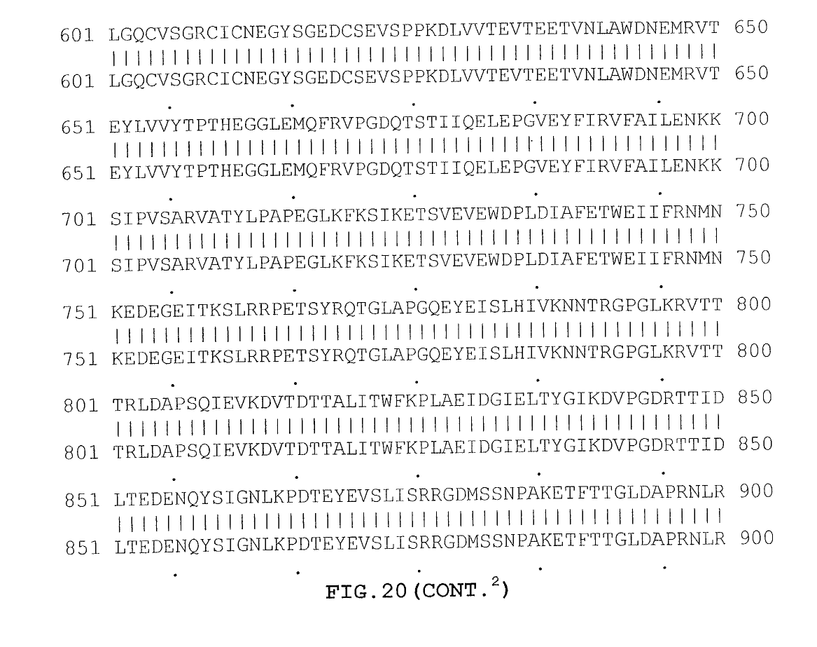

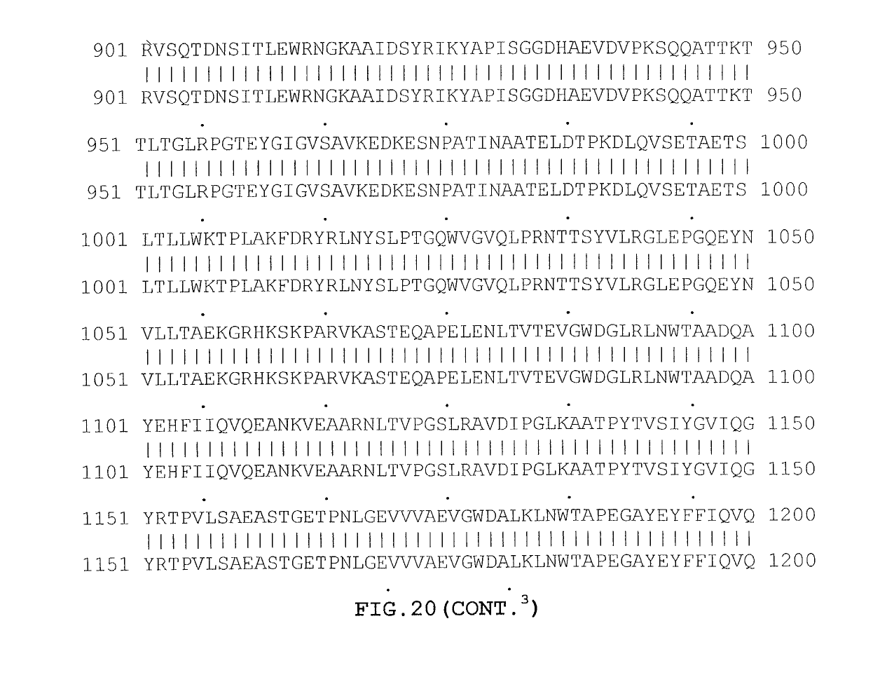

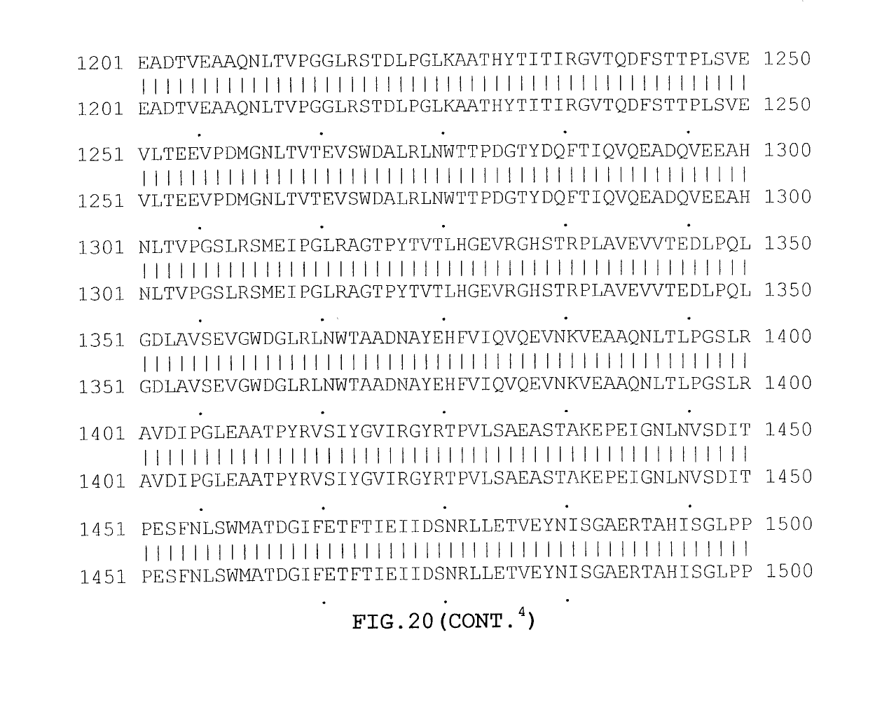

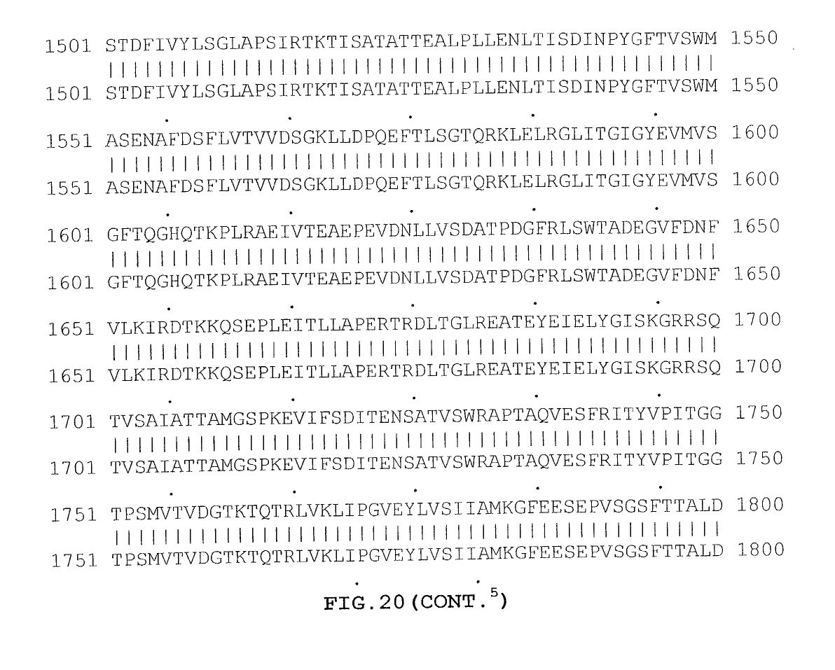

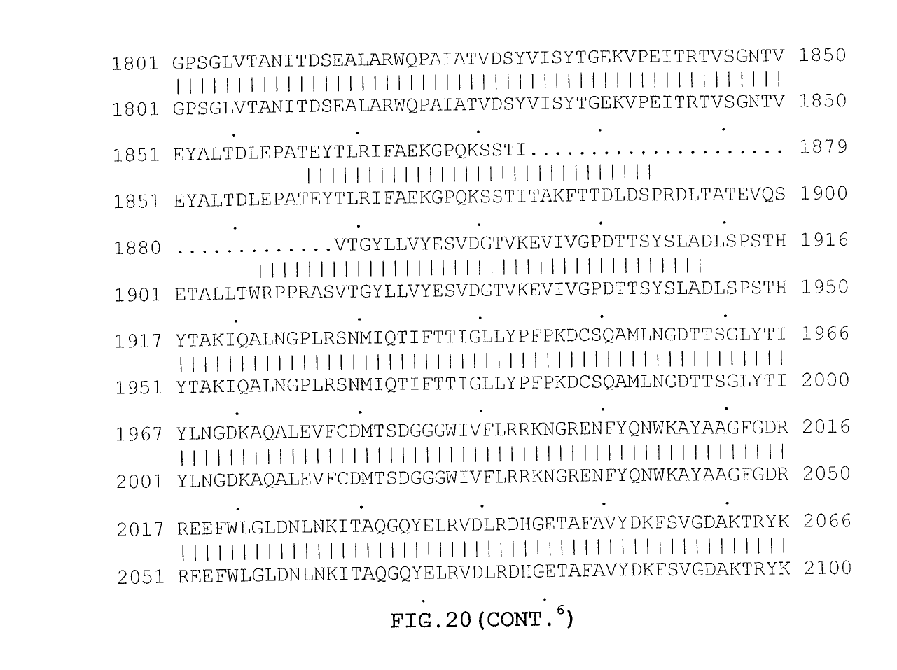

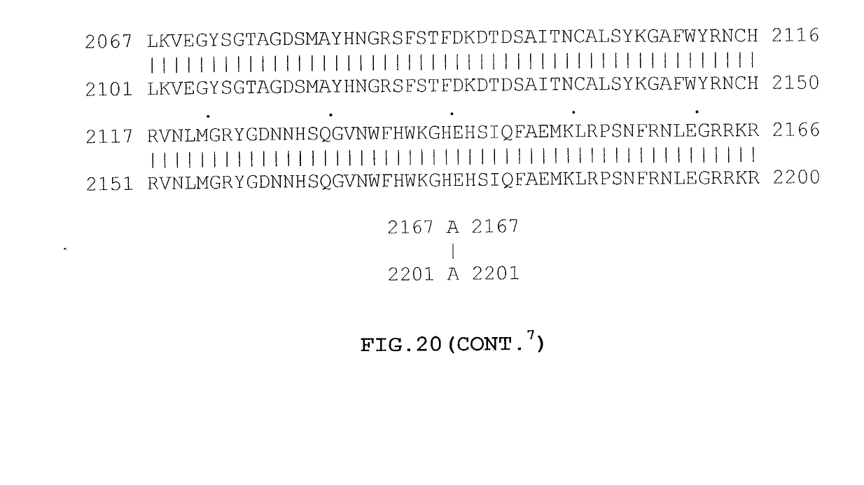

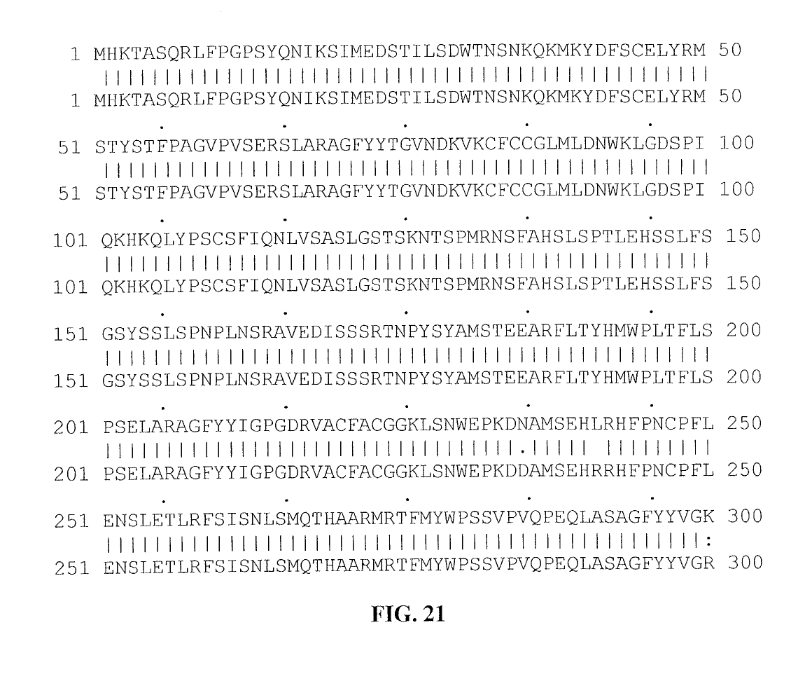

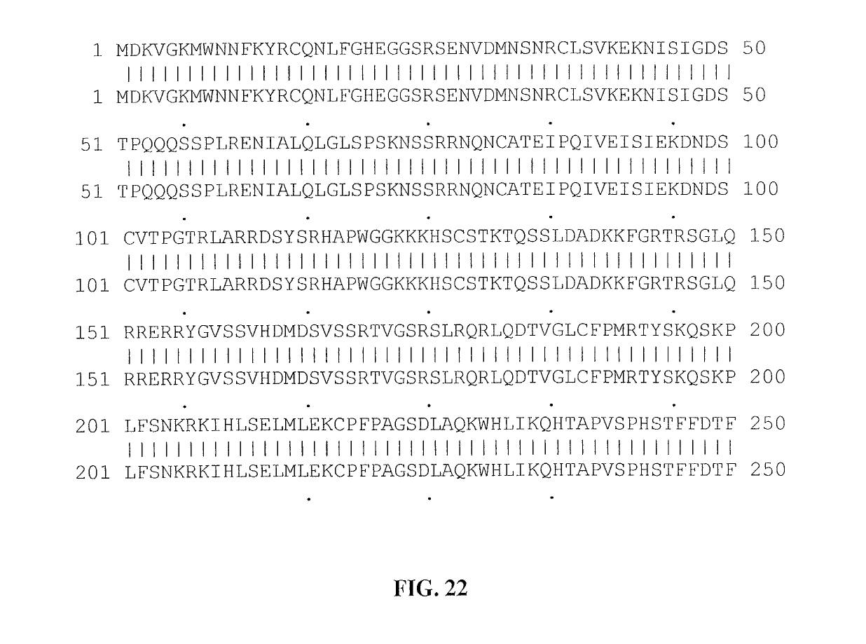

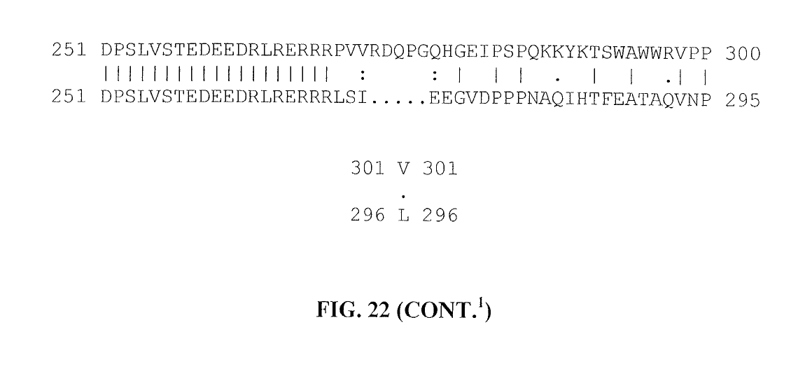

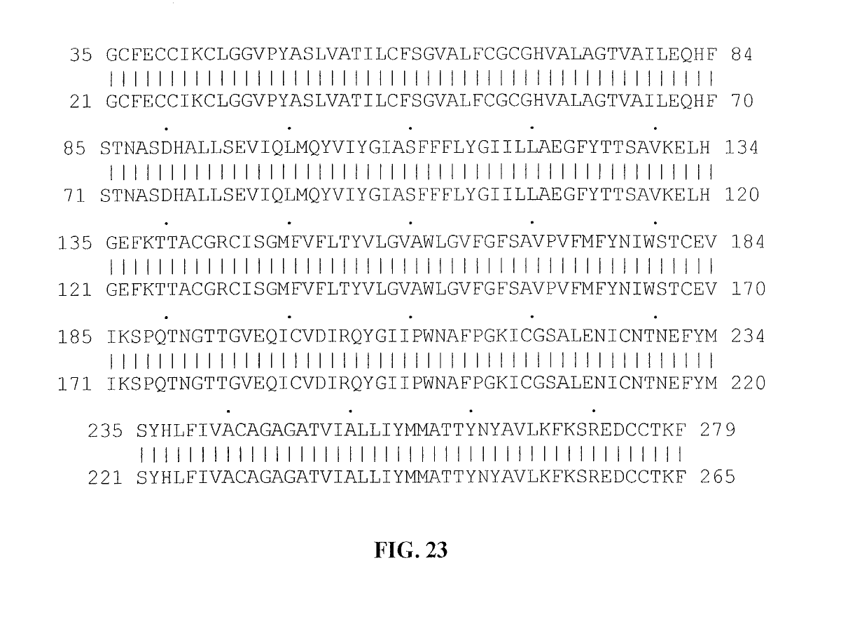

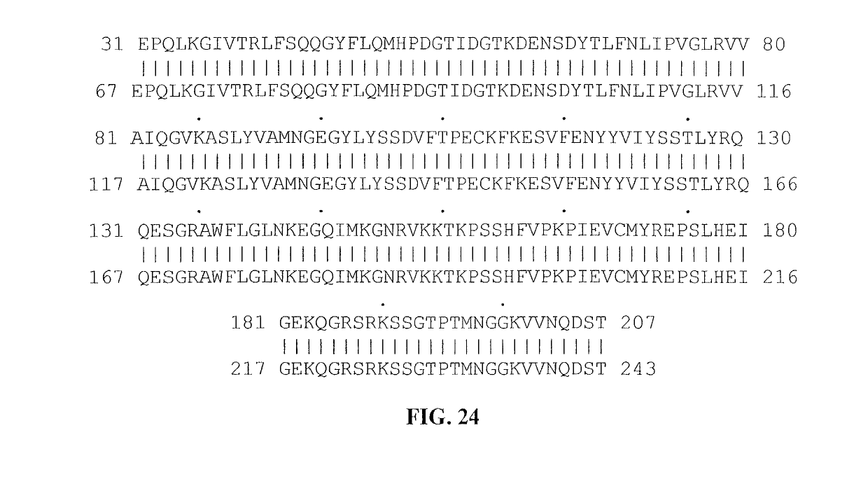

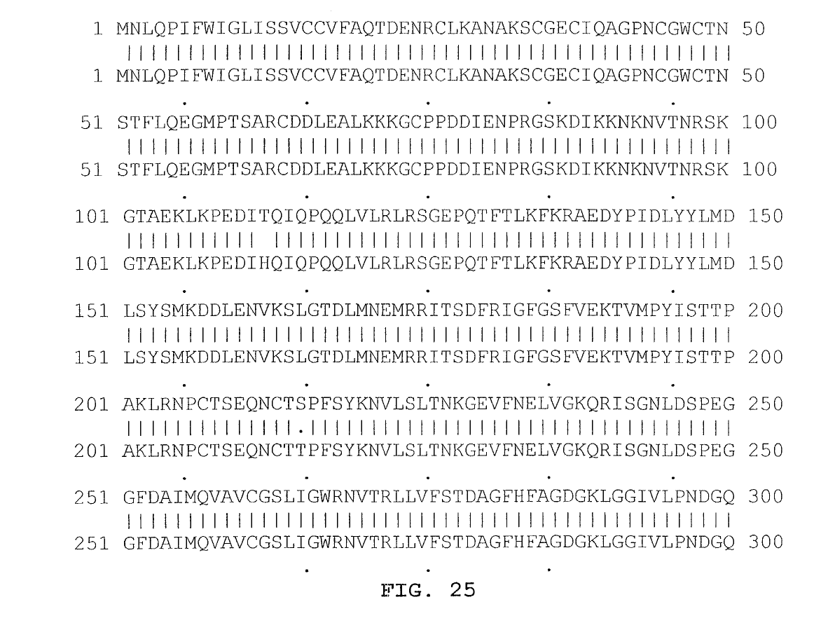

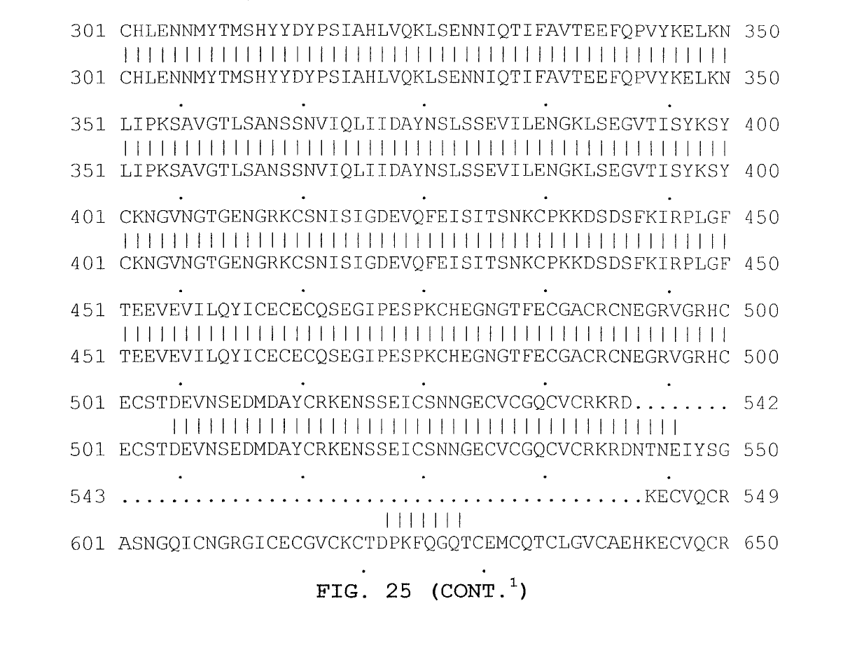

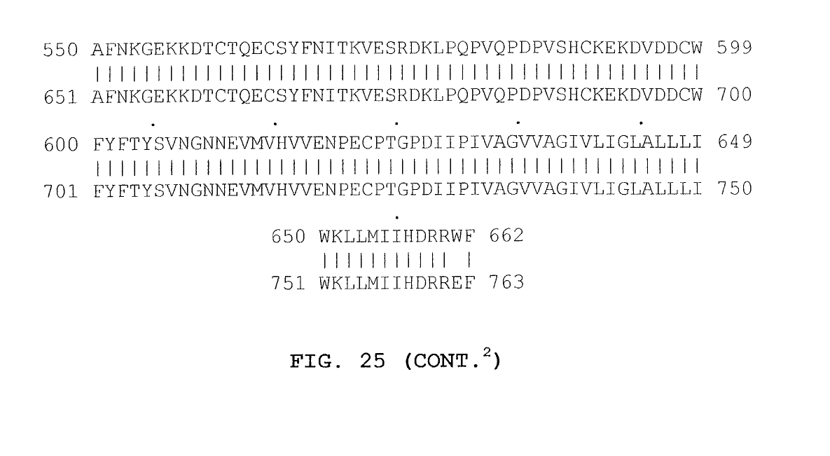

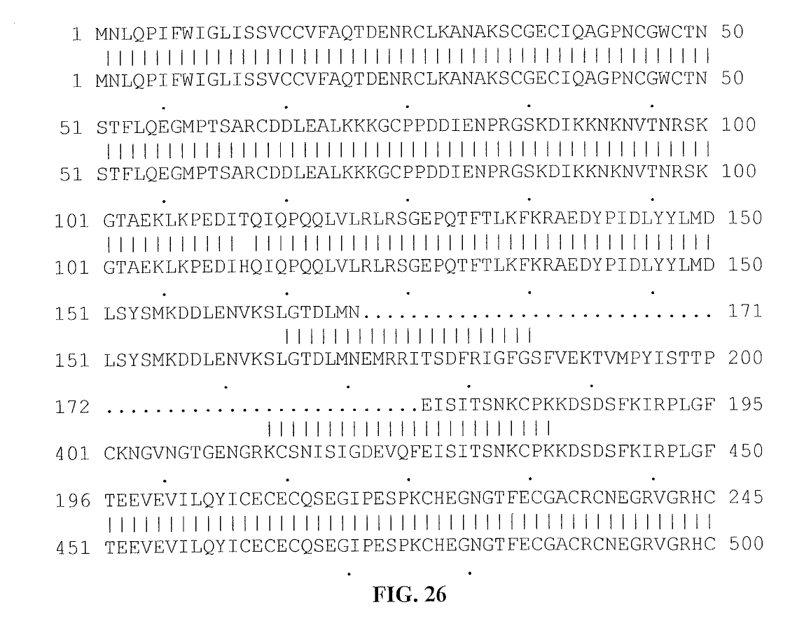

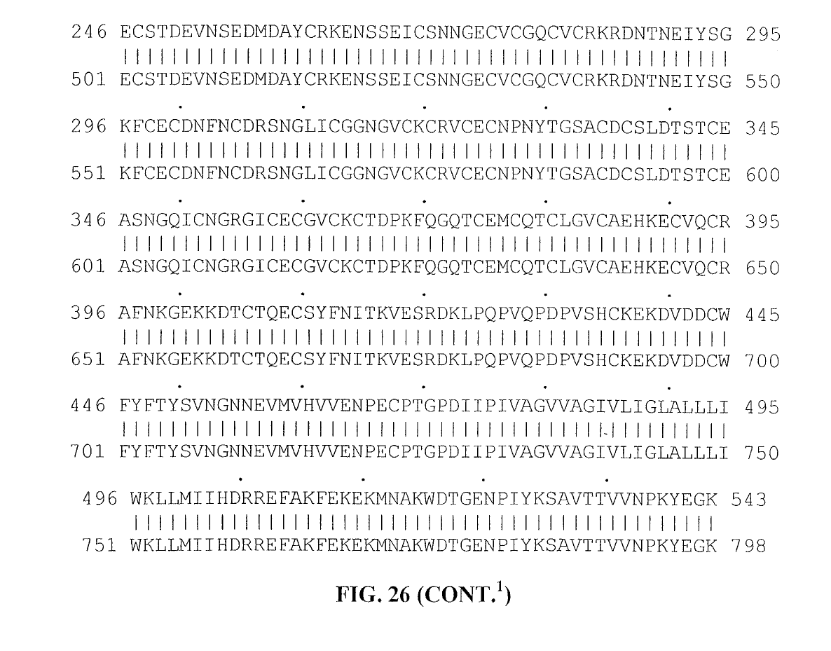

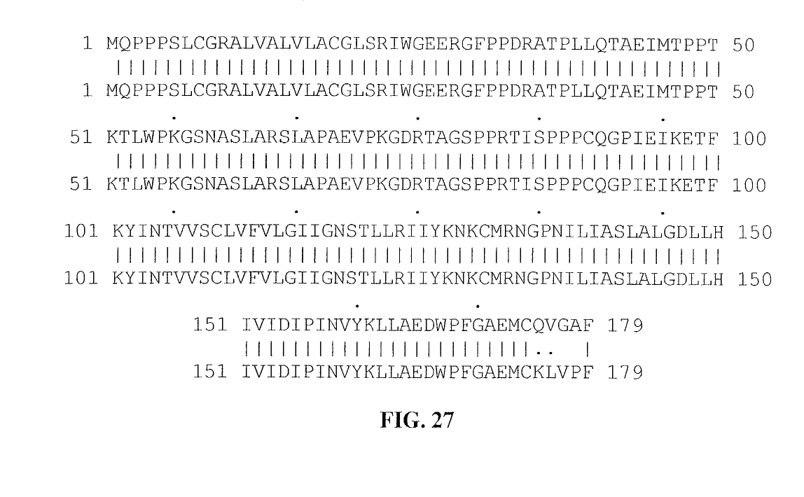

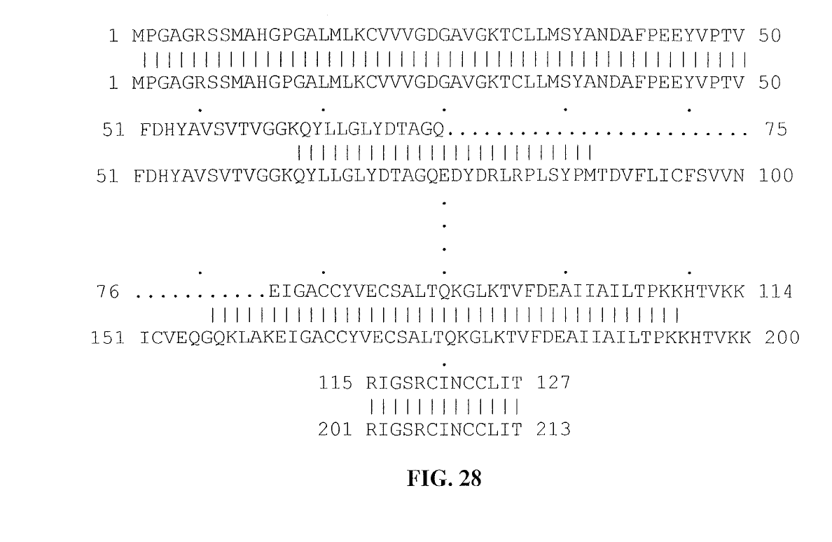

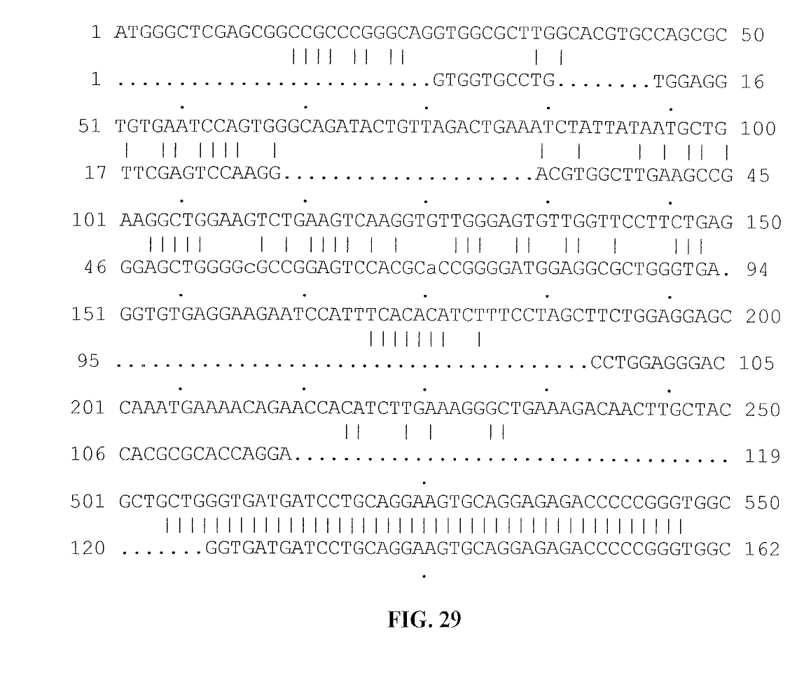

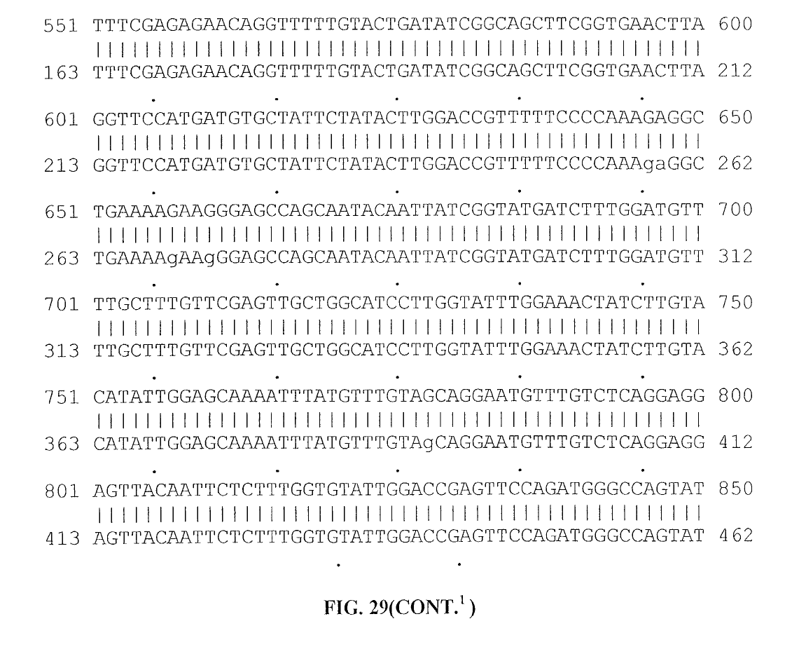

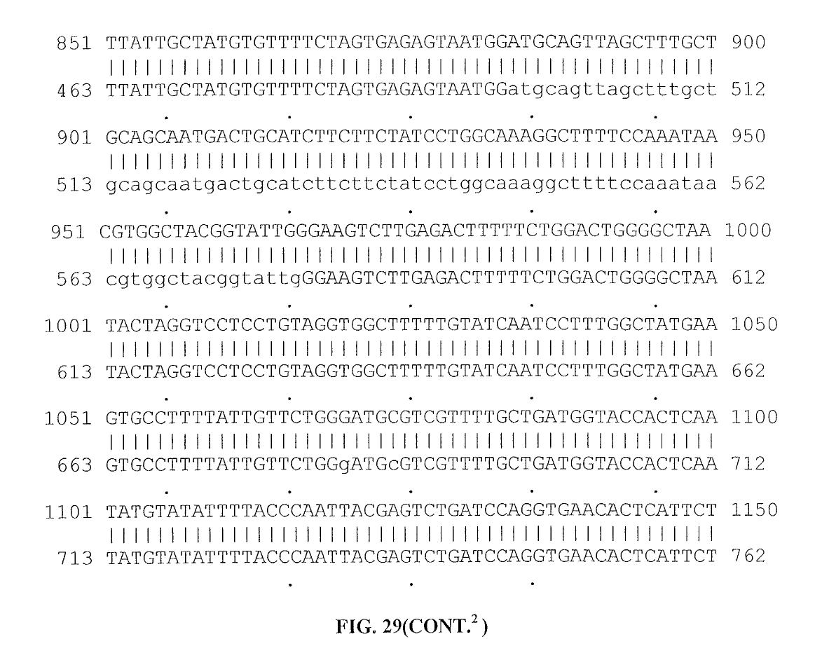

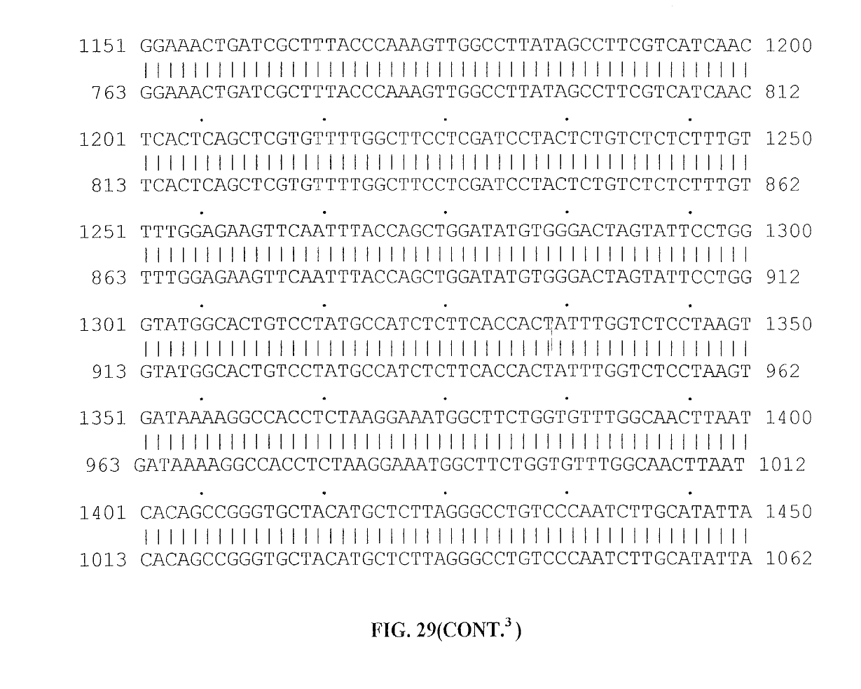

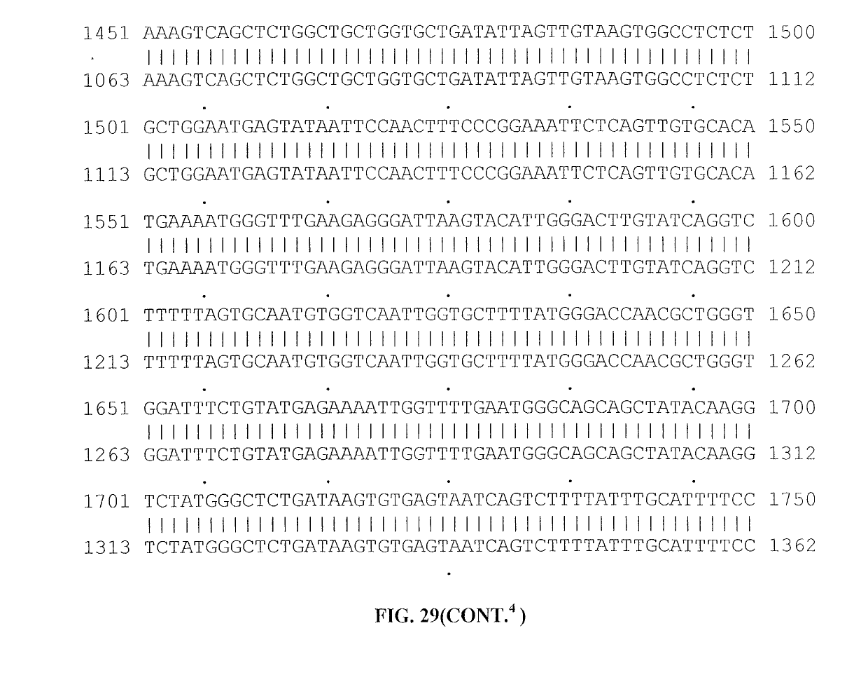

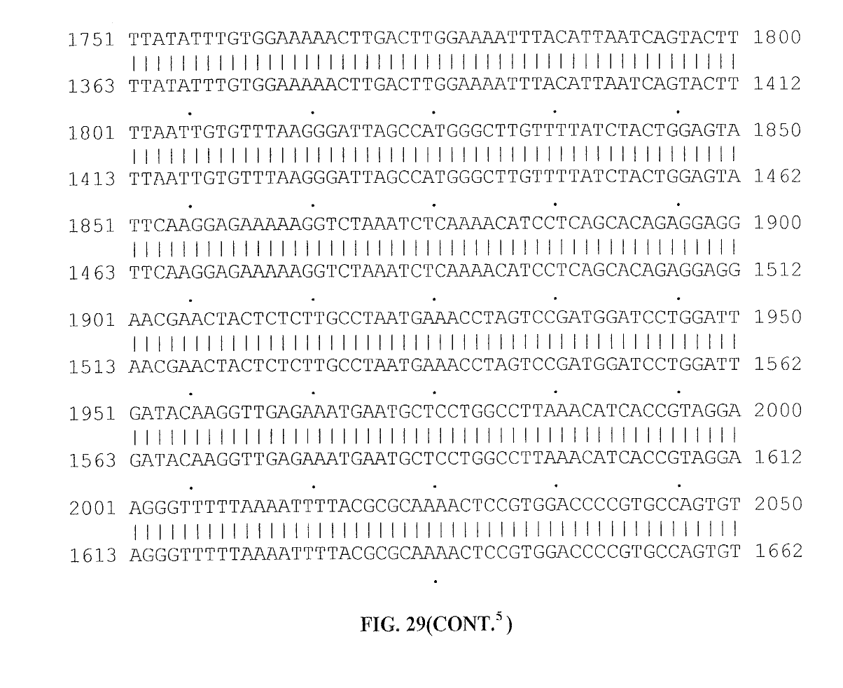

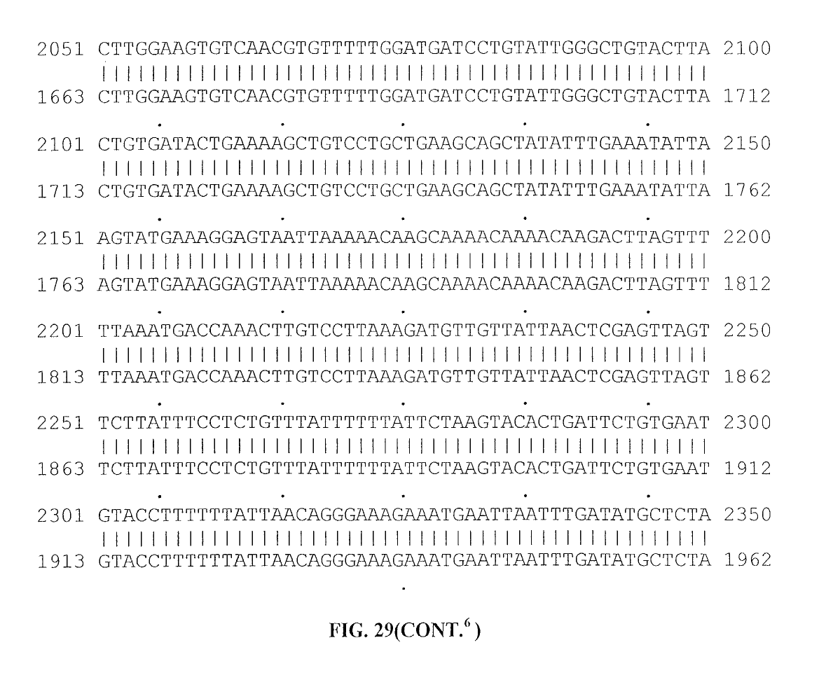

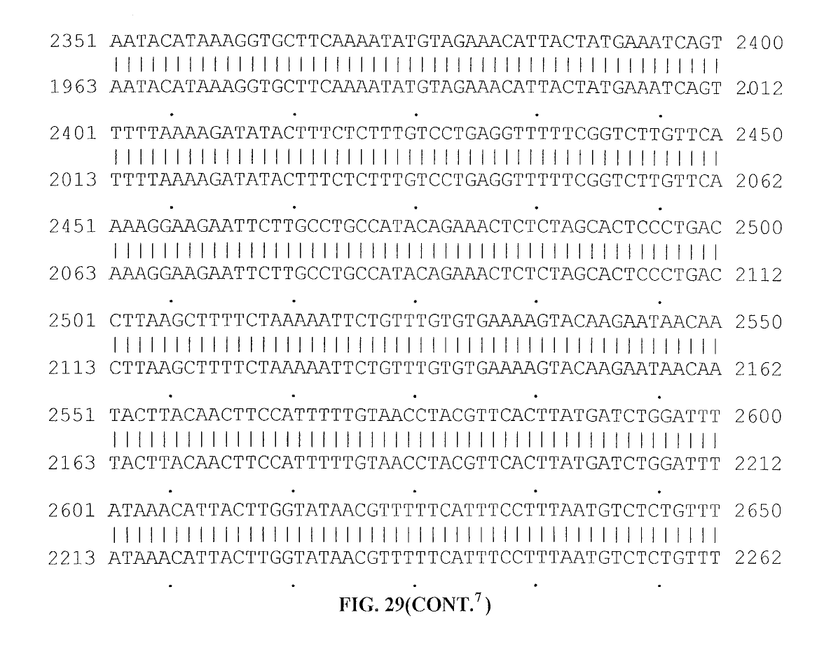

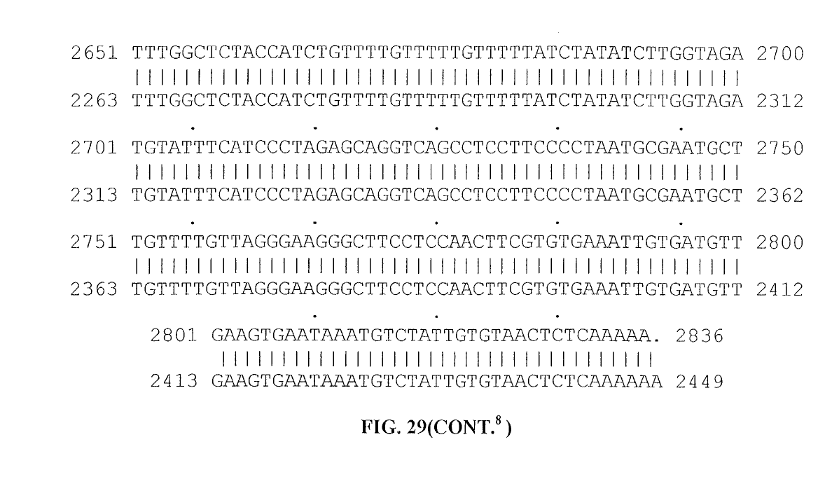

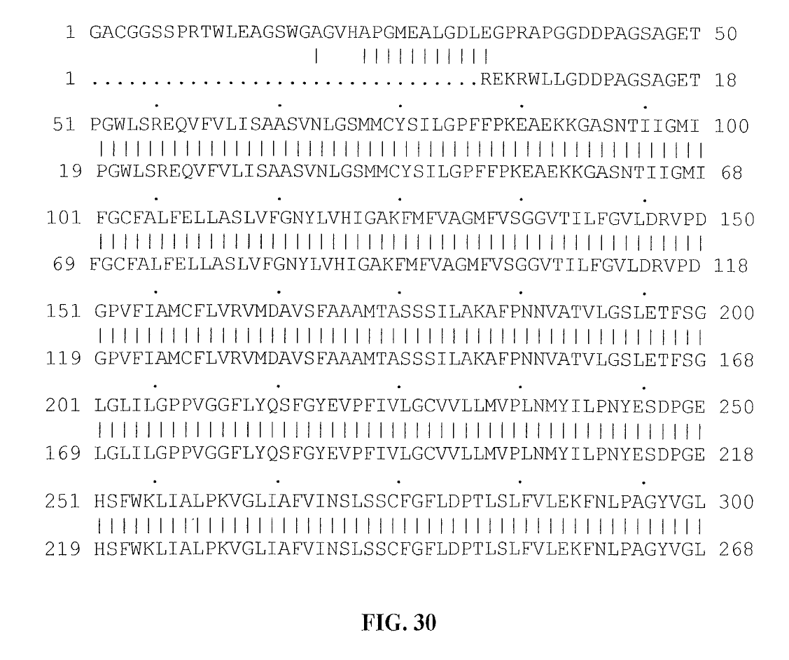

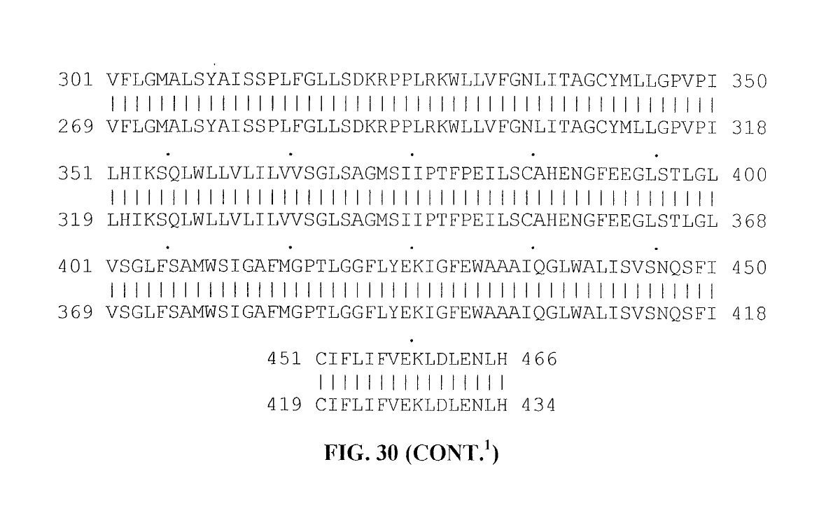

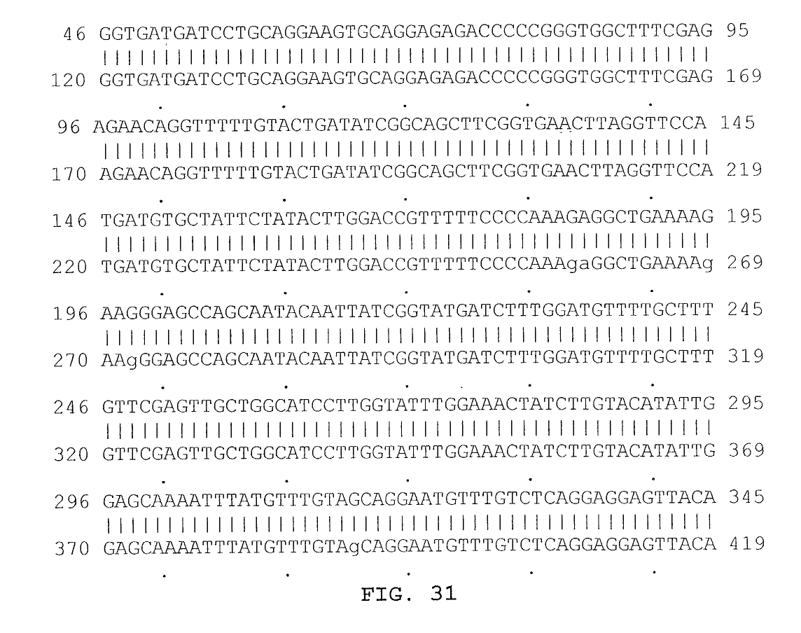

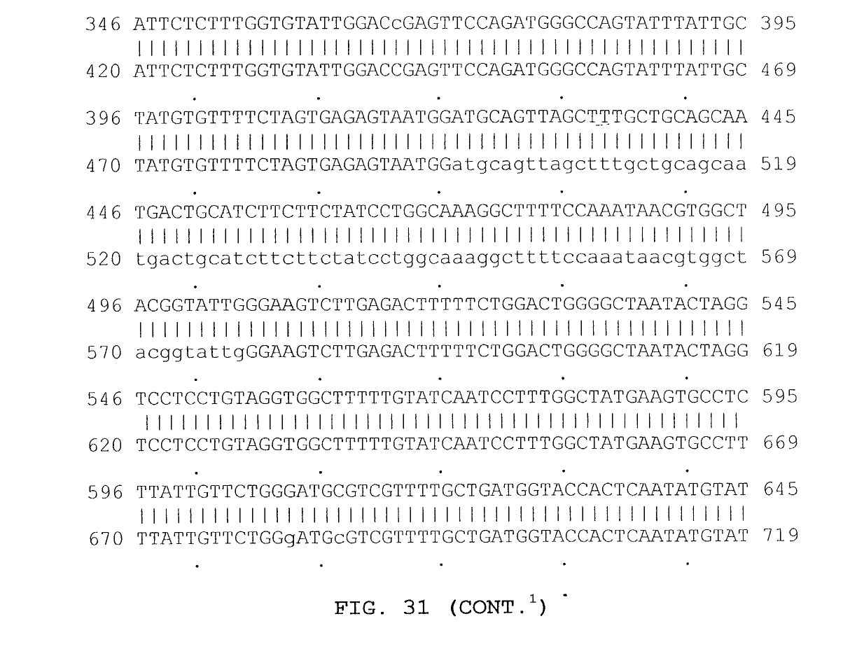

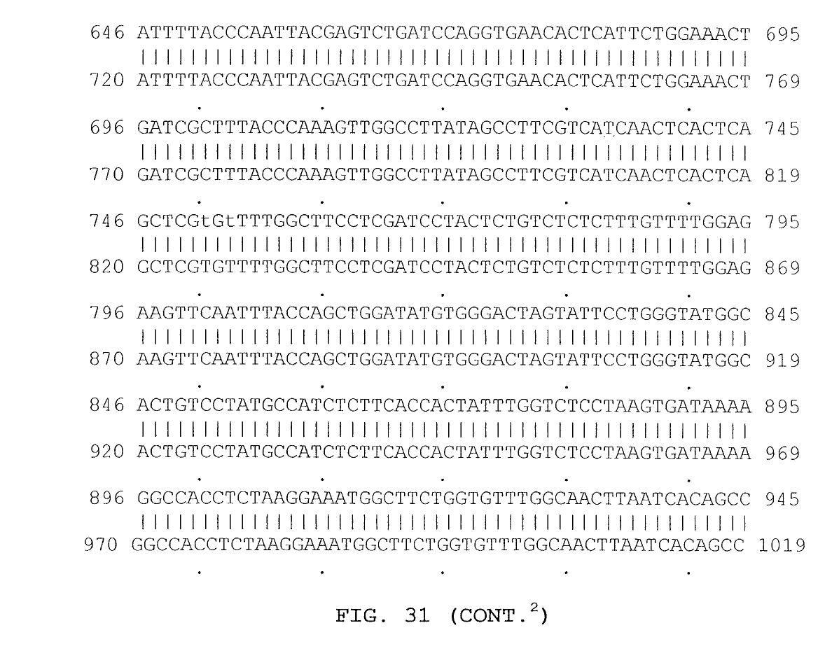

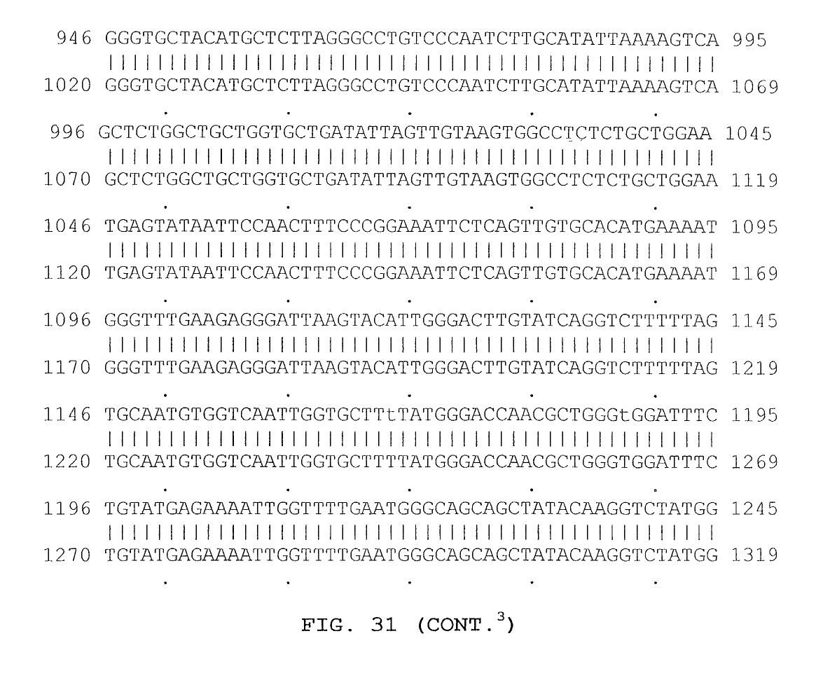

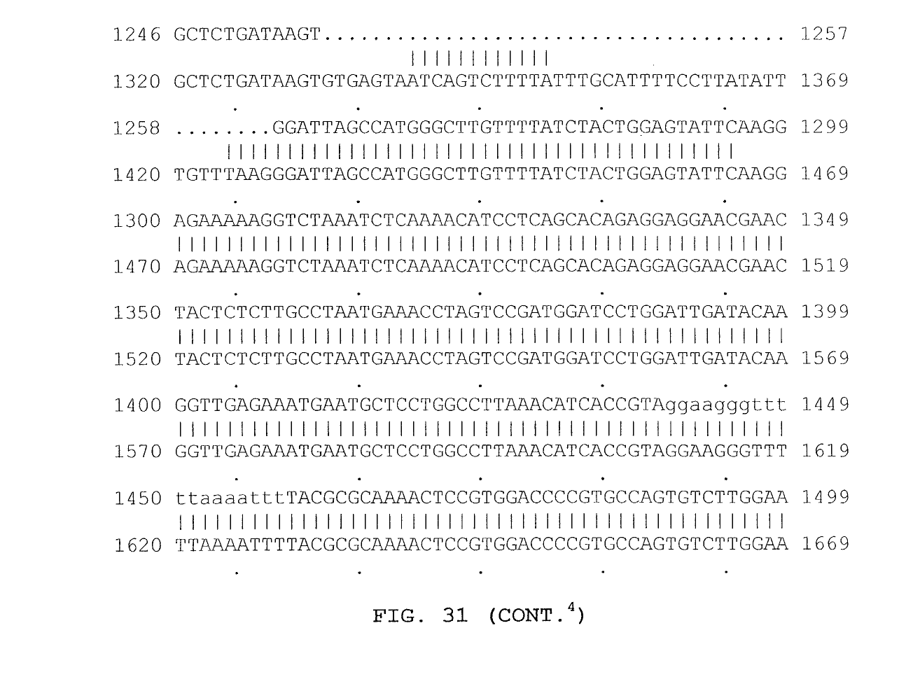

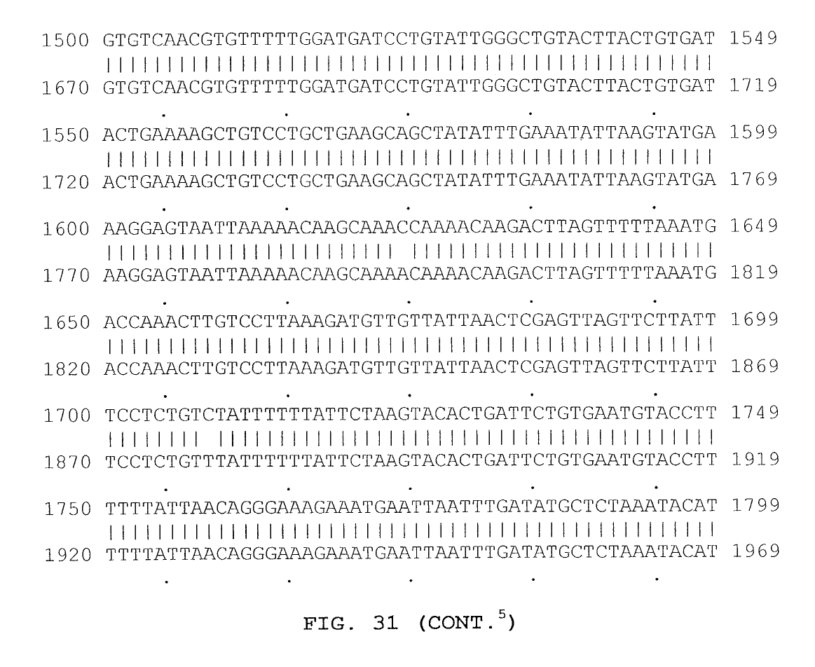

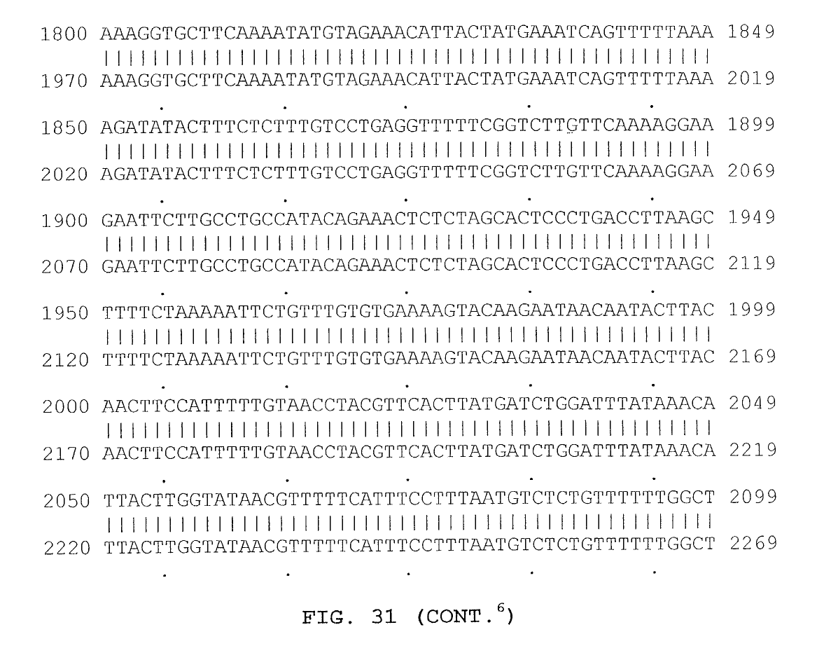

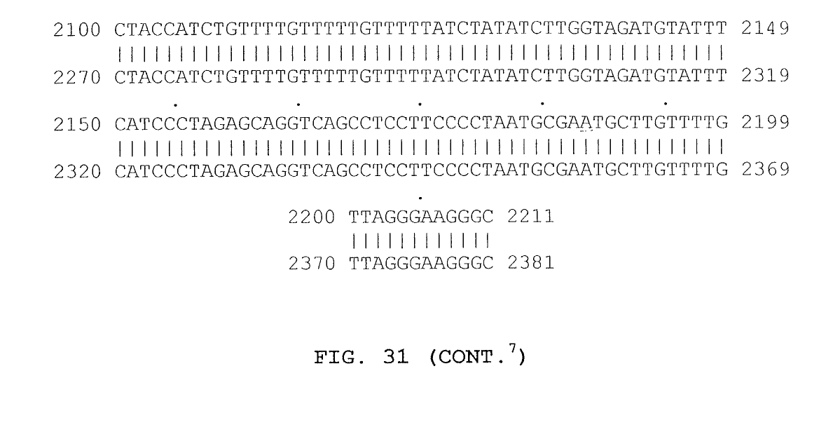

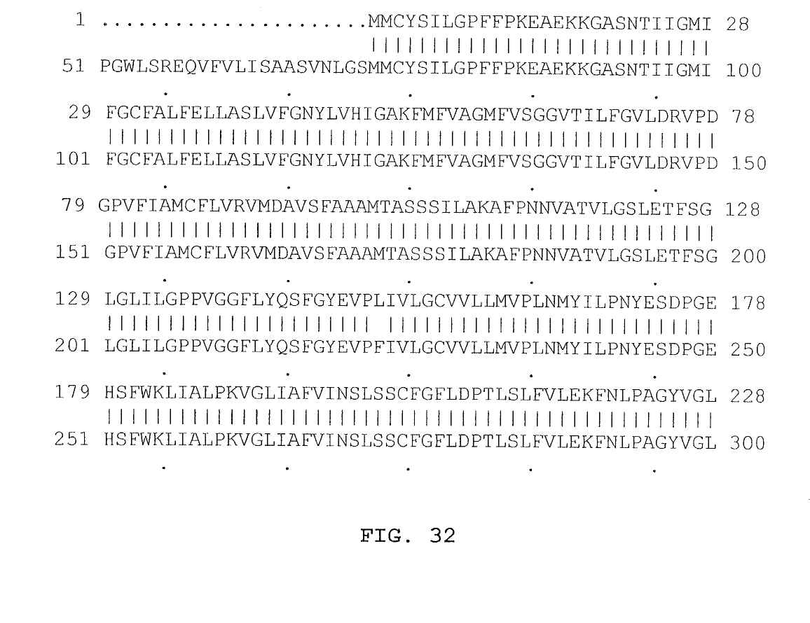

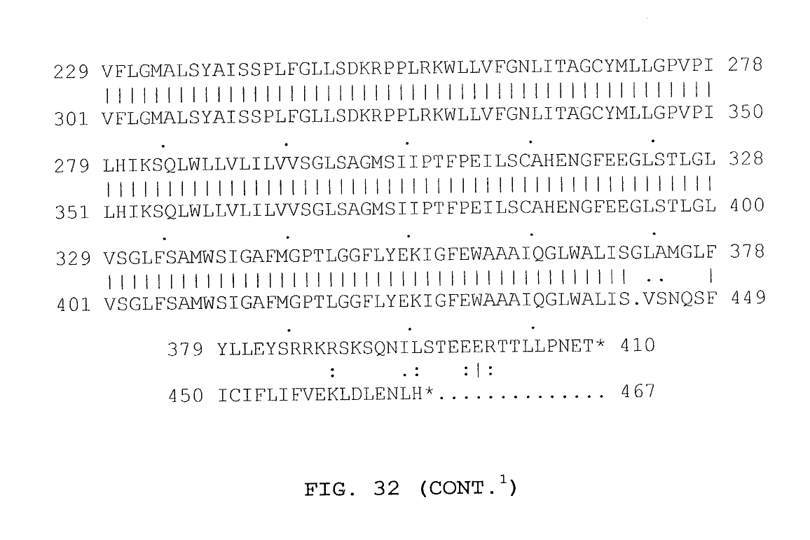

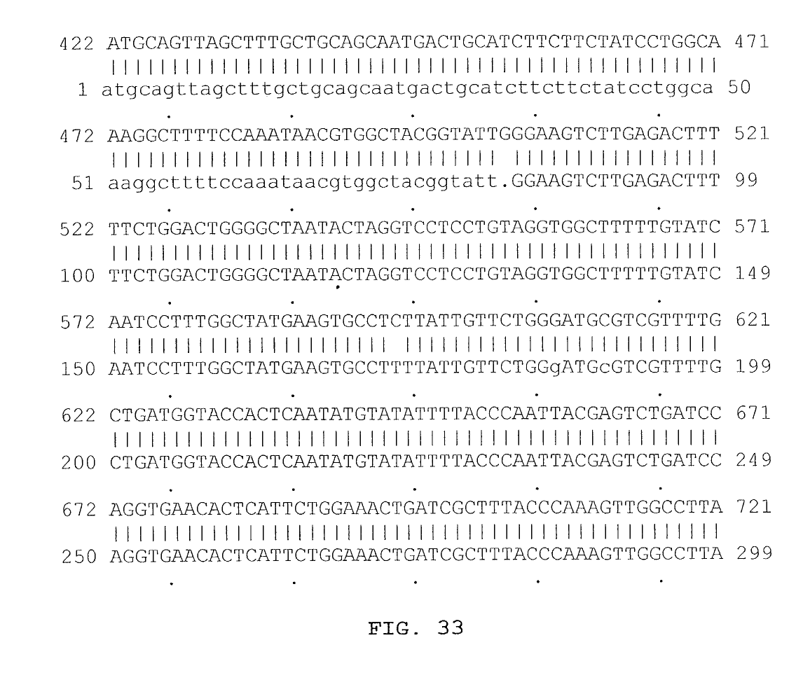

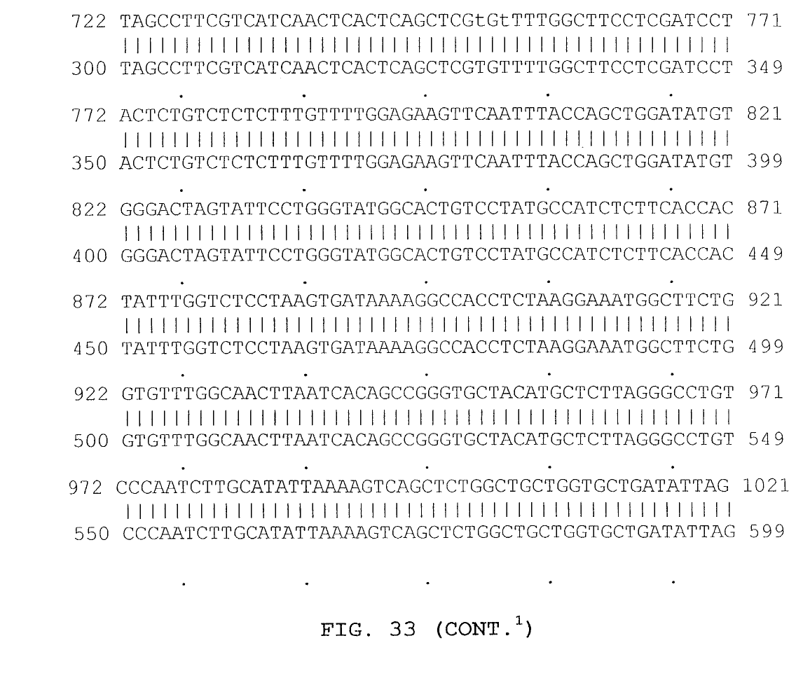

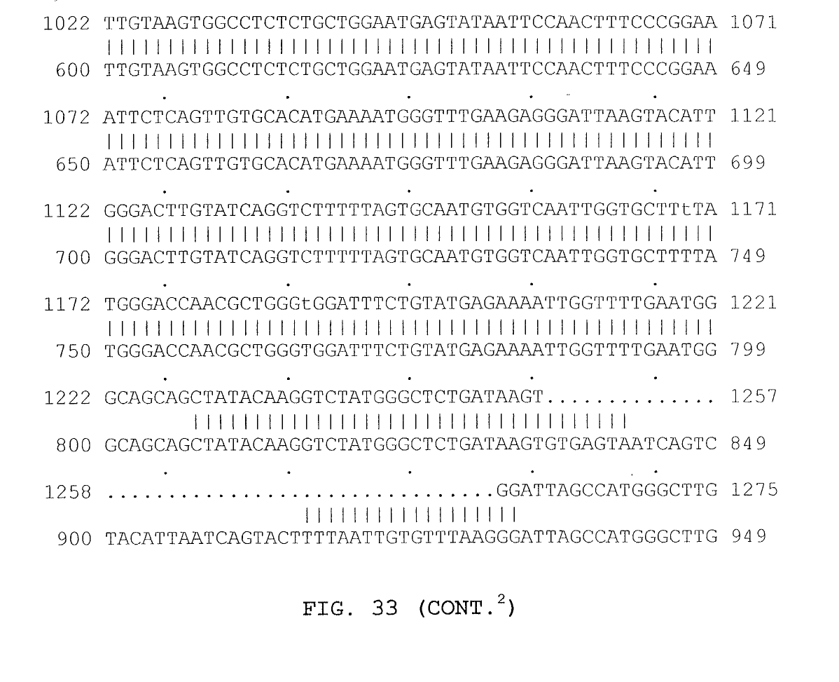

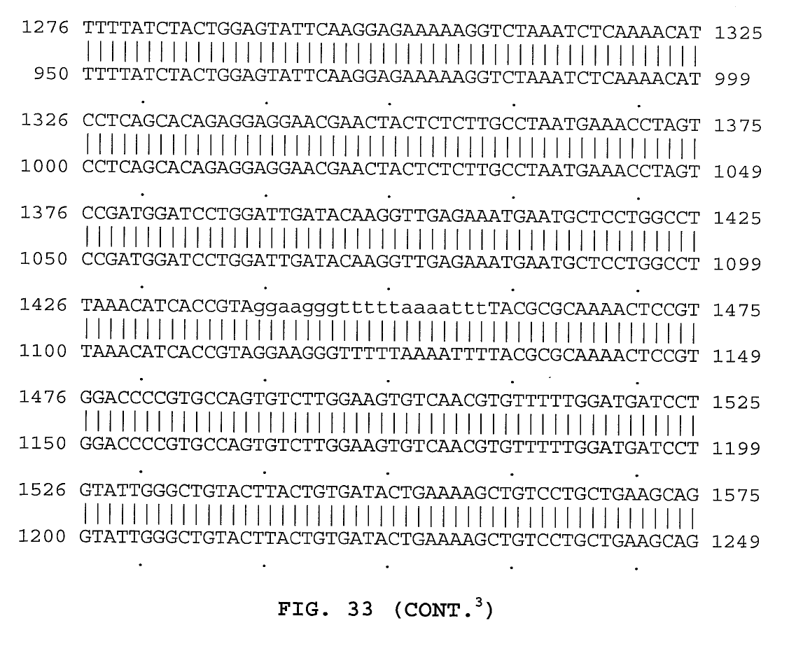

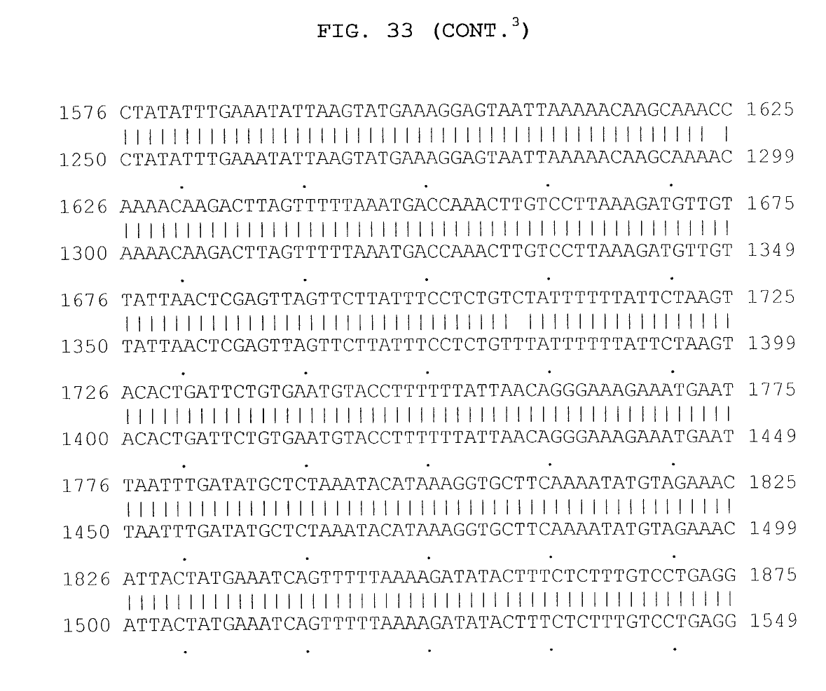

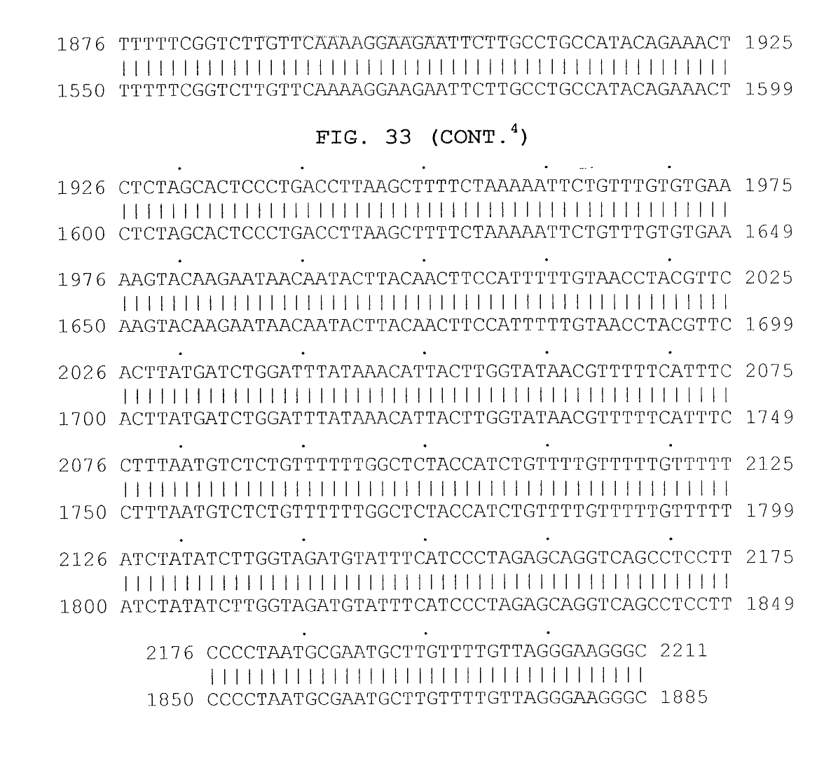

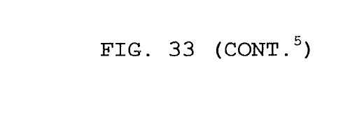

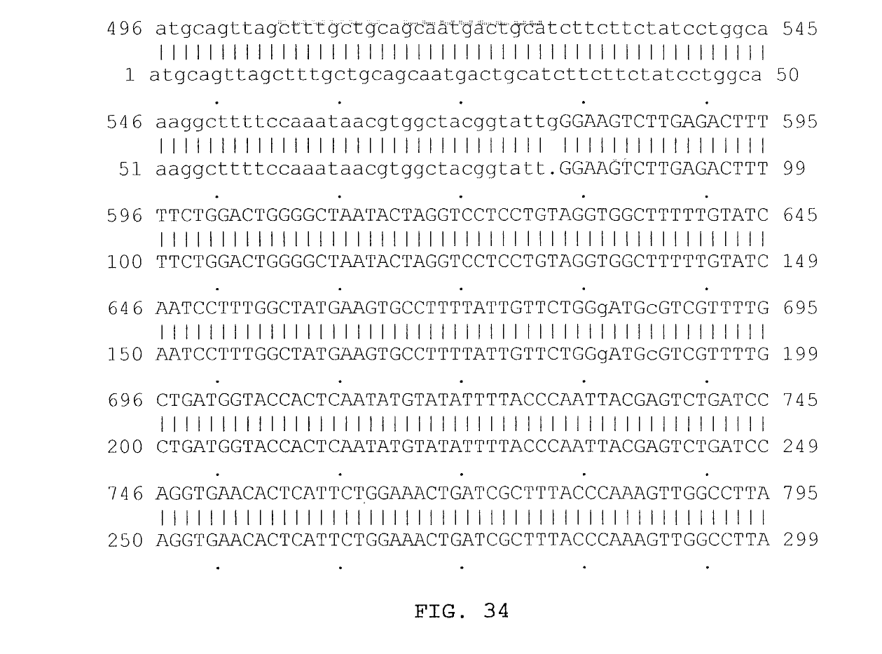

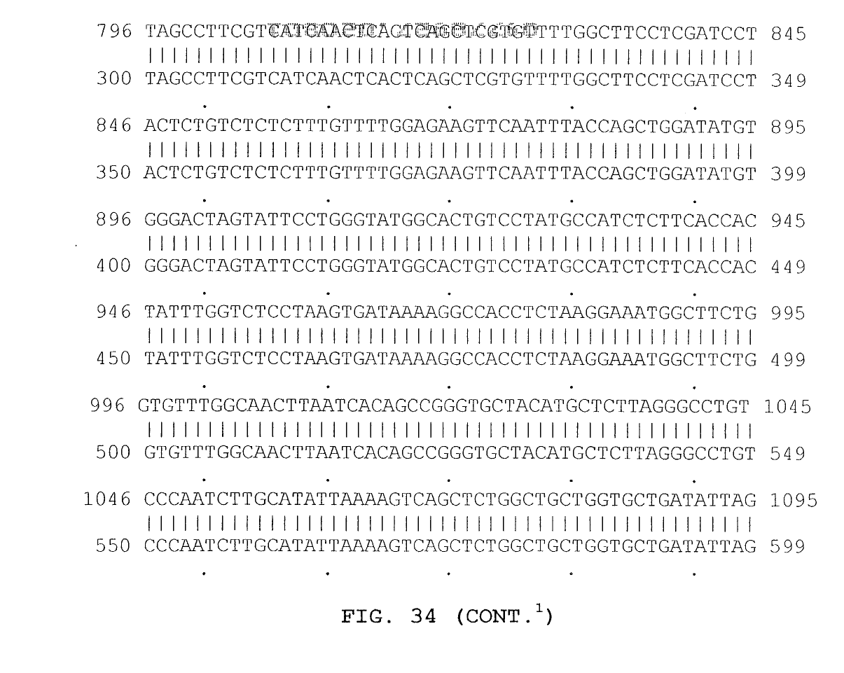

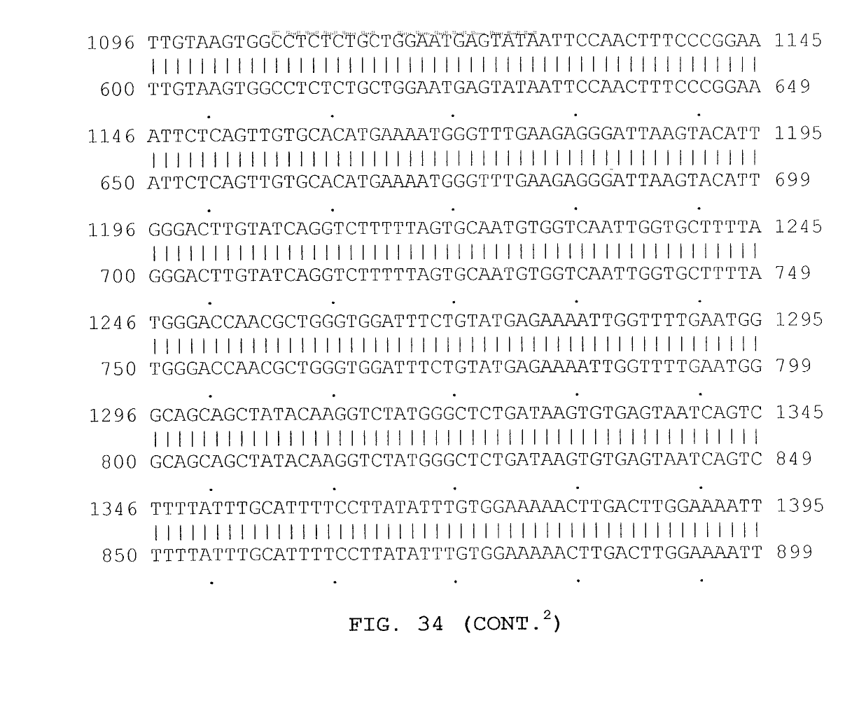

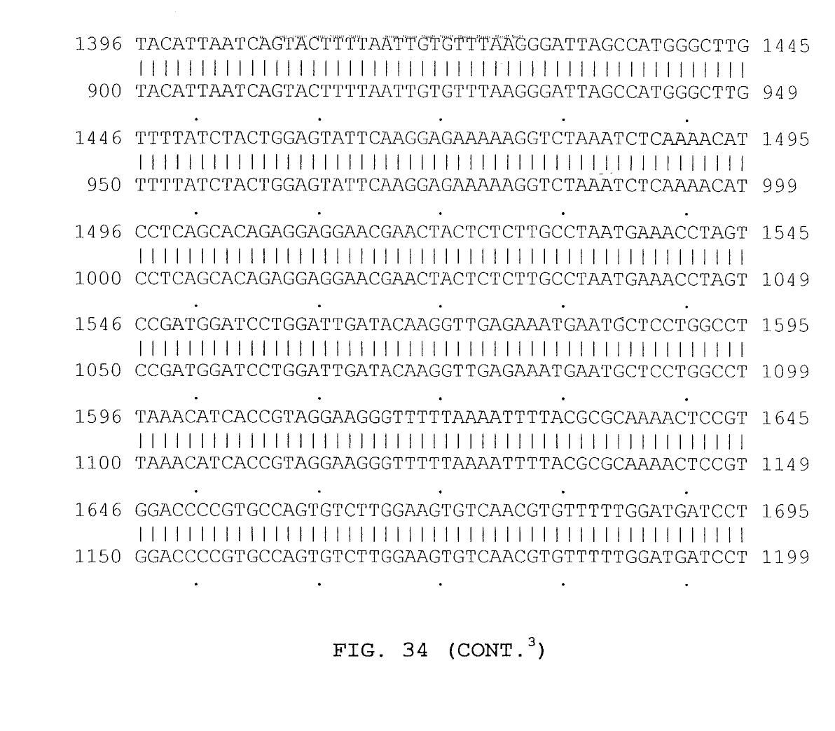

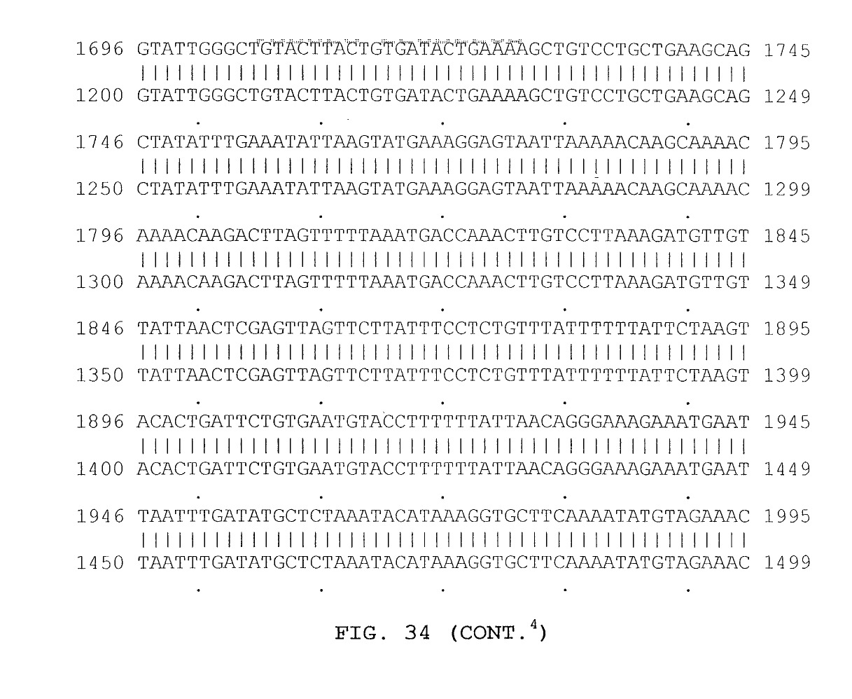

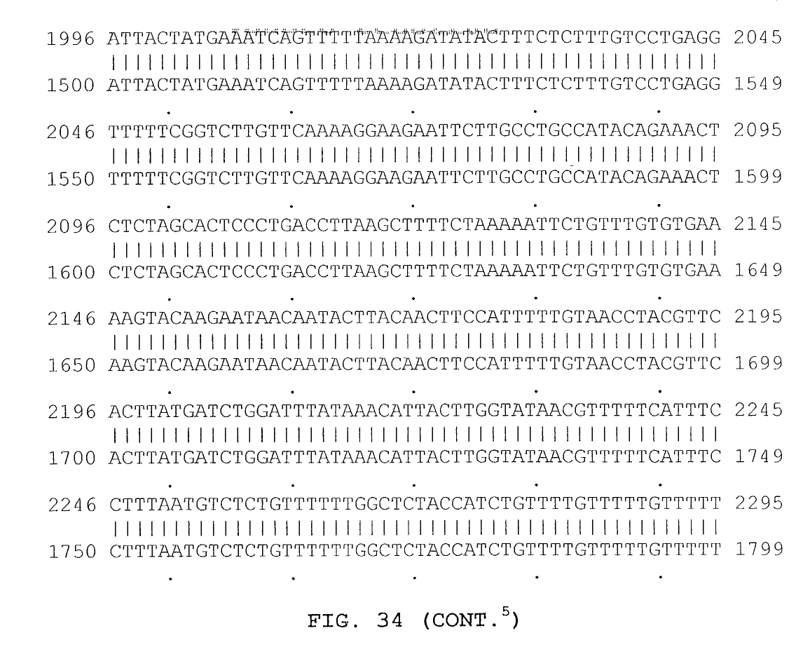

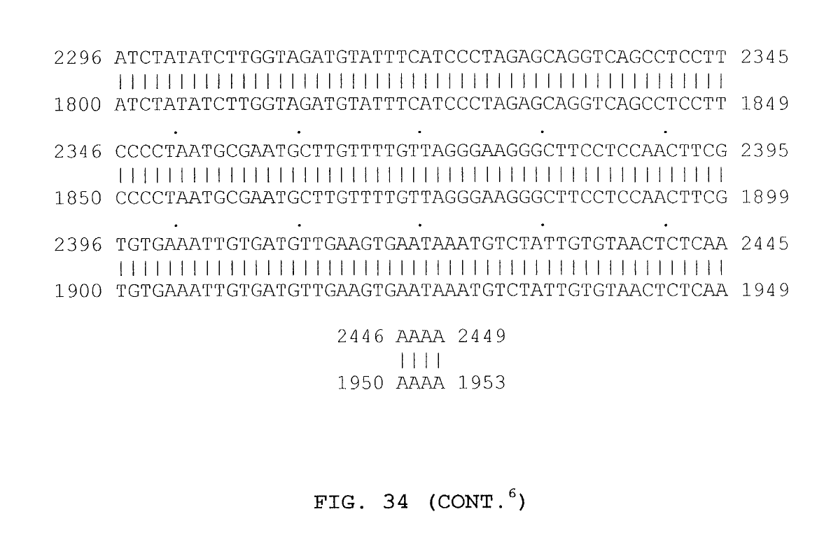

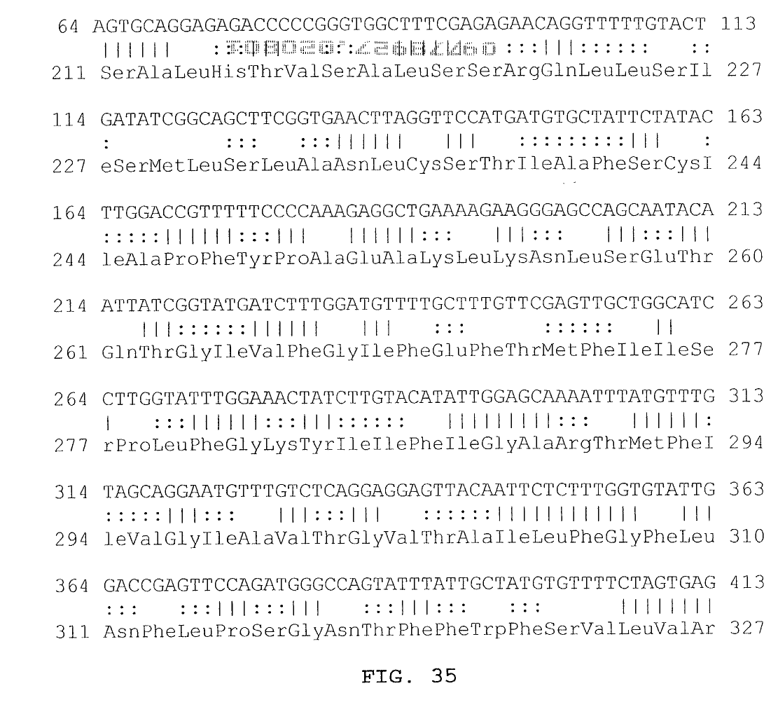

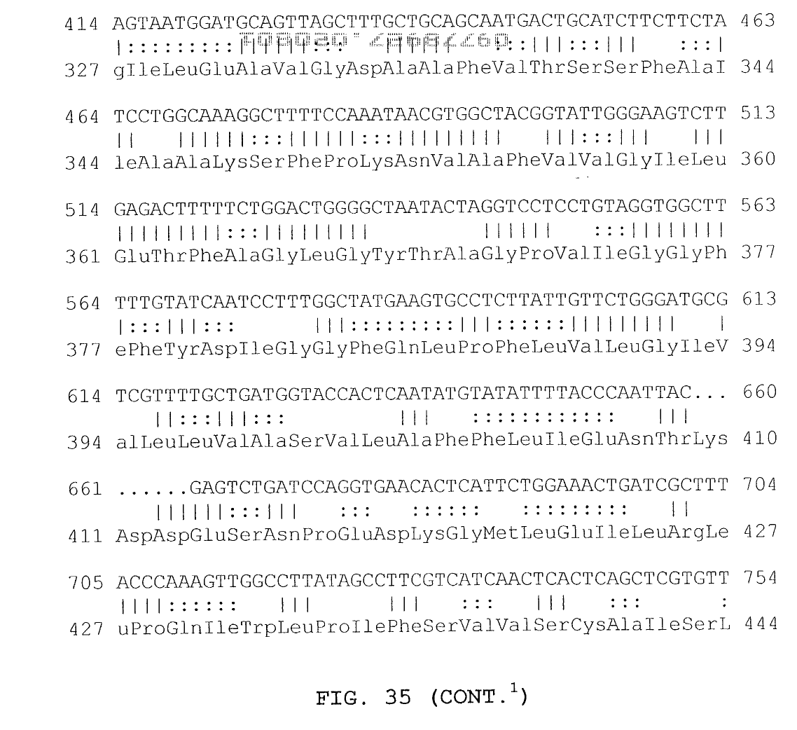

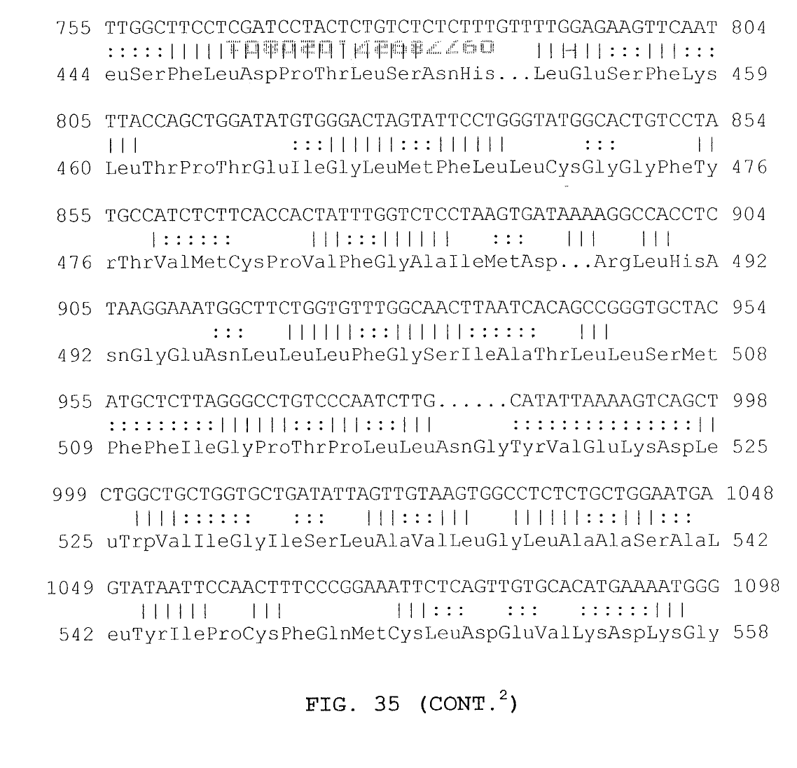

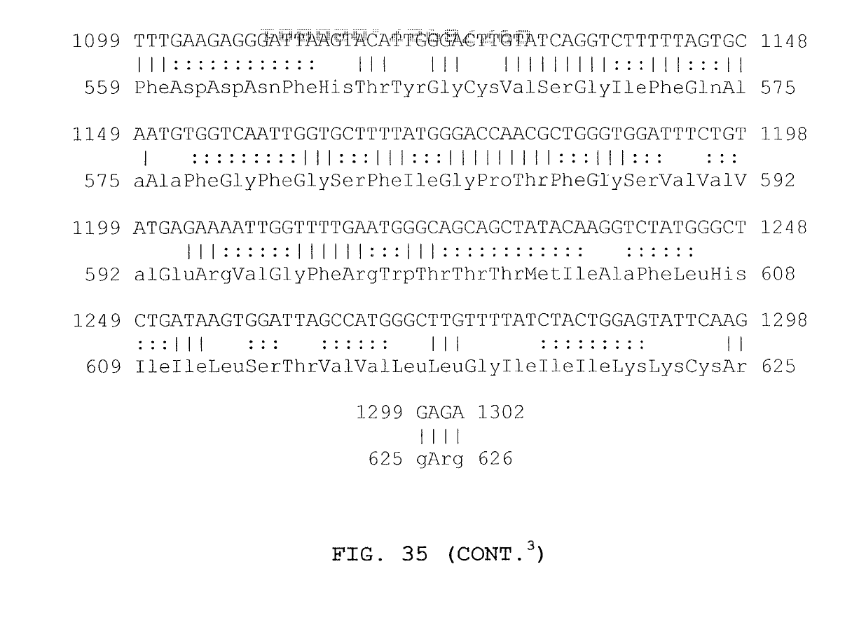

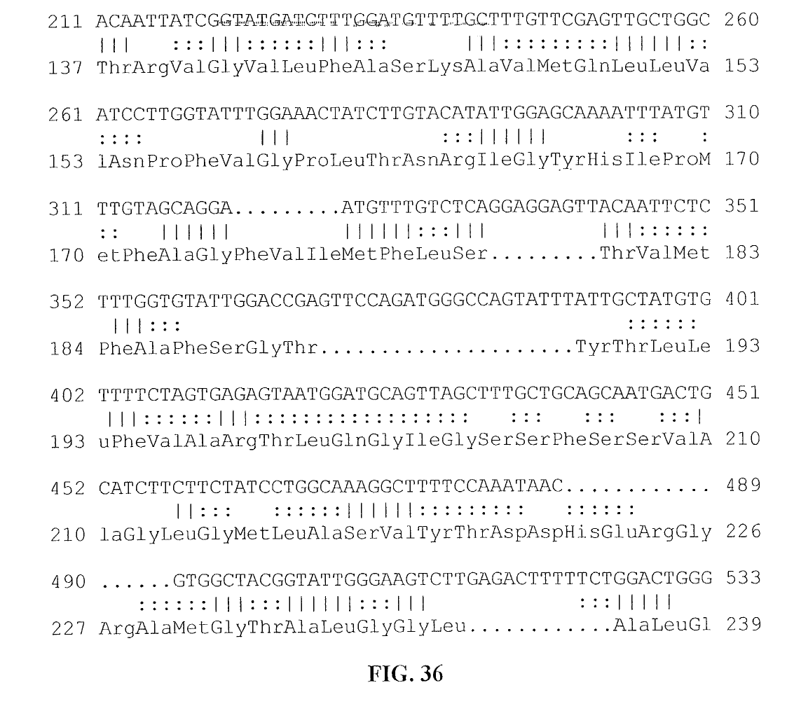

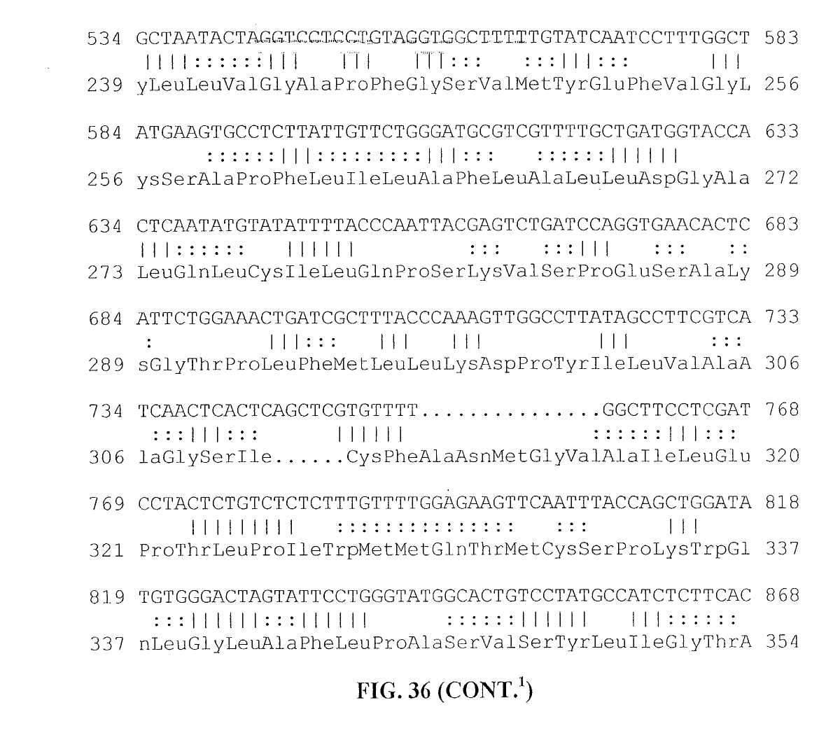

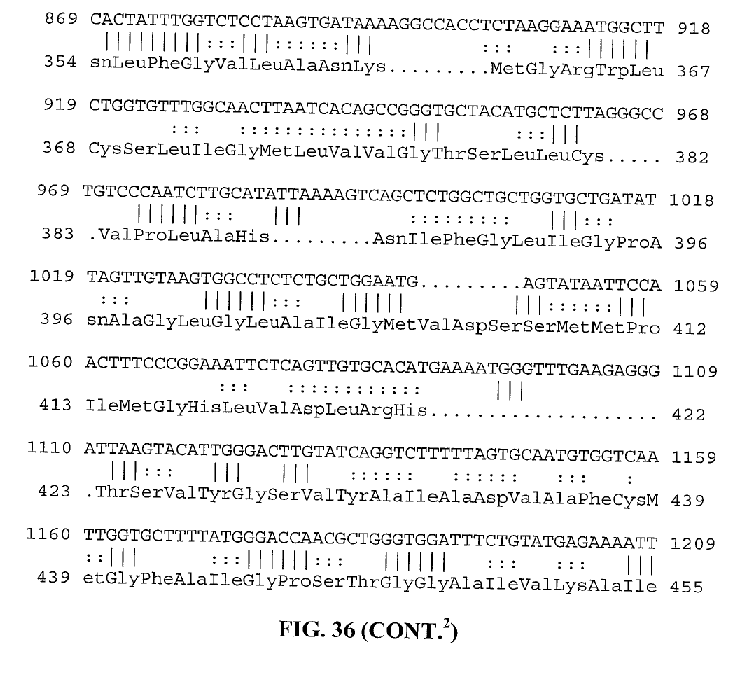

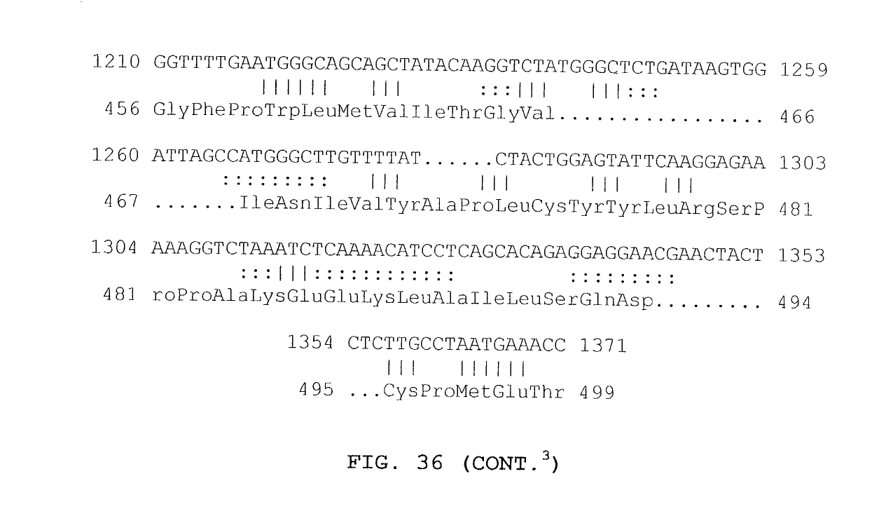

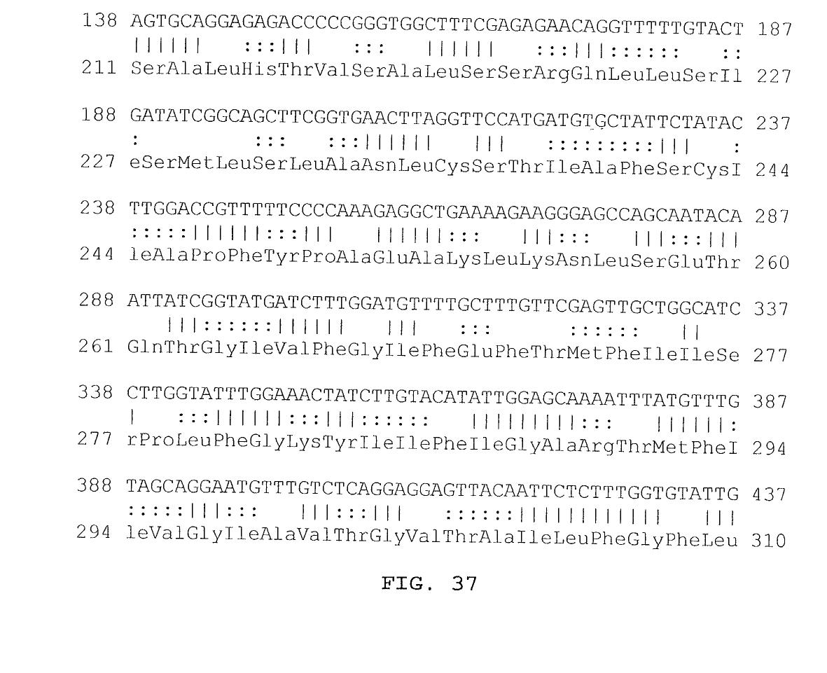

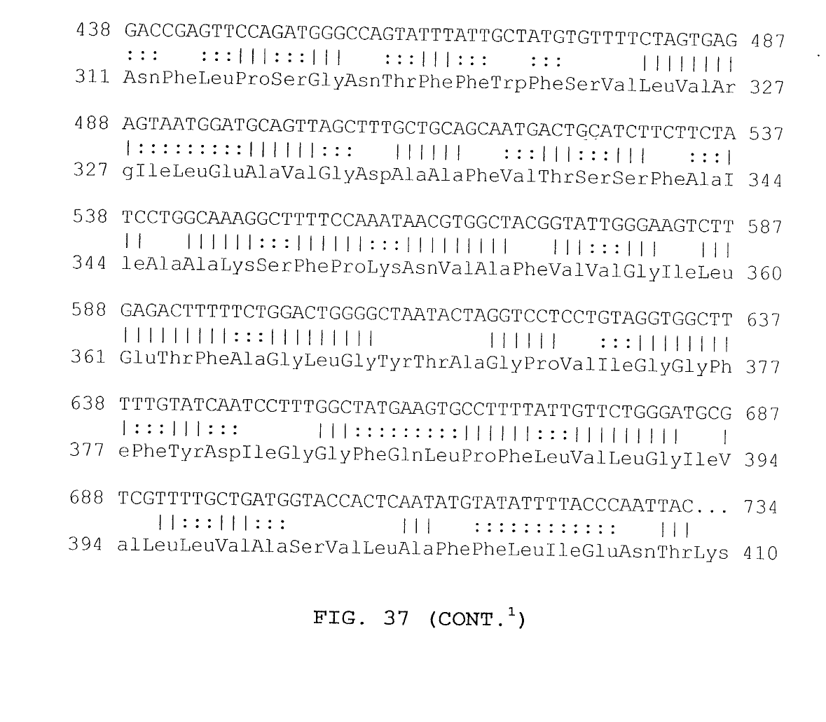

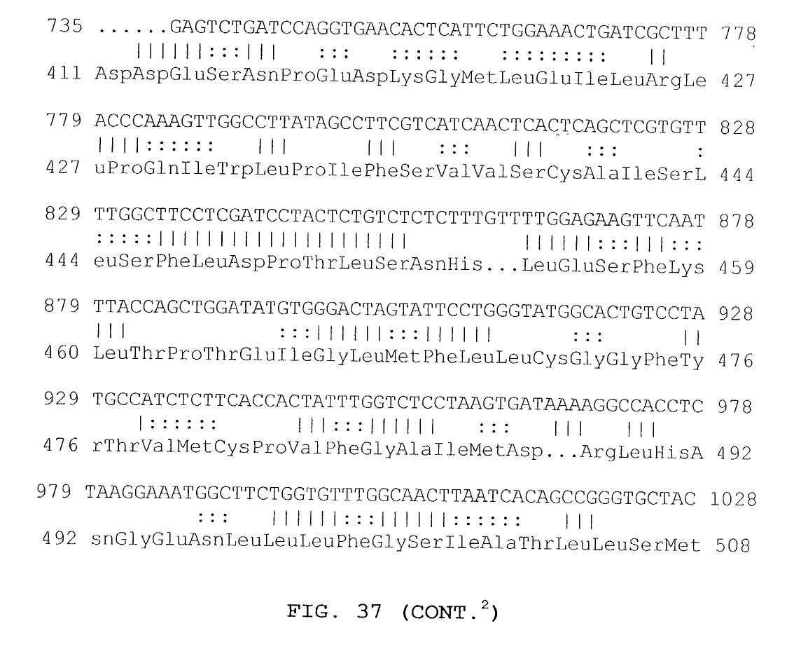

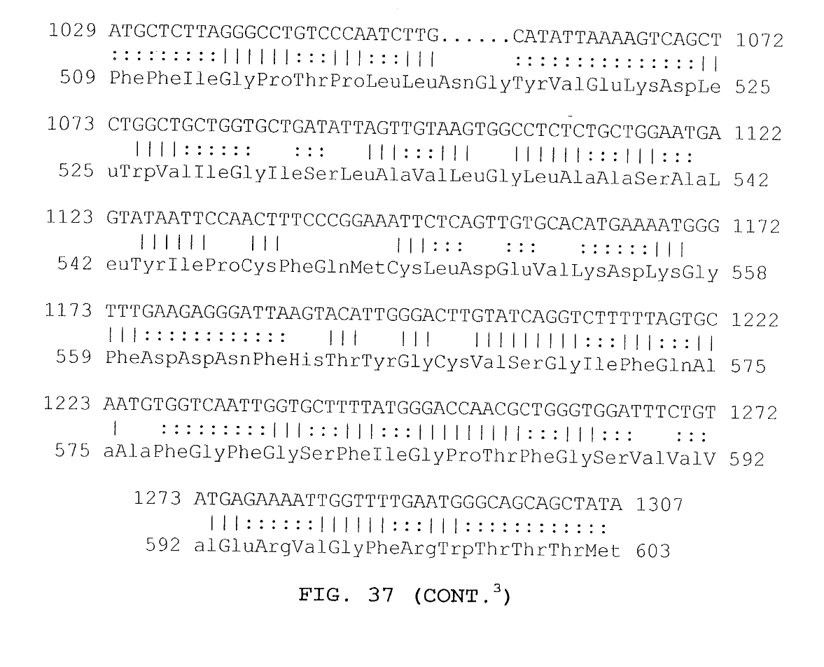

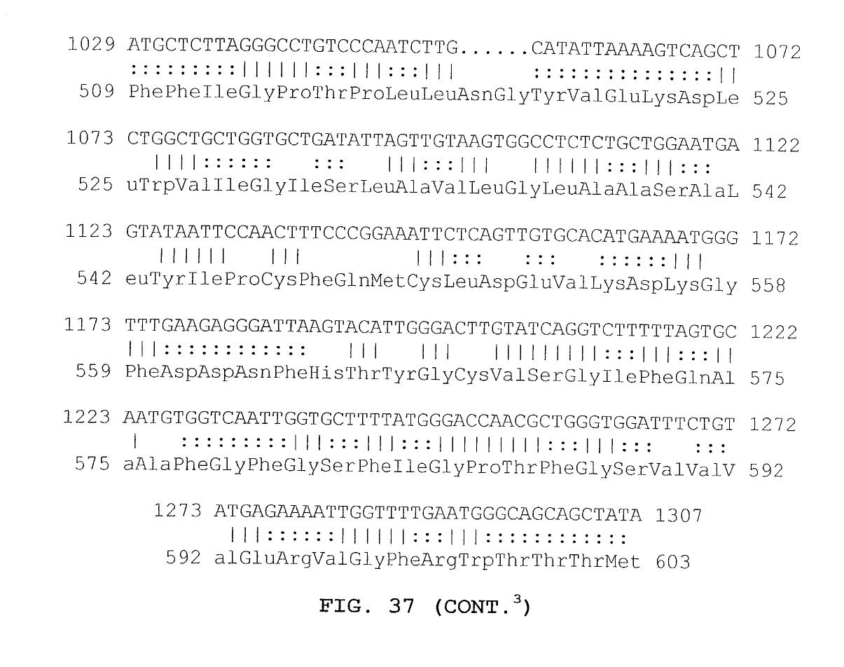

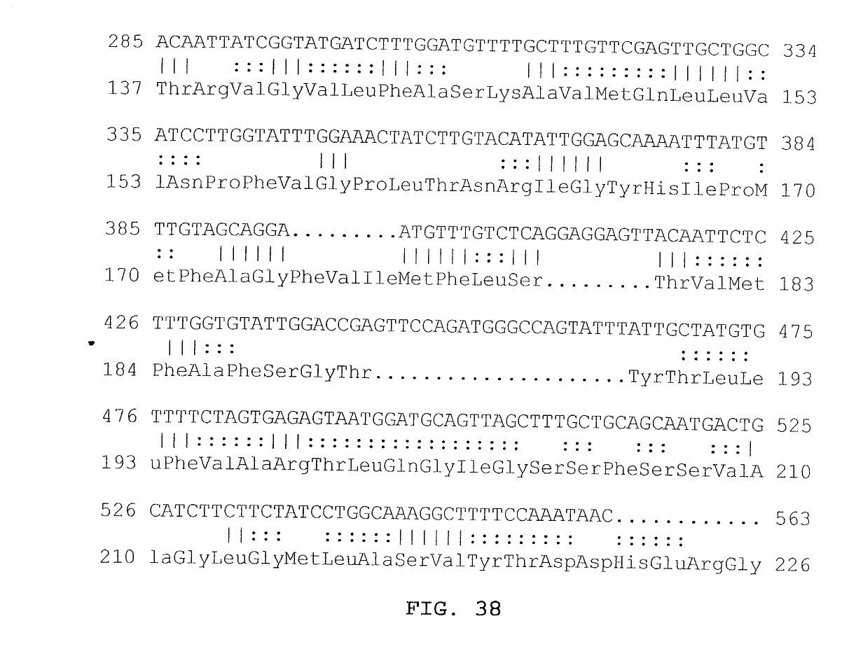

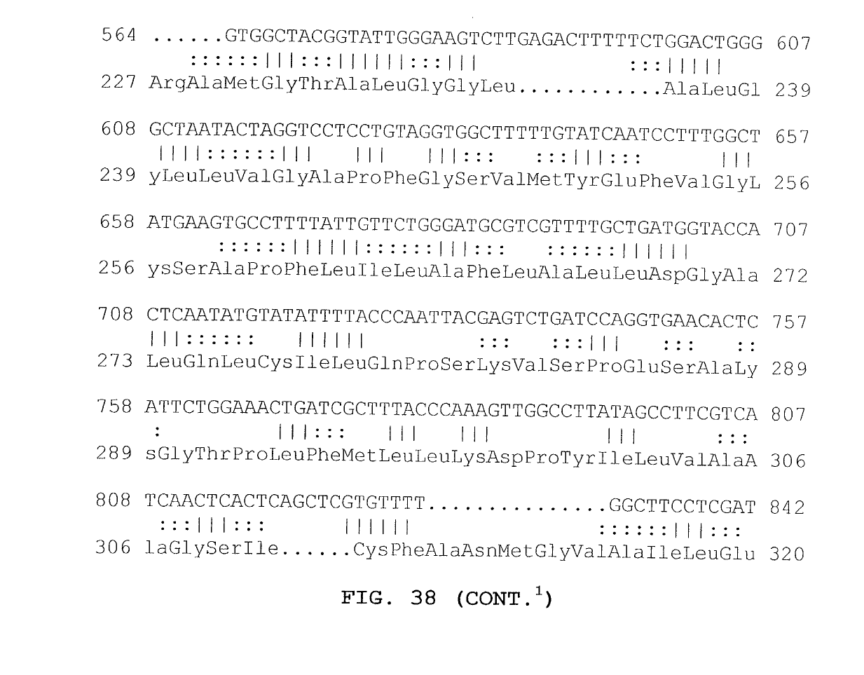

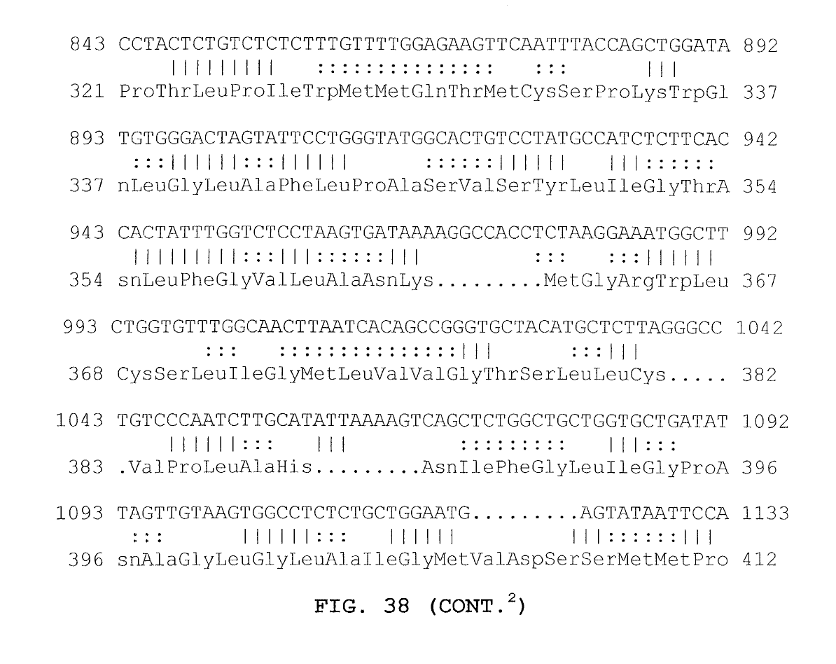

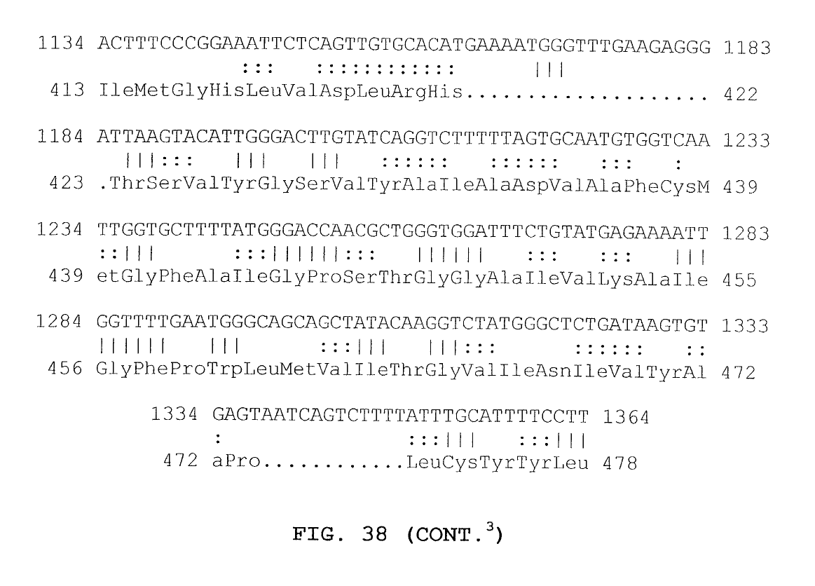

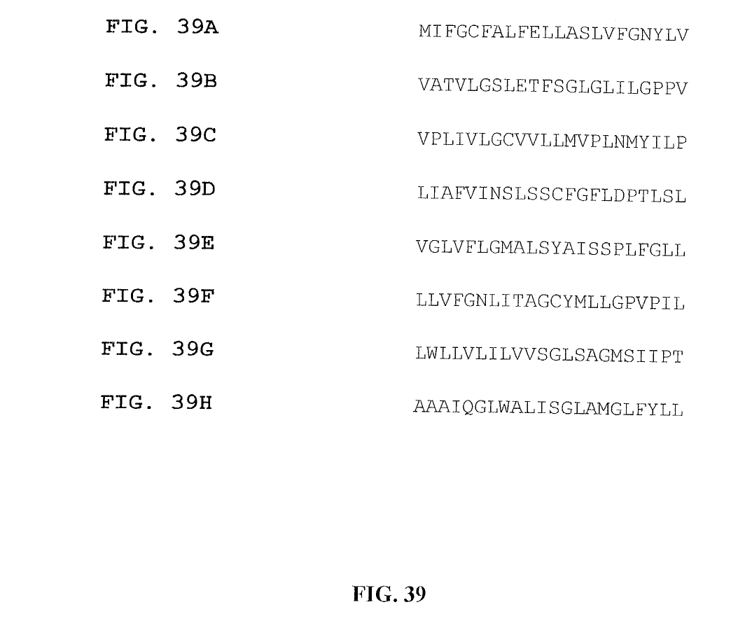

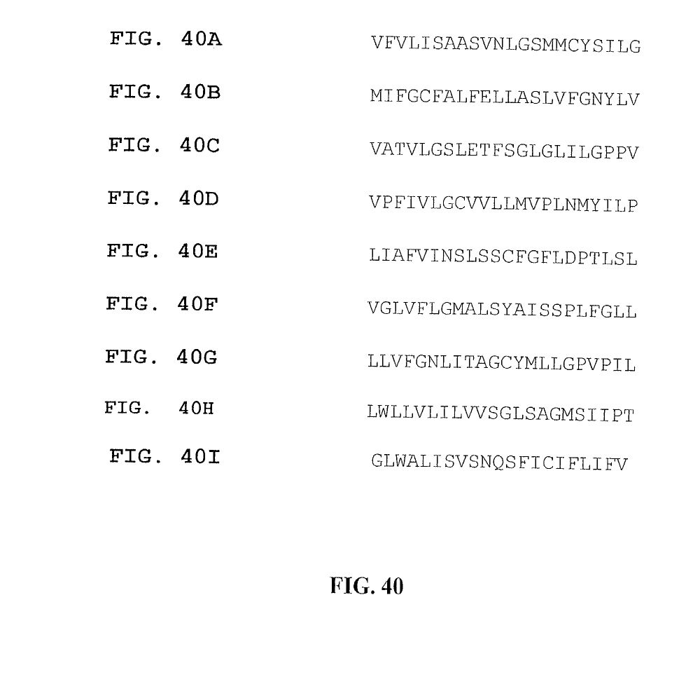

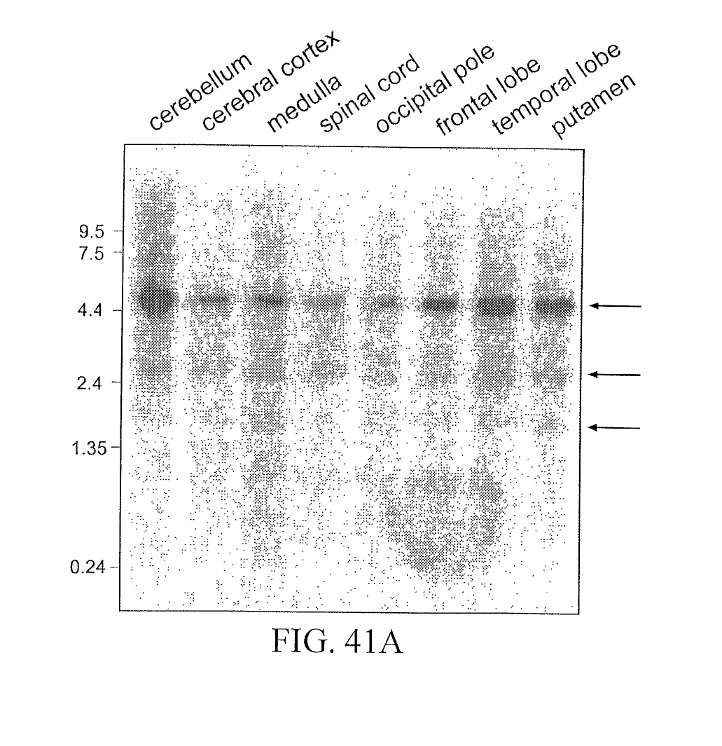

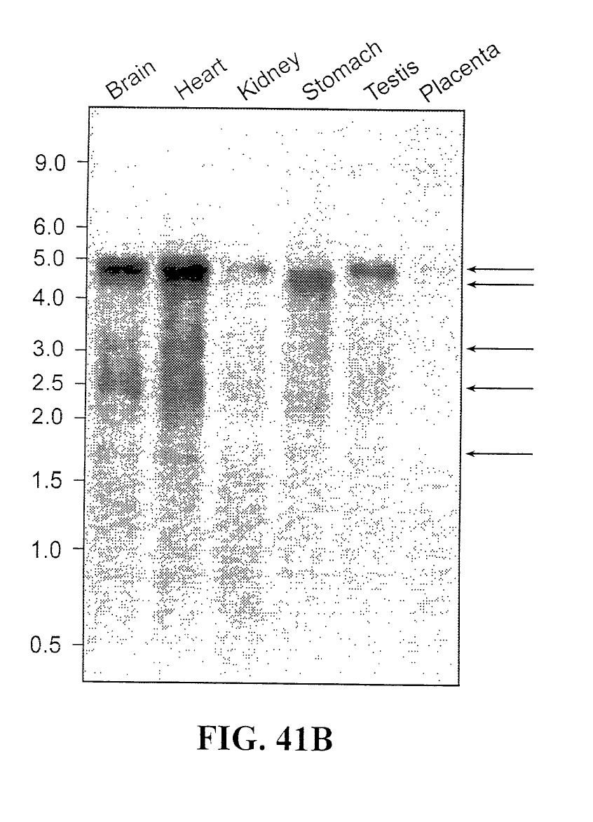

### DETAILED DESCRIPTION OF PREFERRED EMBODIMENTS

### EXAMPLE I

### Differences Between Variants and Original Sequences

The following is a table that compares the sequences of the variants of the invention to the original sequences from which they were varied and indicates here the variant differs from the original sequence. The terminology NV-1 to NV-28 corresponds to SEQ ID NO: 1 to SEQ ID NO: 28.

TABLE

| | New | |
|---|---|---|
| Protein | Variant | |
| Name | # | Description of the new variant |
| NEURO- | NV_1 | Insertion of an alternative exon of 15 amino |
| MODULIN | | acids after amino acid 171 of the original |
| | | protein |
| NEURO- | NV_2 | Alternative exon at 5′ end; alternative 39 |
| MODULIN | | amino acids instead of first 11 amino acids in |
| | | the original protein. |
| EDF-1 | NV_3 | Alternative exon at 3′ end of 7 amino acids |
| | | instead of 25 amino acids |
| EDF-1 | NV_4 | Alternative exon at 3′ end of 25 amino acids |
| | | instead of 76 amino acids |
| EDF-1 | NV_5 | Alternative exon at 3′ end of 13 amino acids |
| | | instead of 20 amino acids |
| Glucose | NV_6 | Alternative exon at 3′ end of 4 amino acids |
| transporter | | instead of 25 amino acids. Missing end of |
| glycoprotein | | CYTOPLASMIC domain |
| Glucose | NV_7 | Alternative exon at 3′ end of 22 amino acids |
| transporter | | instead of 13 amino acids in the end of |
| glycoprotein | | CYTOPLASMIC domain. |
| Glucose | NV_8 | Alternative exon at 3′ end of 23 amino acids |
| transporter | | instead of 44 amino acids in the end of |
| glycoprotein | | CYTOPLASMIC domain. |
| Glucose | NV_9 | Alternative exon at 3′ end of 7 amino acids |
| transporter | | instead of 44 amino acids in the end of |
| glycoprotein | | CYTOPLASMIC domain |
| Glucose | NV_10 | Alternative exon at 3′ end of 22 amino acids |
| transporter | | instead of 73 ammo acids in the end of |
| glycoprotein | | CYTOPLASMIC domain. Missing last |
| | | transmembrane domain. |
| B- | NV_11 | The variant has an alternative 5′ exon; 3 |
| LYMPHOCYTE | | amino acids instead of 191 amino acids. The |
| ANTIGEN CD20 | | variant is lacking 3 transmembrane domains, |
| | | out of possible 4 transmembrane domains. |
| | | The variant is also lacking 1 out 2 |
| | | cytoplasmic domains and all 3 disulfide |
| | | chains. |
| G1/S-SPECIFIC | NV_12 | Alternative exon at 3′ end; alternative 64 |
| CYCLIN D2 | | amino acids instead of 99 amino acids. |
| MELANOMA | NV_13 | Alternative exon at 3′ end of 1 amino acids |
| ANTIGEN | | instead of 21 amino acids |
| RECOGNIZED | | |
| BY T-CELLS 1 | | |
| MELANOMA | NV_14 | Alternative exon at 3′ end of 36 amino acids |
| ANTIGEN | | instead of 60 amino acids. |
| RECOGNIZED | | In the alternative exon is a predicted |
| BY T-CELLS 1 | | transmembrane domain |
| TYROSINE- | NV_15 | Alternative exon at 3′ end of 9 amino acids |
| PROTEIN | | instead of 348 ammo acids. The new variant |
| KINASE | | is missing a large portion of the cytoplasmic |
| RECEPTOR | | domain. The variant is also missing the |
| UFO | | entire PROTEIN KINASE domain, resulting |
| | | in a probable loss of its activity. |
| TUMOR | NV_16 | Deletion of 36 amino acids between |
| NECROSIS | | positions 173-210 |
| FACTOR, | | |
| ALPHA- | | |
| INDUCED | | |
| PROTEIN 2 (B94 | | |
| PROTEIN) | | |
| COMPLEMENT | NV_17 | Alternative exon at 3′ end of 32 amino acids |
| C5 | | instead of 457 amino acids. The new variant |
| | | is lacking the C5B (ALPHA′) domain, and is |
| | | missing the last potential glycosylation site. |
| COMPLEMENT | NV_18 | Alternative exon at 3′ end of 13 amino acids |
| C5 | | instead of 74 amino acids. The new variant is |
| | | lacking the end of the C5B (ALPHA′) |
| | | domain, and is missing the last potential |
| | | glycosylation site |
| T-CELL | NV_19 | Deletion of 55 amino acids between 241-296 |
| SURFACE | | |
| GLYCO- | | |
| PROTEIN | | |
| CD1B | | |
| TENASCIN | NV_20 | Deletion of 35 amino acids between 1879- |
| | | 1914. Missing small part of FIBRONECTIN |
| | | TYPE-III 15 |
| TNFR2- TRAF | NV_21 | Alternative exon at 3′ end of 6 amino acids |
| SIGNALING | | instead of 319 amino acids. The new variant |
| COMPLEX | | is lacking Zinc Finger and half of the third |
| PROTEIN 2 | | BIR repeat. Also, the new variant has two |
| | | SNIP in amino acid 235 and 241 |
| cytokine- | NV_22 | Alternative exon at 3′ end of 46 amino acids |
| inducible SH2 | | instead of 267 amino acids. |
| protein 6 | | |
| NEURONAL | NV_23 | Alternative exon at 5′ end of 35 amino acids |
| MEMBRANE | | instead of 22 amino acids |
| GLYCO- | | |
| PROTEIN M6-B | | |
| FIBROBLAST | NV_24 | Alternative exon at 5′ end of 31 amino acids |
| GROWTH | | instead of 67 amino acids. The new variant is |
| FACTOR | | missing the two BIPARTITE NUCLEAR |
| HOMOLOGOUS | | LOCALIZATION SIGNAL |
| FACTOR 1 | | |
| FIBRONECTIN | NV_25 | Deletion of 102 amino acids between 542- |
| RECEPTOR | | 644. The deletion is in the |
| BETA SUBUNIT | | EXTRACELLULAR domain; in the |
| INTEGRIN | | CYSTEINE-RICH REPEATS domain. The |
| BETA-1 | | new variant is lacking 1 (out of 12) potential |
| | | glycosylation sites |
| FIBRONECTIN | NV_26 | Deletion of 255 amino acids between 171- |
| RECEPTOR | | 427. The deletion is in the |
| BETA SUBUNIT | | EXTRACELLULAR domain. The new |
| INTEGRIN | | variant is lacking 5 (out of 12) potential |
| BETA-1 | | glycosylation sites |
| ENDOTHELIN B | NV_27 | Alternative exon at 3′ end of 13 amino acids |
| RECEPTOR | | instead of 268 amino acids The new variant |
| | | is missing 5 (out of 7) transmembrane |
| | | domains; missing 2 (out of 4) extracellular |
| | | domains; missing 3 (out of 4) cytoplasmic |
| | | domains; missing 1 (out of 1) disulfide |
| | | bonds; missing 3 (out of 3) PALMITATE |
| | | sites. |

### EXAMPLE II

### Designation of the Original Sequences, Therapeutical and Diagnostic Utilization of the Variant Sequences

Each novel variant of the invention is varied from an original sequence which has a known designation. The designation of the RNA sequences of the original sequences from which it was varied and the Accession Number of the original sequence are given below. First, information concerning the original sequence is given and then designation of the novel variants of the invention is given as NV-1 to NV-28 corresponding to SEQ ID NO: 1 to SEQ ID NO: 28.

The variants of SEQ ID NO: 1 to SEQ ID NO: 28 may be used to detect and treat a plurality of diseases or disorders stemming from malfunction of the original sequence.

### Neuromodulin

AXONAL MEMBRANE PROTEIN GAP-43

FUNCTION: THIS PROTEIN IS ASSOCIATED WITH NERVE GROWTH. IT IS A MAJOR COMPONENT OF THE MOTILE “GROWTH CONES” THAT FORM THE TIPS OF ELONGATING AXONS.

SUBCELLULAR LOCATION: CYTOPLASMIC SURFACE OF GROWTH CONE AND SYNAPTIC PLASMA MEMBRANES.

PTM: PHOSPHORYLATION OF THIS PROTEIN BY A PROTEIN KINASE C IS SPECIFICALLY CORRELATED WITH CERTAIN FORMS OF SYNAPTIC PLASTICITY.

MISCELLANEOUS: BINDS CALMODULIN WITH A GREATER AFFINITY IN THE ABSENCE OF CA++ THAN IN ITS PRESENCE

### NV_{—}1

Insertion of an alternative exon of 15 amino acids after amino acid 171.

This new exon is predicted as transmembrane domain. The new variant maintains all the necessary functional domains; phosphorylation sites, palmitate regions, and the domain important for membrane binding.

SEQ ID NO: 1 and sequences coded thereby may be used to detect or treat diseases relating to the central nervous system

### NV_{—}2

Alternative exon at 5′ end; alternative 39 amino acids instead of first 11 amino acids.

Missing 4 amino acids IMPORTANT FOR MEMBRANE BINDING, missing 2 lipid PALMITATE

SEQ ID NO. 2 and sequences coded thereby may be used to detect or treat diseases relating to the central nervous system.

### EDF-1 protein

A novel gene product down-regulated in human endothelial cell differentiation EDF-1 encodes a basic intracellular protein of 148 amino acids that is homologous to MBF1 (multiprotein-bridging factor 1) of the silkworm Bombyx mori and to H7, which is implicated in the early developmental events of Dictyostelium discoideum.

### NV_{—}3

Alternative exon at 3′ end of 7 amino acids instead of 25 amino acids.

### NV_{—}4

Alternative exon at 3′ end of 25 amino acids instead of 76 amino acids

### NV_{—}5

Alternative exon at 3′ end of 13 amino acids instead of 20 amino acids

All the above SEQ ID NOS: 3, 4, 5 and proteins coded thereby may be used to detect diseases concerning non-normal endothelial cells differentiation.

### Glucose Transporter Glycoprotein

FUNCTION: FACILITATIVE GLUCOSE TRANSPORTER. THIS ISOFORM MAY BE RESPONSIBLE FOR CONSTITUTIVE OR BASAL GLUCOSE UPTAKE. HAS A VERY BROAD SUBSTRATE SPECIFICITY; CAN TRANSPORT A WIDE RANGE OF ALDOSES INCLUDING BOTH PENTOSES AND HEXOSES.

SUBCELLULAR LOCATION: INTEGRAL MEMBRANE PROTEIN. TISSUE SPECIFICITY: EXPRESSED AT VARIABLE LEVELS IN MANY HUMAN TISSUES.

### NV_{—}6

Alternative exon at 3′ end of 4 amino acids instead of 25 amino acids. Missing end of CYTOPLASMIC domain

### NV_{—}7

Alternative exon at 3′ end of 22 amino acids instead of 13 amino acids in the end of CYTOPLASMIC domain.

### NV_{—}8

Alternative exon at 3′ end of 23 amino acids instead of 44 amino acids in the end of CYTOPLASMIC domain.

### NV_{—}9

Alternative exon at 3′ end of 7 amino acids instead of 44 amino acids in the end of CYTOPLASMIC domain.

### NV_{—}10

Alternative exon at 3′ end of 22 amino acids instead of 73 amino acids in the endof CYTOPLASMIC domain. Missing last transmembrane domain. All the above SEQ ID NOS. 6-10 and proteins coded thereby may be used to detect and treat diseases and disorders caused by non-normal glucose transport.

### B-LYMPHOCYTE ANTIGEN CD20

FUNCTION: THIS PROTEIN MAY BE INVOLVED IN THE REGULATION OF B-CELL ACTIVATION AND PROLIFERATION.

SUBCELLULAR LOCATION: INTEGRAL MEMBRANE PROTEIN.

PTM: PHOSPHORYLATED. MIGHT BE FUNCTIONALLY REGULATED BY PROTEIN KINASE(S).

### NV_{—}11

The variant has an alternative 5′ exon; 3 amino acids instead of 191 amino acids. The valiant is lacking 3 transmembrane domains, out of possible 4 transmembrane domains. The variant is also lacking 1 out 2 cytoplasmic domains and all 3 disulfide chains.

SEQ ID NO. 11 and proteins coded thereby may be used to treat and detect diseases involved in regulation of B-cell activation and proliferation, such as diseases involving the immune system.

### G1/S-SPECIFIC CYCLIN D2

FUNCTION: ESSENTIAL FOR THE CONTROL OF THE CELL CYCLE AT THE G1/S (START) TRANSITION. INTERACTS WITH THE CDC2 PROTEIN KINASE TO FORM MPF.

SIMILARITY: BELONGS TO THE CYCLIN FAMILY. CYCLIN D SUBFAMILY.

### NV_{—}12

Alternative exon at 3′ end; alternative 64 amino acids instead of 99 amino acids.

SEQ ID NO. 12 and proteins coded thereby can be used to treat or detect diseases involved in non-normal cell cycles, notably cancer diseases or degenerative diseases.

### MELANOMA ANTIGEN RECOGNIZED BY T-CELLS 1

TISSUE SPECIFICITY: EXPRESSION IS RESTRICTED TO MELANOMA AND MELANOCYTE CELL LINES AND RETINA

### NV_{—}13

Alternative exon at 3′ end of 1 amino acids instead of 21 amino acids.

### NV_{—}14

Alternative exon at 3′ end of 36 amino acids instead of 60 amino acids. In the alternative exon is a predicted transmembrane domain.

SEQ ID NO: 13 and SEQ ID NO: 14 and proteins coded thereby can be used to treat and detect melanoma, as well as to detect melanocyte cell lines.

### TYROSINE-PROTEIN KINASE RECEPTOR UFO (AXL ONCOGENE)

FUNCTION: MAY FUNCTION AS A SIGNAL TRANSDUCER BETWEEN SPECIFIC CELL TYPES OF MESODERMAL ORIGIN.

SUBCELLULAR LOCATION: TYPE I MEMBRANE PROTEIN.

DISEASE: HAS TRANSFORMING POTENTIAL IN PATIENTS WITH CHRONIC MYELOPROLIFERATIVE DISORDER OR CHRONIC MYELOCYTIC LEUKEMIA.

SIMILARITY: TO OTHER PROTEIN-TYROSINE KINASES IN THE CATALYTIC DOMAIN.

SIMILARITY: CONTAINS 2 IMMUNOGLOBULIN-LIKE C2-TYPE DOMAINS.

SIMILARITY: CONTAINS 2 FIBRONECTIN TYPE III-LIKE DOMAINS.

### NV_{—}15

Alternative exon at 3′ end of 9 amino acids instead of 348 amino acids. The new variant is missing a large portion of the cytoplasmic domain. The variant is also missing the entire PROTEIN KINASE domain, resulting in a probable loss of its activity.

SEQ ID NO: 15 and proteins coded thereby may be used to treat and detect diseases involving non-normal signal transduction especially in mesodermal cells.

### TUMOR NECROSIS FACTORS ALPHA-INDUCED PROTEIN 2 (B94 PROTEIN)

FUNCTION: MAY PLAY A ROLE AS A MEDIATOR OF INFLAMMATION AND ANGIOGENESIS.

DEVELOPMENTAL STAGE: DIFFERENTIALLY EXPRESSED IN DEVELOPMENT AND CAPILLARY TUBE-LIKE FORMATION IN VITRO.

INDUCTION: BY TNF AND OTHER PROINFLAMMATORY FACTORS.

### NV_{—}16

Deletion of 36 amino acids between positions 173-210.

SEQ ID NO. 10 and sequences coded thereby may be used to treat and detect diseases involving the immune system, such as inflammatory diseases, autoimmune diseases and cancer.

### COMPLEMENT C5

FUNCTION: ACTIVATION OF C5 BY A C5 CONVERTASE INITIATES THE SPONTANEOUS ASSEMBLY OF THE LATE COMPLEMENT COMPONENTS, C5-C9, INTO THE MEMBRANE ATTACK COMPLEX. C5B HAS A TRANSIENT BINDING SITE FOR C6. THE C5B-C6 COMPLEX IS THE FOUNDATION UPON WHICH THE LYTIC COMPLEX IS ASSEMBLED.

FUNCTION: DERIVED FROM PROTEOLYTIC DEGRADATION OF COMPLEMENT C5, C5 ANAPHYLATOXIN IS A MEDIATOR OF LOCAL INFLAMMATORY PROCESS. IT INDUCES THE CONTRACTION OF SMOOTH MUSCLE, INCREASES VASCULAR PERMEABILITY AND CAUSES HISTAMINE RELEASE FROM MAST CELLS AND BASOPHILIC LEUKOCYTES. C5A ALSO STIMULATES THE LOCOMOTION OF POLYMORPHONUCLEAR LEUKOCYTES (CHEMOKINESIS) AND DIRECT THEIR MIGRATION TOWARD SITES OF INFLAMMATION (CHEMOTAXIS).

SUBUNIT: C5 PRECURSOR IS FIRST PROCESSED BY THE REMOVAL OF 4 BASIC RESIDUES, FORMING TWO CHAINS, BETA & ALPHA, LINKED BY A DISULFIDE BOND. C5 CONVERTASE ACTIVATES C5 BY CLEAVING THE ALPHA CHAIN, RELEASING C5A ANAPHYLATOXIN & GENERATING C5B (BETA CHAIN+ALPHA′ CHAIN).

SIMILARITY: TO C3, C4 AND ALPHA-2-MACROGLOBULIN.

SIMILARITY: CONTAINS 1 ANAPHYLATOXIN-LIKE DOMAIN.

### NV_{—}17

Alternative exon at 3′ end of 32 amino acids instead of 457 amino acids. The new variant is lacking the C5B (ALPHA′) domain, and is missing the last potential glycosylation site.

SEQ ID NO. 17 and SEQ ID NO. 18 and sequences coded thereby may be used to detect and treat diseases involved in non-normal complement activity.

### NV_{—}18

Alternative exon at 3′ end of 13 amino acids instead of 74 amino acids. The new variant is lacking the end of the C5B (ALPHA′) domain, and is missing the last potential glycosylation site.

### T-CELL SURFACE GLYCOPROTEIN CD1B

FUNCTION: NOT KNOWN.

SUBUNIT: ASSOCIATES NON-COVALENTLY WITH BETA-2- MICRO-GLOBULIN.

SUBCELLULAR LOCATION: TYPE I MEMBRANE PROTEIN.

TISSUE SPECIFICITY: EXPRESSED ON CORTICAL THYMOCYTES, ON CERTAIN T-CELL LEUKEMIAS, AND IN VARIOUS OTHER TISSUES.

SIMILARITY: BELONGS TO THE IMMUNOGLOBULIN SUPERFAMILY.

### NV_{—}19

Deletion of 55 amino acids between 241-296.

SEQ ID NO. 19 and proteins coded thereby may be used to treat and detect diseases concerning globulines, in particular immune-related diseases.

### TENASCIN

FUNCTION: SAM (SUBSTRATE-ADHESION MOLECULE) THAT APPEARS TO INHIBIT CELL MIGRATION. MAY PLAY A ROLE IN SUPPORTING THE GROWTH OF EPITHELIAL TUMORS.

SUBUNIT: HEXAMERIC. A HOMOTRIMER MAY BE FORMED IN THE TRIPLE COILED-COIL REGION AND MAY BE STABILIZED BY DISULFIDE RINGS AT BOTH ENDS. TWO OF SUCH HALF-HEXABRACHIONS MAY BE DISULFIDE LINKED WITHIN THE CENTRAL GLOBULE.

SUBCELLULAR LOCATION: EXTRACELLULAR MATRIX.

ALTERNATIVE PRODUCTS: FOUR VARIANTS ARE PRODUCED FROM A SINGLE GENE IN A TISSUE- AND TIME-SPECIFIC MANNER DURING DEVELOPMENT.

INDUCTION: BY TGF-BETA.

SIMILARITY: CONTAINS 15 EGF-LIKE DOMAINS.

SIMILARITY: CONTAINS 15 FIBRONECTIN TYPE III-LIKE DOMAINS.

SIMILARITY: CONTAINS 1 FIBRINOGEN-LIKE DOMAIN.

### NV_{—}20

Deletion of 35 amino acids between 1879-1914. Missing small part of FIBRONECTIN TYPE-III 15

SEQ ID NO. 20 and sequences encoded thereby may be used to treat epithelial tumors.

### TNFR2-TRAF SIGNALING COMPLEX PROTEIN 2

FUNCTION: APOPTOTIC SUPPRESSOR. THE BIR MOTIFS REGION INTERACTS WITH TNF RECEPTOR ASSOCIATED FACTORS 1 AND 2 (TRAF1 AND TRAF2) TO FORM AN HETEROMERIC COMPLEX, WHICH IS THEN RECRUITED TO THE TUMOR NECROSIS FACTOR RECEPTOR 2 (TNFR2).

SUBCELLULAR LOCATION: CYTOPLASMIC (POTENTIAL).

TISSUE SPECIFICITY: PRESENT IN MANY FETAL AND ADULT TISSUES. MAINLY EXPRESSED IN ADULT SKELETAL MUSCLE, THYMUS, TESTIS, OVARY, AND PANCREAS, LOW OR ABSENT IN BRAIN AND PERIPHERAL BLOOD LEUKOCYTES.

SIMILARITY: BELONGS TO THE IAP FAMILY.

SIMILARITY: CONTAINS 3 BIR DOMAINS (BACULOVIRAL INHIBITION OF APOPTOSIS PROTEIN REPEAT).

SIMILARITY: CONTAINS A C3HC4-CLASS ZINC FINGER.

### NV_{—}21

Alternative exon at 3′ end of 6 amino acids instead of 319 amino acids. The new variant is lacking Zinc Finger and half of the third BIR repeat. Also, the new variant has two SNIP in amino acid 235 and 241.

SEQ ID NO. 21 may be used to suppress apoptosis for example in neurodegenerative diseases. Antibodies and complementary sequences may be used in case apoptosis is to be encouraged, such as in cancer.

### CYTOKINE-INDUCIBLE SH2 PROTEIN 6

### NV_{—}22

Alternative exon at 3′ end of 46 amino acids instead of 267 amino acids.

### NEURONAL MEMBRANE GLYCOPROTEIN M6-B

FUNCTION: MAY BE INVOLVED IN NEURAL DEVELOPMENT.

SUBCELLULAR LOCATION: INTEGRAL MEMBRANE PROTEIN (BY SIMILARITY).

TISSUE SPECIFICITY: NEURONS AND GLIA; CEREBELLAR BERGMANN GLIA, IN GLIA WITHIN WHITE MATTER TRACTS OF THE CEREBELLUM AND CEREBRUM, AND IN EMBRYONIC DORSAL ROOT GANGLIA.

SIMILARITY: BELONGS TO THE MYELIN PROTEOLIPID PROTEIN FAMILY.

### NV_{—}23

Alternative exon at 5′ end of 35 amino acids instead of 22 amino acids. SEQ ID NO. 23 and proteins encoded thereby may be used to detect and treat neurodegenerative diseases.

### FIBROBLAST GROWTH FACTOR HOMOLOGOUS FACTOR 1

FUNCTION: PROBABLY INVOLVED IN NERVOUS SYSTEM DEVELOPMENT AND FUNCTION.

SUBCELLULAR LOCATION: NUCLEAR (PROBABLE).

TISSUE SPECIFICITY: BRAIN, EYE AND TESTIS; HIGHLY EXPRESSED IN EMBRYONIC RETINA, OLFACTORY EPITHELIUM, OLFACTORY BULB, AND IN A SEGMENTAL PATTERN OF THE BODY WALL; IN ADULT OLFACTORY BULB, LESS IN CEREBELLUM, DEEP CEREBELLAR NUCLEI, CORTEX, AND MULTIPLE MIDBRAIN STRUCTURES.

SIMILARITY: BELONGS TO THE HEPARIN-BINDING GROWTH FACTORS FAMILY.

### NV_{—}24

Alternative exon at 5′ end of 31 amino acids instead of 67 amino acids. The new variant is missing the two BIPARTITE NUCLEAR LOCALIZATION SIGNAL. SEQ ID NO. 24 and sequences encoded thereby may be used to detect and treat neurodegenerative diseases.

### FIBRONECTIN RECEPTOR BETA SUBUNIT INTEGRIN BETA-1

FUNCTION: ASSOCIATES WITH ALPHA-1 OR ALPHA-6 TO FORM A LAMININ RECEPTOR, WITH ALPHA-2 TO FORM A COLLAGEN RECEPTOR, WITH ALPHA-4 TO INTERACT WITH VCAM-1, WITH ALPHA-5 TO FORM A FIBRONECTIN RECEPTOR AND WITH ALPHA-8. INTEGRINS RECOGNIZE THE SEQUENCE R-G-D IN THEIR LIGAND.

SUBUNIT: DIMER OF AN ALPHA AND BETA SUBUNIT. THE BETA-1 CHAIN IS KNOWN TO ASSOCIATE WITH ALPHA-1, -2, -3, -4, -5, -6, -7, -8, -9, AND -V.

SUBCELLULAR LOCATION: TYPE I MEMBRANE PROTEIN.

PTM: THE CYSTEINE RESIDUES ARE INVOLVED IN INTRACHAIN DISULFIDE BONDS.

SIMILARITY: BELONGS TO THE INTEGRIN BETA CHAIN FAMILY.

### NV_{—}25

Deletion of 102 amino acids between 542-644. The deletion is in the EXTRACELLULAR domain; in the CYSTEINE-RICH REPEATS domain. The new variant is lacking 1 (out of 12) potential glycosylation sites.

### NV_{—}26

Deletion of 255 amino acids between 171-427. The deletion is in the EXTRACELLULAR domain. The new valiant is lacking 5 (out of 12) potential glycosylation sites.

SEQ ID NO. 25 and SEQ ID NO. 26 encoded thereby may be used to treat and detect conditions involving collagen disorders, and various forms of fibrosis.

### ENDOTHELIN B RECEPTOR

FUNCTION: NON-SPECIFIC RECEPTOR FOR ENDOTHELIN 1, 2, AND 3. MEDIATES ITS ACTION BY ASSOCIATION WITH G PROTEINS THAT ACTIVATE A PHOSPHATIDYLINOSITOL-CALCIUM SECOND MESSENGER SYSTEM.

SUBCELLULAR LOCATION: INTEGRAL, MEMBRANE PROTEIN.

DISEASE: DEFECTS IN EDNRB ARE A CAUSE OF TYPE IV (WS4 OR SHAH- WAARDENBURG SYNDROME) (WS/HSCR) WHICH IS CHARACTERIZED BY THE ASSOCIATION OF WS AND HIRSCHSPRUNG DISEASE (HSCR).

DISEASE: DEFECTS IN EDNRB ARE THE CAUSE OF TYPE 2 HIRSCHSPRUNG DISEASE (HSCR2) (OR AGANGLIONIC MEGACOLON), A CONGENITAL DISORDER CHARACTERIZED BY ABSENCE OF ENTERIC GANGLIA ALONG A VARIABLE LENGTH OF THE INTESTINE. HSCR IS THE MOST COMMON CAUSE OF CONGENITAL INTESTINAL OBSTRUCTION EARLY SYMPTOMS RANGE FROM COMPLETE ACUTE NEONATAL OBSTRUCTION, CHARACTERIZED BY VOMITING, ABDOMINAL DISTENTION AND FAILURE TO PASS STOOL, TO CHRONIC CONSTIPATION IN THE OLDER CHILD.

### NV_{—}27

Alternative exon at 3′ end of 13 amino acids instead of 268 amino acids. The new variant is missing 5 (out of 7) transmembrane domains; missing 2 (out of 4) extracellular domains; missing 3 (out of 4) cytoplasmic domains; missing 1 (out of 1) disulfide bonds; missing 3 (out of 3) PALMITATE sites.

SEQ ID NO. 27 and sequences encoded thereby may be used to treat and detect Type IV (WS4 or Shahwaaardenburg syndrome and Hirschsprung disease.

### RAS-LIKE PROTEIN

### NV_{—}28

Deletion of 86 amino acids between 75-161.

SEQ ID. NO. 28 and sequences encoded thereby may be used to detect and treat cancer.

### EXAMPLE III

### Variant, TL and TH Nucleic Acid Sequence

The nucleic acid sequences of the invention include nucleic acid sequences which encode variant product and fragments and analogs thereof as well as nucleic acid sequences which code for the TL or the TH products. The nucleic acid sequences may alternatively be sequences complementary to the above coding sequences, or to regions of said coding sequence. The length of the complementary sequence is sufficient to avoid the expression of the coding sequence. The nucleic acid sequences may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand. The nucleic acid sequences may also both include dNTPs, rNTPs as well as non naturally occurring sequences. The sequence may also be a pail of a hybrid between an amino acid sequence and a nucleic acid sequence.

In a general embodiment, the nucleic acid sequence has at least 90%, identity with any one of the sequence identified as SEQ ID NO: 1 to SEQ ID NO: 41 provided that this sequence is not completely identical with that of the original sequence. In another general embodiment the nucleic acid sequence has at least 70% identity, preferably 80% identity, most preferably 90% identity with any of the sequences of SEQ ID NO: 1 to SEQ ID NO: 41.

The nucleic acid sequences may include the coding sequence by itself. By another alternative the coding region may be in combination with additional coding sequences, such as those coding for fusion protein or signal peptides, in combination with non-coding sequences, such as introns and control elements, promoter and terminator elements or 5′ and/or 3 untranslated regions, effective for expression of the coding sequence in a suitable host, and/or in a vector or host environment in which any of the above nucleic acid sequence is introduced as a heterologous sequence.

The nucleic acid sequences of the present invention may also have the product coding sequence fused in-frame to a marker sequence which allows for purification of the variant product. The marker sequence may be, for example, a hexahistidine tag to provide for purification of the mature polypeptide fused to the marker in the case of a bacterial host, or, the marker sequence may be a hemagglutinin (HA) tag when a mammalian host, e.g. COS-7 cells, is used. The HA tag corresponds to an epitope derived from the influenza hemagglutinin protein (Wilson, I., et al. Cell 37:767 (1984)).

Also included in the scope of the invention are fragments as defined above also referred to herein as oligonucleotides, typically having at least 20 bases, preferably 20-30 bases corresponding to a region of the coding-sequence nucleic acid sequence. The fragments may be used as probes, primers, and when complementary also as antisense agents, and the like, according to known methods.

As indicated above, the nucleic acid sequence may be substantially a depicted in any one of SEQ ID NO: 1 to SEQ ID NO: 41 or fragments thereof or sequences having at least 90% identity to the above sequence as explained above, or sequences having at least 70%, preferably 80%, most preferably 90% identity to the above sequences. Alternatively, due to the degenerative nature of the genetic code, the sequence may be a sequence coding for any one of the amino acid sequence of SEQ ID NO: 42 to SEQ ID NO: 81, or fragments or analogs of said amino acid sequence.

A. Preparation of Nucleic Acid Sequences

The nucleic acid sequences may be obtained by screening cDNA libraries using oligonucleotide probes which can hybridize to or PCR-amplify nucleic acid sequences which encode any one of the products disclosed above. cDNA libraries prepared from a variety of tissues are commercially available and procedures for screening and isolating cDNA clones are well-known to those of skill in the art. Such techniques are described in, for example, Sambrook et al. (1989) Molecular Cloning: A Laboratory Manual (2nd Edition), Cold Spring Harbor Press, Plainview, N.Y and Ausubel F M et al. (1989) Current Protocols in Molecular Biology, John Wiley & Sons, New York, N.Y.

The nucleic acid sequences may be extended to obtain upstream and downstream sequences such as promoters, regulatory elements, and 5′ and 3′ untranslated regions (UTRs). Extension of the available transcript sequence may be performed by numerous methods known to those of skill in the art such as PCR or primer extension (Sambrook et al., supra), or by the RACE method using, for example, the Marathon RACE kit (Clontech, Cat. #K1802-1).

Alternatively, the technique of “restriction-site” PCR (Gobinda et al. PCR Methods Applic. 2:318-22, (1993)), which uses universal primers to retrieve flanking sequence adjacent a known locus, may be employed. First, genomic DNA is amplified in the presence of primer to a linker sequence and a primer specific to the known region. The amplified sequences are subjected to a second round of PCR with the same linker primer and another specific primer internal to the first one. Products of each round of PCR are transcribed with an appropriate RNA polymerase and sequenced using reverse transcriptase.

Inverse PCR can be used to amplify or extend sequences using divergent primers based on a known region (Triglia, T. et al., Nucleic Acids Res. 16:8186, (1988)). The primers may be designed using OLIGO(R) 4.06 Primer Analysis Software (1992; National Biosciences Inc, Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Capture PCR (Lagerstrom, M. et al., PCR Methods Applic. 1:111-19, (1991)) is a method for PCR amplification of DNA fragments adjacent to a known sequence in human and yeast artificial chromosome DNA. Capture PCR also requires multiple restriction enzyme digestions and ligations to place an engineered double-stranded sequence into a flanking part of the DNA molecule before PCR.

Another method which may be used to retrieve flanking sequences is that of Parker, J. D., et al., Nucleic Acids Res., 19:3055-60, (1991)). Additionally, one can use PCR, nested primers and PromoterFinder™ libraries to “walk in” genomic DNA (PromoterFinder™; Clontech, Palo Alto, Calif.). This process avoids the need to screen libraries and is useful in finding intron/exon junctions. Preferred libraries for screening for full length cDNAs are ones that have been size-selected to include larger cDNAs. Also, random primed libraries are preferred in that they will contain more sequences which contain the 5′ and upstream regions of genes.

A randomly primed library may be particularly useful if an oligo d(T) library does not yield a full-length cDNA. Genomic libraries are useful for extension into the 5′ nontranslated regulatory region.

The nucleic acid sequences and oligonucleotides of the invention can is also be prepared by solid-phase methods, according to known synthetic methods. Typically, fragments of up to about 100 bases are individually synthesized, then joined to form continuous sequences up to several hundred bases.

B. Use of Nucleic Acid Sequence for the Production of Products

In accordance with the present invention, nucleic acid sequences specified above may be used as recombinant DNA molecules that direct the expression of any of the products of the invention (i.e. the variant products, the TL products or the TH products).

As will be understood by those of skill in the art, it may be advantageous to produce product-encoding nucleotide sequences possessing codons other than those which appear in any one of SEQ ID NO: 1 to SEQ ID NO: 41 which are those which naturally occur in the human genome. Codons preferred by a particular prokaryotic or eukaryotic host (Murray, E. et al. Nuc Acids Res., 17:477-508, (1989)) can be selected, for example, to increase the rate of product expression or to produce recombinant RNA transcripts having desirable properties, such as a longer half-life, than transcripts produced from naturally occurring sequences.

The nucleic acid sequences of the present invention can be engineered in order to alter the product coding sequence for a variety of reasons, including but not limited to, alterations which modify the cloning, processing and/or expression of the product. For example, alterations may be introduced using techniques which are well known in the art, e.g., site-directed mutagenesis, to insert new restriction sites, to alter glycosylation patterns, to change codon preference, etc.

The present invention also includes recombinant constructs comprising one or more of the sequences as broadly described above. The constructs comprise a vector, such as a plasmid or viral vector, into which a nucleic acid sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. Appropriate cloning and expression vectors for use with prokaryotic and eukaryotic hosts are also described in Sambrook, et al., (supra).

The present invention also relates to host cells which are genetically engineered with vectors of the invention, and the production of the product of the invention by recombinant techniques. Host cells are genetically engineered (i.e., transduced, transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the expression of the valiant nucleic acid sequence. The culture conditions, such as temperature, pH and the like, are those previously used with the host cell selected for expression, and will be apparent to those skilled in the art.

The nucleic acid sequences of the present invention may be included in any one of a variety of expression vectors for expressing a product. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However; any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and related sub-cloning procedures are deemed to be within the scope of those skilled in the art.

The DNA sequence in the expression vector is operatively linked to an appropriate transcription control sequence (promoter) to direct mRNA synthesis. Examples of such promoters include: LTR or SV40 promoter, the E. coli lac or trp promoter, the phage lambda PL promoter, and other promoters known to control expression of genes in prokaryotic or eukaryotic cells or their viruses. The expression vector also contains a ribosome binding site for translation initiation, and a transcription terminator The vector may also include appropriate sequences for amplifying expression. In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as described above, as well as an appropriate promoter or control sequence, may be employed to transform an appropriate host to permit the host to express the protein. Examples of appropriate expression hosts include: bacterial cells, such as E. coli, Streptomyces, Salmonella typhimurium; fungal cells, such as yeast; insect cells such as Drosophila and Spodoptera Sf9; animal cells such as CHO, COS, HEK 293 or Bowes melanoma; adenoviruses; plant cells, etc. The selection of an appropriate host is deemed to be within the scope of those skilled in the art from the teachings herein. The invention is not limited by the host cells employed.

In bacterial systems, a number of expression vectors may be selected depending upon the use intended for any of the products of the invention. For example, when large quantities of product are needed for the induction of antibodies, vectors which direct high level expression of fusion proteins that are readily purified may be desirable. Such vectors include, but are not limited to, multifunctional E.coli cloning and expression vectors such as Bluescript(R) (Stratagene), in which the polypeptide coding sequence may be ligated into the vector in-frame with sequences for the amino-terminal Met and the subsequent 7 residues of beta-galactosidase so that a hybrid protein is produced; pIN vectors (Van Heeke & Schuster J Biol Chem. 264:5503-5509, (1989)); pET vectors (Novagen, Madison Wis.); and the like.

In the yeast Saccharomyces cerevisiae a number of vectors containing constitutive or inducible promoters such as alpha factor, alcohol oxidase and PGH may be used. For reviews, see Ausubel et al. (supra) and Grant et al., (Methods in Enzymology 153:516-544, (1987)).

In cases where plant expression vectors are used, the expression of a sequence encoding any of the products of the invention may be driven by any of a number of promoters. For example, viral promoters such as the 35S and 19S promoters of CaMV(Bnisson et al., Nature 310:511-514.(1984)) may be used alone or in combination with the omega leader sequence from TMV (Takamatsu et al., EMBO J., 6:307-311, (1987)). Alternatively, plant promoters such as the small subunit of RUBISCO (Coruzzi et al., EMBO J 3:1671-1680, (1984); Broglie et al., Science 224:838-843, (1984)); or heat shock promoters (Winter J and Sinibaldi R. M., Results Probl. Cell Differ., 17:85-105, (1991)) may be used. These constructs can be introduced into plant cells by direct DNA transformation or pathogen-mediated transfection. For reviews of such techniques, see Hobbs S. or Murry L. E. (1992) in McGraw Hill Yearbook of Science and Technology, McGraw Hill, New York, N.Y., pp 191-196; or Weissbach and Weissbach (1988) Methods for Plant Molecular Biology, Academic Press, New York, N.Y., pp 421-463.

The products of the invention may also be expressed in an insect system. In one such system, Autographa californica nuclear polyhedrosis virus (AcNPV) is used as a vector to express foreign genes in Spodoptera frugiperda cells or in Trichoplusia larvae. The product coding sequence may be cloned into a nonessential region of the virus, such as the polyhedrin gene, and placed under control of the polyhedrin promoter. Successful insertion of coding sequence will render the polyhedrin gene inactive and produce recombinant virus lacking coat protein coat. The recombinant viruses are then used to infect S. frugiperda cells or Trichoplusia larvae in which valiant protein is expressed (Smith et al., J. Virol. 46:584, (1983); Engelhard, E. K. et al., Proc. Nat. Acad. Sci. 91:3224-7, (1994)).

In mammalian host cells, a number of viral-based expression systems may be utilized. In cases where an adenovirus is used as an expression vector, a product coding sequence may be ligated into an adenovirus transcription/translation complex consisting of the late promoter and tripartite leader sequence. Insertion in a nonessential E1 or E3 region of the viral genome will result in a viable virus capable of expressing any one of the products of the invention in infected host cells (Logan and Shenk, Proc. Natl. Acad. Sci. 81:3655-59, (1984). In addition, transcription enhancers, such as the Rous sarcoma virus (RSV) enhancer, may be used to increase expression in mammalian host cells.

Specific initiation signals may also be required for efficient translation of products coding sequence. These signals include the ATG initiation codon and adjacent sequences. In cases where the product coding sequence, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression may be enhanced by the inclusion of enhancers appropriate to the cell system in use (Scharf, D. et al., (1994) Results Probl. Cell Differ., 20:125-62, (1994); Bittner et al., Methods in Enzymol 153:516-544, (1987)).

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. (1986) Basic Methods in Molecular Biology). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre-pro” form of the protein may also be important for collect insertion, folding and/or function. Different host cells such as CHO, HeLa, MDCK, 293, WI38, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express variant product may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler M., et al., Cell 11:223-32, (1977)) and adenine phosphoribosyltransferase (Lowy I., et al., Cell 22:817-23, (1980)) genes which can be employed in tk- or aprt- cells, respectively. Also, antimetabolite, antibiotic or herbicide resistance can be used as the basis for selection; for example, dhfr which confers resistance to methotrexate (Wigler M., et al., Proc. Natl Acad. Sci. 77:3567-70, 1(1980)); npt, which confers resistance to the aminoglycosides neomycin and G-418 (Colbere-Garapin, F. et al., J Mol. Biol., 150:1-14, (1981)) and als or pat, which confer resistance to chlorsulfuron and phosphinotricin acetyltransferase, respectively (Munry, supra). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad Sci. 85:8047-51, (1988)). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes, C. A. et. al., Methods Mol. Biol., 55:121-131, (1995)).

Host cells transformed with a nucleotide sequence encoding any one of the products of the invention may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors containing nucleic acid sequences encoding any one of the products of the invention can be designed with signal sequences which direct secretion of the product through a prokaryotic or eukaryotic cell membrane.

The product of the invention may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, Seattle, Wash.). The inclusion of a protease-cleavable polypeptide linker sequence between the purification domain and the product is useful to facilitate purification. One such expression vector provides for expression of a fusion protein compromising a product of the invention fused to a polyhistidine region separated by an enterokinase cleavage site. The histidine residues facilitate purification on IMIAC (immobilized metal ion affinity chromatography, as described in Porath, et al., Protein Expression and Purification, 3:263-281, (1992)) while the enterokinase cleavage site provides a means for isolating products of the invention from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

The products of the invention can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps.

C. Diagnostic Applications Utilizing Nucleic Acid Sequences

The nucleic acid sequences of the present invention may be used for a variety of diagnostic purposes. The nucleic acid sequences may be used to detect and quantitate expression of the sequences of the invention in patient's cells, e.g. biopsied tissues, by detecting the presence of mRNA coding for any one of the products of the invention. Alternatively, the assay may be used to detect soluble products of the invention in the serum or blood. This assay typically involves obtaining total mRNA from the tissue, blood or serum and contacting the mRNA with a nucleic acid probe. The probe is a nucleic acid molecule of at least 20 nucleotides, preferably 20-30 nucleotides, capable of specifically hybridizing with a sequence included within the sequence of a nucleic acid molecule encoding any one of the products of the invention under hybridizing conditions, detecting the presence of mRNA hybridized to the probe, and thereby detecting the expression of any one of the nucleic acid sequences of the invention. This assay can be used to distinguish between absence, presence, and excess expression of the product and to monitor levels of expression of any one of the nucleic acid sequences during therapeutic intervention. In addition, the assay may be used to compare the levels of any one of the variants of the invention to the levels of any one of the corresponding original sequences from which it has been varied, or to compare the level of variants varied from one original sequence to levels of other variants varied from the same original which comparison may have some physiological meaning, for example for the indication of a physiological condition.

The invention also contemplates the use of the nucleic acid sequences as a diagnostic for diseases resulting from inherited defective sequences (of variants, TL or TH sequences), or diseases in which the ratio of the amount of the original sequence from which the variant was varied to the novel variants of the invention is altered. These sequences can be detected by comparing the sequences of the defective (i.e., mutant) coding region with that of a normal coding region. Association of the sequence coding for mutant product with abnormal valiant product activity may be verified. In addition, sequences encoding mutant products can be inserted into a suitable vector for expression in a functional assay system (e.g., colorimetric assay, complementation experiments in a variant protein deficient strain of HEK293 cells) as yet another means to verify or identify mutations. Once mutant genes have been identified, one can then screen populations of interest for carriers of the mutant gene.

Individuals carrying mutations in any one of the nucleic acid sequence of the present invention may be detected at the DNA level by a variety of techniques. Nucleic acids used for diagnosis may be obtained from a patient's cells, including but not limited to such as from blood, urine, saliva, placenta, tissue biopsy and autopsy material. Genomic DNA may be used directly for detection or may be amplified enzymatically by using PCR (Saiki, et al., Nature 324:163-166, (1986)) prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype.

Point mutations can be identified by hybridizing amplified DNA to radiolabeled RNA of the invention or alternatively, radiolabeled antisense DNA sequences of the invention. Sequence changes at specific locations may also be revealed by nuclease protection assays, such RNase and S1 protection or the chemical cleavage method (e.g. Cotton, et al., Proc. Natl. Acad. Sci. USA, 85:4397-4401, (1985)), or by differences in melting temperatures. “Molecular beacons” (Kosttikis L. G. et al., Science 279:1228-1229, (1998)), hair-pin-shaped, single-stranded synthetic oligo- nucleotides containing probe sequences which are complementary to the nucleic acid of the present invention, may also be used to detect point mutations or other sequence changes as well as monitor expression levels of the product. Such diagnostics would be particularly useful for prenatal testing.

Another method for detecting mutations uses two DNA probes which are designed to hybridize to adjacent regions of a target, with abutting bases, where the region of known or suspected mutation(s) is at or near the abutting bases. The two probes may be joined at the abutting bases, e.g., in the presence of a ligase enzyme, but only if both probes are correctly base paired in the region of probe junction. The presence or absence of mutations is then detectable by the presence or absence of ligated probe.

Also suitable for detecting mutations in the product coding sequence are oligonucleotide array methods based on sequencing by hybridization (SBH), as described, for example, in U.S. Pat. No. 5,547,83 9. In a typical method, the DNA target analyte is hybridized with an array of oligonucleotides formed on a microchip. The sequence of the target can then be “read” from the pattern of target binding to the array.

D. Gene Mapping Utilizing Nucleic Acid Sequences

The nucleic acid sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers (preferably 20-30 bp) from the cDNA. Computer analysis of the 3′ untranslated region is used to rapidly select primers that do not span more than one exon in the genomic DNA, which would complicate the amplification process. These primers are then used for PCR screening of somatic cell hybrids containing individual human chromosomes. Only those hybrids containing the human gene corresponding to the primer will yield an amplified fragment.

PCR mapping of somatic cell hybrids or using instead radiation hybrids are rapid procedures for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 50 or 60 bases. For a review of this technique, see Verma et al., Human Chromosomes: a Manual of Basic Techniques, (1988) Pergamon Press, New York.

Once a sequence has been mapped to a precise chromosomal location, the physical position of the sequence on the chromosome can be correlated with genetic map data. Such data are found, for example, in the OMIM database (Center for Medical Genetics, Johns Hopkins University, Baltimore, Md. and National Center for Biotechnology Information, National Library of Medicine, Bethesda, Md.). The OMIM gene map presents the cytogenetic map location of disease genes and other expressed genes. The OMIM database provides information on diseases associated with the chromosomal location. Such associations include the results of linkage analysis mapped to this interval, and the correlation of translocations and other chromosomal aberrations in this area.

E. Therapeutic Applications of Nucleic Acid Sequences

Nucleic acid sequences of the invention may also be used for therapeutic purposes. Turning first to the second embodiment of each aspect of the invention, inhibition of expression of any one of the products of the invention, expression of any one of the products may be modulated through antisense technology, which controls gene expression through hybridization of complementary nucleic acid sequences, i.e. antisense DNA or RNA, to the control, 5′ or regulatory regions of the gene encoding the product. For example, the 5′ coding portion of the nucleic acid sequence sequence which codes for the product of the present invention is used to design an antisense oligonucleotide of from about 10 to 40 base pairs in length. Oligonucleotides derived from the transcription start site, e.g. between positions −10 and +10 from the start site, are preferred. An antisense DNA oligonucleotide is designed to be complementary to a region of the nucleic acid sequence involved in transcription (Lee et al., Nucl. Acids, Res., 6:3073, (1979); Cooncy et al., Science 241:456, (1988); and Dervan et al., Science 251:1360, (1991)), thereby preventing transcription and the production of the variant products. An antisense RNA oligonucleotide hybridizes to the mRNA in vivo and blocks translation of the mRNA molecule into the products (Okano J Neurochem. 56:560, (1991)). The antisense constructs can be delivered to cells by procedures known in the art such that the antisense RNA or DNA may be expressed in vivo. The antisense may be antisense mRNA or DNA sequence capable of coding such antisense mRNA. The antisense mRNA or the DNA coding thereof can be complementary to the full sequence of nucleic acid sequences coding for any one of the products of the invention or to a fragment of such a sequence which is sufficient to inhibit production of the product.

Turning now to the expression of any one of the products of the invention may be increased by providing coding sequences for coding for said product under the control of suitable control elements ending its expression in the desired host.

The nucleic acid sequences of the invention may be employed in combination with a suitable pharmaceutical carrier. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The formulation should suit the mode of administration.

The products of the invention as well as any activators and deactivators compounds (see below) which are polypeptides, may also be employed in accordance with the present invention by expression of such polypeptides in vivo, which is often referred to as “gene therapy.” Cells from a patient may be engineered with a nucleic acid sequence (DNA or RNA) encoding a polypeptide ex vivo, with the engineered cells then being provided to a patient to be treated with the polypeptide. Such methods are well-known in the art. For example, cells may be engineered by procedures known in the art by use of a retroviral particle containing RNA encoding a polypeptide of the present invention.

Similarly, cells may be engineered in vivo for expression of a polypeptide in vivo by procedures known in the art. As known in the art, a producer cell for producing a retroviral particle containing RNA encoding the polypeptide of the present invention may be administered to a patient for engineering cells in vivo and expression of the polypeptide in vivo. These and other methods for administering a product of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle.

Retroviruses from which the retroviral plasmid vectors mentioned above may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, psi-2, psi-AM, PA12, T19-14X, VT-19-17-H2, psi-CRE, psi-CRIP, GP+E-86, GP+envAn12, and DAN cell lines as described in Miller (Human Gene Therapy, Vol. 1, pg. 5-14, (1990)). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

The genes introduced into cells may be placed under the control of inducible promoters, such as the radiation-inducible Egr-1 promoter, (Maceri, H. J., et al, Cancer Res., 56(19): 4311 (1996)), to stimulate production of products of the invention or antisense inhibition in response to radiation, eg., radiation therapy for treating tumors.

### EXAMPLE IV

### Products

The substantially purified product of the invention has been defined above as the product coded from the nucleic acid sequence of the invention. By its first embodiment the amino acid sequence is an amino acid sequence having at least 90% identity to any one of the sequences identified as SEQ ID NO: 42 to SEQ ID NO: 69 provided that the amino acid sequence is not identical to that of the original sequence from which it has been varied. The protein or polypeptide may be in mature and/or modified form, also as defined above. Also contemplated are protein fragments having at least 10 contiguous amino acid residues, preferably at least 10-20 residues, derived from the variant product, as well as homologues as explained above.

The sequence variations are preferably those that are considered conserved substitutions, as defined above. Thus, for example, a protein with a sequence having at least 90% sequence identity with any of the products identified as SEQ ID NO: 42 to SEQ ID NO: 69, preferably by utilizing conserved substitutions as defined above is also part of the invention, and provided that it is not identical to the original peptide from which it has been varied.

By a second aspect of the present invention, the amino acid sequence is an amino acid having at least 70% identity, preferably 80% identity, most preferably 90% identity to the sequences of SEQ ID NO: 70 (for TL) or 71 to 81 (for TH). The protein or polypeptide may be in a mature and/or modified form as defined above, and also contemplated are protein fragments having at least 10 amino acid residues, preferably 10-20 residues as well as homologs as defined above.

The sequence variations are preferably those that are considered conserved substitutions as defined above.

In a more specific embodiment, the protein has or contains any one of the sequence identified as SEQ ID NO: 42 to SEQ ID NO: 81. The variant product may be (i) one in which one or more of the amino acid residues in a sequence listed above are substituted with a conserved or non-conserved amino acid residue (preferably a conserved amino acid residue), or (ii) one in which one or more of the amino acid residues includes a substituent group, or (iii) one in which the variant product is fused with another compound, such as a compound to increase the half-life of the protein (for example, polyethylene glycol (PEG)), or a moiety which serves as targeting means to direct the protein to its target tissue or target cell population (such as an antibody), or (iv) one in which additional amino acids are fused to the variant product. Such fragments, variants and derivatives are deemed to be within the scope of those skilled in the art from the teachings herein.

A. Preparation of Products

Recombinant methods for producing and isolating the product of the invention, and fragments of the protein are described above.

In addition to recombinant production, fragments and portions of any one of the products may be produced by direct peptide synthesis using solid-phase techniques (cf. Stewart et al., (1969) Solid-Phase Peptide Synthesis, WH Freeman Co, San Francisco; Merrifield J., J. Am. Chem. Soc., 85:2149-2154, (1963)). In vitro peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. Fragments of the product may be chemically synthesized separately and combined using chemical methods to produce the full length molecule.

II. Therapeutic Uses and Compositions Utilizing the Product

The product of the invention is generally useful in treating diseases and disorders which are characterized by a lower than normal level of the product of the invention expression, and or diseases which can be cured or ameliorated by raising the level of the product of the invention, even if the level is normal.

Products or fragments may be administered by any of a number of routes and methods designed to provide a consistent and predictable concentration of compound at the target organ or tissue. The product-containing compositions may be administered alone or in combination with other agents, such as stabilizing compounds, and/or in combination with other pharmaceutical agents such as drugs or hormones.

Product-containing compositions may be administered by a number of routes including, but not limited to oral, intravenous, intramuscular transdermal, subcutaneous, topical, sublingual, or rectal means as well as by nasal application. Product-containing compositions may also be administered via liposomes. Such administration routes and appropriate formulations are generally known to those of skill in the art.

The product can be given via intravenous or intraperitoneal injection. Similarly, the product may be injected to other localized regions of the body. The product may also be administered via nasal insufflation. Enteral administration is also possible. For such administration, the product should be formulated into an appropriate capsule or elixir for oral administration, or into a suppository for rectal administration.

The foregoing exemplary administration modes will likely require that the product be formulated into an appropriate carrier; including ointments, gels, suppositories. Appropriate formulations are well known to persons skilled in the art.

Dosage of the product will vary, depending upon the potency and therapeutic index of the particular polypeptide selected.

A therapeutic composition for use in the treatment method can include the product in a sterile injectable solution, the polypeptide in an oral delivery vehicle, the product in an aerosol suitable for nasal administration, or the product in a nebulized form, all prepared according to well known methods. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof.

### EXAMPLE V

### Screening Methods for Activators and Deactivators (Inhibitors)

The present invention also includes an assay for identifying molecules, such as synthetic drugs, antibodies, peptides, or other molecules, which have a modulating effect on the activity of the product of the invention, e.g. activators or deactivators of the product of the present invention. Such an assay comprises the steps of providing a product encoded by the nucleic acid sequences of the present invention, contacting the product with one or more candidate molecules to determine the candidate molecules modulating effect on the activity of the product, and selecting from the molecules a candidate's molecule capable of modulating product physiological activity.

The variant product, the TL product or the TH product, its catalytic or immunogenic fragments or oligopeptides thereof, can be used for screening therapeutic compounds in any of a variety of drug screening techniques. The fragment employed in such a test may be tree in solution, affixed to a solid support, borne on a cell membrane or located intracellularly. The formation of binding complexes, between the product and the agent being tested, may be measured. Alternatively, the activator or deactivator may work by serving as agonist or antagonist, respectively, of the receptor of any one of the products, binding entity or target site, and their effect may be determined in connection with any of the above.

Another technique for drug screening which may be used provides for high throughput screening of compounds having suitable binding affinity to the variant product is described in detail by Geysen in PCT Application WO 84/03564, published on Sep. 13, 1984. In summary, large numbers of different small peptide test compounds are synthesized on a solid substrate, such as plastic pins or some other surface. The peptide test compounds are reacted with the full valiant product or with fragments of the product and washed. Bound product is then detected by methods well known in the art. Substantially purified product can also be coated directly onto plates for use in the aforementioned drug screening techniques. Alternatively, non-neutralizing antibodies can be used to capture the peptide and immobilize it on a solid support.

Antibodies to the product, as described in Example VI below, may also be used in screening assays according to methods well known in the art. For example, a “sandwich” assay may be performed, in which an anti-product antibody is affixed to a solid surface such as a microtiter plate and the product is added. Such an assay can be used to capture compounds which bind to the product. Alternatively, such an assay may be used to measure the ability of compounds to influence with the binding of the product of the invention to the receptor, and then select those compounds which effect the binding.

### EXAMPLE VI

### Anti-product-Antibodies

A. Synthesis

In still another aspect of the invention, the purified product of the invention is used to produce anti-product antibodies which have diagnostic and therapeutic uses related to the activity, distribution, and expression of the product.

Antibodies to the product may be generated by methods well known in the art. Such antibodies may include, but are not limited to, polyclonal, monoclonal, chimeric, humanized, single chain, Fab fragments and fragments produced by an Fab expression library. Antibodies, i.e., those which inhibit dimer formation, are especially preferred for therapeutic use.

A fragment of the product of the invention for antibody induction does not require biological activity but have to feature immunological activity; however, the protein fragment or oligopeptide must be antigenic. Peptides used to induce specific antibodies may have an amino acid sequence consisting of at least five amino acids, preferably at least 10 amino acids of the sequences specified in any one of SEQ ID NO: 42 to SEQ ID NO: 81. Preferably they should mimic a portion of the amino acid sequence of the natural protein and may contain the entire amino acid sequence of a small, naturally occurring molecule. Short stretches of product amino acids may be fused with those of another protein such as keyhole limpet hemocyanin and antibody produced against the chimeric molecule. Procedures well known in the art can be used for the production of antibodies to valiant product.

For the production of antibodies, various hosts including goats, rabbits, rats, mice, etc may be immunized by injection with the product or any portion, fragment or oligopeptide which retains immunogenic properties. Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Coiynebacterium parvum are potentially useful human adjuvants.

Monoclonal antibodies to the product may be prepared using any technique which provides for the production of antibody molecules by continuous cell lines in culture. These include but are not limited to the hybridoma technique originally described by Koehler and Milstein (Nature 256:495-497, (1975)), the human B-cell hybridoma technique (Kosbor et al., Immunol. Today 4:72, (1983); Cote et al., Proc. Natl. Acad. Sci. 80:2026-2030, (1983)) and the EBV-hybridoma technique (Cole, et al, Mol. Cell Biol. 62:109-120, (1984)).

Techniques developed for the production of “chimeric antibodies”, the splicing of mouse antibody genes to human antibody genes to obtain a molecule with appropriate antigen specificity and biological activity can also be used (Morrison et al., Proc. Natl. Acad. Sci. 81:6851-6855, (1984); Neuberger et al., Nature 312:604-608, (1984); Takeda et al., Nature 314:452-454, (1985)). Alternatively, techniques described for the production of single chain antibodies (U.S. Pat. No. 4,946,778) can be adapted to produce single-chain antibodies specific for the product of the invention

Antibodies may also be produced by inducing in vivo production in the lymphocyte population or by screening recombinant immunoglobulin libraries or panels of highly specific binding reagents as disclosed in Orlandi et al. (Proc. Natl. Acad. Sci. 86:3833-3837, 1989)), and Winter G and Milstein C., (Nature 349:293-299, (1991)).

Antibody fragments which contain specific binding sites for product protein may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W. D. et al., Science 256:1275-1281, (1989)).

B. Diagnostic Applications of Antibodies

A variety of protocols for competitive binding or immunoradiometric assays using either polyclonal or monoclonal antibodies with established specificities are well known in the art. Such immunoassays typically involve the formation of complexes between the product and its specific antibody and the measurement of complex formation. A two-site, monoclonal-based immunoassay utilizing monoclonal antibodies reactive to two noninterfering epitopes on a specific product is preferred, but a competitive binding assay may also be employed. These assays are described in Maddox D. E., et al., (J Exp. Med. 158:1211, (1983)).

Antibodies which specifically bind the product are useful for the diagnosis of conditions or diseases characterized by expression of any one of the products of the invention (where normally it is not expressed) by over or under expression of any one of the products of the invention as well as for detection of diseases in which the proportion between the amount of the variants of the invention and the original sequence from which it varied is altered. Alternatively, such antibodies may be used in assays to monitor patients being treated with any one of the products, its activators, or its deactivators. Diagnostic assays for products include methods utilizing the antibody and a label to detect the product in human body fluids or extracts of cells or tissues. The products and antibodies of the present invention may be used with or without modification. Frequently, the proteins and antibodies will be labeled by joining them, either covalently or noncovalently, with a reporter molecule. A wide variety of reporter molecules are known in the art.

A variety of protocols for measuring the product of the invention, using either polyclonal or monoclonal antibodies specific for the respective protein are known in the art. Examples include enzyme-linked immunosorbent assay (ELISA), radioimmunoassay (RIA), and fluorescent activated cell sorting (FACS). As noted above, a two-site, monoclonal-based immunoassay utilizing monoclonal antibodies reactive to two non-interfering epitopes on the product is preferred, but a competitive binding assay may be employed. These assays are described, among other places, in Maddox, et al. (supra). Such protocols provide a basis for diagnosing altered or abnormal levels of product expression. Normal or standard values for product expression are established by combining body fluids or cell extracts taken from normal subjects, preferably human, with antibody to the product under conditions suitable for complex formation which are well known in the art. The amount of standard complex formation may be quantified by various methods, preferably by photometric methods. Then, standard values obtained from normal samples may be compared with values obtained from samples from subjects potentially affected by disease. Deviation between standard and subject values establishes the presence of disease state.

The antibody assays are useful to determine the level of the product present in a body fluid sample, in order to determine whether it is being expressed at all, whether it is being overexpressed or underexpressed in the tissue, or as an indication of how levels of various products are responding to drug treatment.

By another aspect the invention concerns methods for determining the presence or level of various anti-product antibodies in a biological sample obtained from patients, such as blood or serum sample using as an antigen the valiant product. Determination of said antibodies may be indicative to a plurality of pathological conditions or diseases.

C. Therapeutic Uses of Antibodies

In addition to their diagnostic use the antibodies may have a therapeutical utility in blocking or decreasing the activity of any one of the products in pathological conditions where beneficial effect can be achieved by such a decrease.

The antibody employed is preferably a humanized monoclonal antibody, or a human Mab produced by known globulin-gene library methods. The antibody is administered typically as a sterile solution by IV injection, although other parenteral routes may be suitable. Typically, the antibody is administered in an amount between about 1-15 mg/kg body weight of the subject. Treatment is continued, e.g., with dosing every 1-7 days, until a therapeutic improvement is seen.

Although the invention has been described with reference to specific methods and embodiments, it is appreciated that various modifications and changes may be made without departing from the invention.

1. An isolated nucleic acid sequence selected from the group consisting of:
an isolated nucleic acid sequence, of an alternative splicing valiant, selected from the group consisting of:
(A)(i) the nucleic acid sequence depicted in any one of SEQ ID NO: 1 to SEQ ID NO: 28;
(A)(ii) nucleic acid sequences having at least 90% identity with the sequence of (A)(i) with the proviso that each sequence is different than the original nucleic acid sequence from which the sequences of (i) have been varied by alternative splicing; and
(A)(iii) fragments of (A)(i) or (A)(ii) of at least 20 b.p., provided that said fragment contains a sequence which is not present, as a continuous stretch of nucleotides, in the original nucleic acid sequence from which the sequences of (A)(i) have been varied by alternative splicing;
an isolated nucleic acid sequence selected from the group consisting of:
(B)(i) a nucleic acid sequence depicted in SEQ ID NO: 29 or 30;
(B)(ii) nucleic acid sequences having at least 70% identity with the sequence of (B)(i); and
(B)(iii) fragments of (B)(i) or (B)(ii) of at least 20 base pairs;
an isolated nucleic acid sequence selected from the group consisting of:
(C)(i) a nucleic acid sequence depicted in SEQ ID NO: 31 to 41.
(C)(ii) nucleic acid sequences having at least 70% identity with the sequence of (C)(i); and
(C)(iii) fragments of (C)(i) or (C)(ii) of at least 20 base pairs.

2. A nucleic acid sequence according to claim 1 (B)(ii) or claim 1 (C)(ii) wherein the nucleic acid sequences having at least 80% identity with the sequence of claim 1 (B)(i) or claim 1 (C)(i), respectively.

3. A nucleic acid sequence according to claim 2, wherein the nucleic acid sequences have at least 90% identity.

4. An isolated nucleic acid sequence complementary to the nucleic acid sequence of claim 1.

5. An amino acid sequence selected from the group consisting of:
(i) an amino acid sequence coded by the isolated nucleic acid sequence of alternative splice variants of claim 1 (A);
(ii) homologues of the amino acid sequences of 1(i) in which one or more amino acids has been added, deleted, replaced or chemically modified in the region or adjacent to the region where the amino acid sequences differs from the original amino acid sequence, coded by the original nucleic acid sequence from which the variant has been varied;
2(i) an amino acid sequence coded by the isolated nucleic acid sequence of claim 1(B);
2(ii) fragments of the amino acid sequence of claim 2(i) having at least 10 amino acids;
2(iii) analogs of the amino acid sequences of 2(i) or 2(ii) in which one or more amino acids has been added, deleted, replaced or chemically modified without substantially altering the biological activity of the parent amino acid sequence;
3(i) an amino acid sequence coded by the isolated nucleic acid sequence of claim 1(C);
3(ii) fragments of the amino acid sequence of claim 3(i) having at least 10 amino acids;
3(iii) analogs of the amino acid sequences of claim 3(i) or 3(ii) in which one or more amino acids has been added, deleted, replaced or chemically modified without substantially altering the biological activity of the parent amino acid sequence.

6. An amino acid sequence according to claim 5, as depicted in any one of SEQ ID NO: 42 to SEQ ID NO: 69.

7. An amino acid sequence according to claim 5 as depicted in SEQ ID NO: 70.

8. An amino acid sequence according to claim 5 as depicted in any one of SEQ ID NO: 71 to 81.

9. An isolated nucleic acid sequence coding for any one of the amino acid sequences of claim 6.

10. An isolated nucleic acid sequence coding for any one of the amino acid sequences of claim 7.

11. An isolated nucleic acid sequence coding for any one of the amino acid sequences of claim 8.

12. A purified antibody which binds specifically to any of the amino acid sequences of claim 6.

13. A purified antibody which binds specifically to any of the amino acid sequences of claim 7.

14. A purified antibody which binds specifically to any of the amino acid sequences of claim 8.

15. An expression vector comprising any one of the nucleic acid sequences of claim 1 and control elements for the expression of the nucleic acid sequence in a suitable host.

16. An expression vector comprising any one of the nucleic acid sequences of claim 4, and control elements for the expression of the nucleic acid sequences in a suitable host.

17. A host cell transfected by the expression vector of claim 15.

18. A host cell transfected by the expression vector of claim 16.

19. A pharmaceutical composition comprising a pharmaceutically acceptable carrier and as an active ingredient an agent selected from the group consisting of:
(i) the expression vector of claim 15; and
(ii) any one of the amino acid sequences of SEQ ID NO: 42 to 81, fragments of these amino acids having at least 10 amino acids and, analogs of said amino acid sequences.

20. A pharmaceutical composition according to claim 19, for treatment of diseases which can be ameliorated or cured by raising the level of any one of the amino acid sequences depicted in SEQ ID NO: 42 to SEQ ID NO: 81.

21. A pharmaceutical composition comprising a pharmaceutically acceptable carrier and as an active ingredient an agent selected from the group consisting of:
(i) any one of the nucleic acid sequences of claim 4;
(ii) the expression vector of claim 16; and
(iii) the purified antibody which specifically bind to any one off SEQ ID NO: 42 to 81.

22. A pharmaceutical composition according to claim 21, for treatment of diseases which can be ameliorated or cured by decreasing the level of any one of the amino acid sequences depicted in SEQ ID NO: 42 to SEQ ID NO: 81.

23. A method for detecting a nucleic acid sequence according to claim 1, in a biological sample, comprising the steps of:
(a) hybridizing to nucleic acid material of said biological sample any one of the nucleic acid sequences of claim 4; and
(b) detecting said hybridization complex;
wherein the presence of said hybridization complex correlates with the presence of said nucleic acid sequence in the said biological sample.

24. A method for determining the level of nucleic acid sequences of claim 1 in a biological sample comprising the steps of:
(a) hybridizing to nucleic acid material of said biological sample any one of the nucleic acid sequences of claim 4; and
(b) determining the amount of hybridization complexes and normalizing said amount to provide the level of the nucleic acid sequences in the sample.

25. A method, for determining the level of any one of the nucleic acid sequences of claim 1 in a biological sample comprising the steps of:
(i) contacting the sample with probes for amplification of any one of SEQ ID NO: 1 to SEQ ID NO: 41;
(ii) proving reagents for amplification;
(iii) detecting the presence of amplified products,
presence of said products indicating the presence of the nucleic acid in the sample.

26. A method for determining the ratio between the level of variant of the nucleic acid sequence in a first biological sample and the level of the original sequence from which the variant has been varied by alternative splicing in a second biological sample comprising:
(a) determining the level of the variant nucleic acid sequence according to claim 1(A) in the fist biological sample according to the method of claim 24;
(b) determining the level of the original sequence in the second biological sample; and
(c) comprising the levels obtained in (a) and (b) to give said ratio.

27. A method according to claim 26, wherein said first and said second biological samples are the same sample.

28. A method according to any of claim 23, wherein the nucleic acid material of said biological sample are mRNA transcripts.

29. A method according to claim 28, where the nucleic acid sequence is present in a nucleic acid chip.

30. A method for detecting any one of the amino acid sequences of depicted in SEQ ID NO: 42 to 69 in a biological sample, comprising the steps of:
(a) contacting with said biological sample the antibody of claim 12, thereby forming an antibody-antigen complex; and
(b) detecting said antibody-antigen complex
wherein the presence of said antibody-antigen complex correlates with the presence of the desired amino acid in said biological sample.

31. A method for detecting the level of the amino acid sequence of SEQ ID NO: 70 in a biological sample, comprising the steps of:
(a) contacting with said biological sample the antibody of claim 13, thereby forming an antibody-antigen complex; and
(b) detecting the amount of said antibody-antigen complex and normalizing said amount to provide the level of said amino acid sequence in the sample.

32. A method for detecting the amino acid sequences of SEQ ID NO: 71 to 81 in a biological sample comprising the step of:
(a) contacting the biological sample with the antibody of claim 14, thereby forming an antibody-antigen complex; and
(b) detecting the amount of said antibody-antigen complex and normalizing said amount to provide the level of said amino acid sequence in the sample.

33. A method for determining the ratio between the level of any one of the amino acid sequences depicted in SEQ ID NO: 42 to SEQ ID NO: 69 present in a first biological sample and the level of the original amino acid sequences from which they were varied by alternative splicing, present in a second biological sample, the method comprising:
(a) determining the level of the amino acid sequences in a first sample by the method of claim 30;
(b) determining the level of the original amino acid sequence in the second sample; and
(c) comparing the level obtained in (a) and (b) to give said ratio.

34. A method according to claim 33, wherein said first and said second biological samples are the same sample.

35. A method for detecting any one of the antibodies of claim 12 in a biological sample comprising the steps of:
(a) contacting said biological sample with any one of the amino acid sequences depicted in SEQ ID NO: 42 to 69 thereby forming an antibody-antigen complex; and
(b) detecting said antibody-antigen complex
wherein the presence of said antibody-antigen complex correlates with the presence of the antibody in said biological sample.
